(19) United States
(12) Patent Application Publication
(10) Pub. No.: US 2004/0203022 A1
(43) Pub. Date: Oct. 14, 2004

(54) Proteins and genes for diagnosis and treatment of ErbB2-related cancer

(76) Inventors: Herath Mudiyanselage Athula Chandrasiri Herath, Abingdon (GB); Martin John Page, Abingdon (GB)

Correspondence Address: KLAUBER & JACKSON, 411 HACKENSACK AVENUE, HACKENSACK, NJ, 07601

(21) Appl. No.: 10/700,330

(22) Filed: Nov. 3, 2003

Related U.S. Application Data
(63) Continuation of application No. PCT/GB02/02047, filed on May 2, 2002.

(30) Foreign Application Priority Data
May 3, 2001 (GB) 0110886.9
Nov. 23, 2001 (GB) 0128183.1

(51) Int. Cl. C12Q 1/68; G01N 33/574

(52) U.S. Cl. 435/6; 435/7.23

(57) ABSTRACT

The present invention relates to the identification of polypeptides, proteins and protein isoforms that are associated with ErbB-2 related cancer and its onset and development, of genes encoding the same, and to their use for e.g., clinical screening, diagnosis, prognosis, therapy and prophylaxis, as well as for drug screening and drug development.

### RELATED APPLICATIONS

The present application is a Continuation of co-pending PCT Application No. PCT/GB02/02047 filed May 2, 2002, which in turn, claims priority from Great Britain Application Serial No. 0110886.9, filed on May 3, 2001 and Great Britain Application No. 0128183.1, filed on Nov. 23, 2001. Applicants claim the benefits of 35 U.S.C. §120 as to the PCT application and priority under 35 U.S.C. § 119 as to the said Great Britain applications, and the entire disclosures of all applications are incorporated herein in their entireties.

### INTRODUCTION

The present invention relates to the identification of polypeptides, proteins and protein isoforms that are associated with ErbB-2 related cancer and its onset and development, of genes encoding the same, and to their use for e.g., clinical screening, diagnosis, prognosis, therapy and prophylaxis, as well as for drug screening and drug development.

### BACKGROUND OF THE INVENTION

Breast cancer is the most frequently diagnosed non-skin cancer among women in the United States. It is second only to lung cancer in cancer-related deaths. Approximately 180,000 new cases of breast cancer were diagnosed in 1997 in the US, and about 44,000 women were expected to die from the disease. A report from the National Cancer Institute (NCI) estimates that about 1 in 8 women in the United States (approximately 12.8 percent) will develop breast cancer during her lifetime. This estimate is based on data from NCI's Surveillance, Epidemiology, and End Results Program (SEER) publication SEER Cancer Statistics Review 1973-1997 and is based on cancer rates from 1995 through 1997. (National Cancer Institute, www.cancernet.nci.nih.gov, USA, 1999). In the UK, breast cancer is by far the commonest cancer for women, with 34,600 new cases in 1998 (Cancer Research Campaign, www.crc.org.uk, UK, 2000). Ninety-nine percent of breast cancers occur in women. The annual cost of breast cancer treatment in the United States is approximately $10 billion (Fuqua, et. al. 1998, American Association for Cancer Research, www.aacr.org, USA). Breast cancer incidence has been rising over the past five decades, but recently it has plateaued. This may reflect a period of earlier detection of breast cancers by mammography. A number of established factors can increase a woman's risk of having the disease. These include older age, history of prior breast cancer, significant radiation exposure, strong family history of breast cancer, upper socioeconomic class, nulliparity, early menarche, late menopause, or age at first pregnancy greater than 30 years. Prolonged use of oral contraceptives earlier in life appears to increase risk slightly. Prolonged postmenopausal oestrogen replacement increases the risk 20 to 40%. It has been speculated that a decrease in the age at menarche, changing birth patterns, or a rise in the use of exogenous estrogens has contributed to the increase in breast cancer incidence (Fuqua, et. al. 1998, American Association for Cancer Research, www.aacr.org, USA).

### Involvement of ErbB2 in Breast Cancer

The ErbB2 oncogene encodes a 185 kDa transmembrane glycoprotein that belongs to the epidermal growth factor family of Type I receptor tyrosine kinases. It is known that ErbB2/HER2 is the preferred heterodimerization partner for all the family of ErbB receptors (EGFR, ErbB3, ErbB4) and that it can mediate signal transduction for all these receptors when they bind their cognate ligands (for example EGF, amphiregulin, crypto, heregulins). ErbB2 therefore potentially plays a key role in the variety of signals normally transmitted by this receptor family, including growth, differentiation and resistance to apoptosis. The role of ErbB2 in human malignancy was identified within a year of the isolation of the ErbB2 human gene in 1986. It is amplified at the gene level and/or over-expressed at the protein level in around 30% of human breast carcinomas. This observation has been amply confirmed in some 50 studies and 15,000 patients using principally immunohistochemistry (IHC), but more recently fluorescence in situ hybridization (FISH) and ELISA. Over-expression of ErbB2 has also been found, albeit less frequently, in other epithelial malignancies such as those of the ovary, stomach and lung. (Ross, J. S. & Fletcher, J. A. The HER2/neu oncogene in breast cancer: prognostic factor, predictive factor, and target for therapy. The Oncologist 1998; 3: 237-252)

Over-expression of ErbB2 has been shown to correlate with poor clinical prognosis in breast cancer, and there is experimental evidence that, where it is over-expressed, this contributes to the malignant phenotype of the tumour. Over-expression of ErbB2 leads to constitutive activation of the tyrosine kinase receptor, and to stimulation of downstream signalling pathways that terminate in increased mitogenesis and resistance to apoptosis. It has therefore long been postulated that ErbB2 would be a worthwhile target in the management of those breast cancers in which it is over-expressed. This expectation has now been realised by the trastuzumab/Herceptin™ antibody, setting a precedent for further exploitation of ErbB2 related targets. (Ross, J. S. & Fletcher, J. A. The HER2/neu oncogene in breast cancer: prognostic factor, predictive factor, and target for therapy. The Oncologist 1998; 3: 237-252)

New Therapeutic Opportunities Offered by Understanding ErbB2 Transformation

The degree of cellular ErbB2 overexpression is directly linked to the severity of ErbB2-related cancer. Now that the potential of ErbB2 as a therapeutic target in breast cancer, and other cancers in which it is overexpressed is clearly established, the possibility exists of exploiting and extending this target in various ways. Although most therapeutic effort has concentrated on attacking ErbB2 itself, new opportunities would be offered by an understanding in detail of how overexpression of ErbB2 affects cells, and thus the precise mechanism by which it transforms normal cells to cancerous cells. A few proteins are known which are in direct signaling pathways from the ErbB2 protein, but little is known about the global changes in the protein repertoire of cells consequent on transformation by ErbB2 overexpression.

Therefore, a need exists to identify sensitive and specific biomarkers for the diagnosis of ErbB2-related cancer in living subjects. Additionally, there is a clear need for new therapeutic agents for ErbB2-related cancer that work quickly, potently, specifically and with fewer side effects.

An example of how epithelial breast cancer markers have been identified previously using 2D PAGE electrophoresis is given in Page et al., (1999) Proc Natl Acad Sci USA, 96; 12589-12594.

### SUMMARY OF THE INVENTION

The present invention provides methods and compositions for clinical screening, diagnosis and treatment of ErbB2 related cancer, for monitoring the effectiveness of ErbB2 related cancer treatment, for selecting participants in clinical trials, for identifying patients most likely to respond to a particular therapeutic treatment and for screening and development of drugs for treatment of ErbB2 related cancer.

The invention provides methods for diagnosis of ErbB2 related cancer that comprise analyzing a sample of tissue or body fluid by two-dimensional electrophoresis to detect the presence or level of at least one ErbB2 Overexpression-Associated Feature (EOF) disclosed herein or any combination thereof. These methods are also suitable for clinical screening, prognosis, monitoring the results of therapy, identifying patients most likely to respond to a particular therapeutic treatment, for drug screening and development, and identification of new targets for drug treatment.

The invention also provides methods for diagnosis of ErbB2 related cancer that comprise detecting in a sample of tissue or body fluid the presence or level of at least one ErbB2 Overexpression-Associated Protein Isoform (EOPI) disclosed herein or any combination thereof. These methods are also suitable for clinical screening, prognosis, monitoring the results of therapy, identifying patients most likely to respond to a particular therapeutic treatment, drug screening and development, and identification of new targets for drug treatment.

The invention also provides antibodies, e.g. monoclonal and polyclonal, chimeric and humanised antibodies capable of immunospecific binding to an EOPI.

The invention also provides a preparation comprising an isolated EOPI, i.e., an EOPI substantially free from polypeptides, proteins or protein isoforms having a significantly different isoelectric point or a significantly different apparent molecular weight from the EOPI.

The invention also provides kits that may be used in the above recited methods and that may comprise single or multiple preparations, or antibodies, together with other reagents, labels, substrates, if needed, and directions for use. The kits may be used for diagnosis of disease, or may be assays for the identification of new diagnostic and/or therapeutic agents.

The invention also provides methods of treating ErbB2 related cancer, comprising administering to a subject a therapeutically effective amount of an agent that modulates (e.g., upregulates or downregulates) the expression or activity (e.g. enzymatic or binding activity), or both, of an EOPI in subjects having ErbB2 related cancer, in order to prevent or delay the onset or development of ErbB2 related cancer, to prevent or delay the progression of ErbB2 related cancer, or to ameliorate the symptoms of ErbB2 related cancer.

The invention also provides methods of screening for agents that modulate (e.g., upregulate or downregulate) a characteristic of, e.g., the expression or the enzymatic or binding activity, of a EOF, EOPI, a EOPI analog, or a EOPI-related polypeptide.

### BRIEF DESCRIPTION OF THE FIGURES

FIG. 1 is a flow chart depicting the characterization of an EOF and relationship of a EOF and EOPI. An EOF may be further characterized as or by an EOPI having a particular peptide sequence associated with its pI and MW. As depicted herein, an EOF may comprise one or more EOPIs, which have indistinguishable pIs and MWs using the Preferred Technology, but which have distinct peptide sequences. The peptide sequence(s) of the EOPI can be utilized to search database(s) for previously identified proteins comprising such peptide sequence(s). It can be ascertained whether a commercially available antibody exists that may recognize the previously identified protein and/or a member of its protein family.

FIG. 2 is an image obtained from 2-dimensional electrophoresis of cell lysate obtained from an ErbB2 overexpressing cell line, which has been annotated to identify thirteen landmark features, designated BT1 to BT13.

FIG. 3 is a Venn diagram depicting the number of EOFs identified amongst the moderately (Venn position A) and highly (Venn position C) overexpressing ErbB2 cell lines compared to the control (no ErbB2 over-expression) cell line. An overlap of EOFs identified in both moderately and highly overexpressing ErbB2 cell lines (Venn position B) was also identified.

### DETAILED DESCRIPTION OF THE INVENTION

The invention described in detail below provides methods and compositions for clinical screening and diagnosis of ErbB2 related cancer in a mammalian subject for identifying patients most likely to respond to a particular therapeutic treatment, for monitoring the results of ErbB2 related cancer therapy, for drug screening and drug development. The invention also encompasses the administration of therapeutic compositions to a mammalian subject to treat or prevent ErbB2 related cancer. The mammalian subject may be a non-human mammal, but is preferably human, more preferably a human adult, e.g. a human subject at least 21 years old.

For clarity of disclosure, and not by way of limitation, the invention will be described with respect to the analysis of cell lysates of breast cell lines. However, as one skilled in the art will appreciate, the assays and techniques described below can be applied to other types of samples, including a body fluid (for example but without limitation: blood, serum, plasma, saliva or urine), a tissue sample from a subject at risk of having or developing ErbB2 related cancer (e.g. a biopsy such as a breast biopsy) or homogenate thereof. The methods and compositions of the present invention are useful for screening and diagnosis of a living subject, but may also be used for postmortem diagnosis in a subject, for example, to identify family members of the subject who are at risk of developing the same disease.

The following definitions are provided to assist in the review of the instant disclosure.

Definitions

“ErbB2” refers to the oncogene, which is also known as “HER2” or “Neu”.

“ErbB2-related cancer” refers to a cancer, which displays overexpression of the ErbB2 oncogene, and can be for example breast, ovary, stomach or lung cancer.

“Feature” refers to a spot identified in a 2D gel, and the term “ErbB2 related cancer—Associated Feature” (EOF) refers to a feature that is differentially present in a first sample or sample set from a subject having ErbB2 related cancer compared with a second sample or sample set from a subject free from ErbB2 related cancer. A feature or spot identified in a 2D gel is characterized by its isoelectric point (pI) and apparent molecular weight (MW) as determined by 2D gel electrophoresis, particularly utilizing the Preferred Technology described herein. As used herein, a feature is “differentially present” in a first sample or sample set with respect to a second sample or sample set when a method for detecting the said feature (e.g., 2D electrophoresis) gives a different signal when applied to the first and second samples or sample sets. An EOF, (or a Protein Isoform, i.e. EOPI, as defined infra) is “increased” in the first sample or sample set with respect to the second sample or sample set if the method of detection indicates that the EOF, or EOPI is more abundant in the first sample or sample set than in the second sample or sample set, or if the EOF, or EOPI is detectable in the first sample or sample set and substantially undetectable in the second sample or sample set. Conversely, an EOF, or EOPI is “decreased” in the first sample or sample set with respect to the second sample or sample set if the method of detection indicates that the EOF, or EOPI is less abundant in the first sample or sample set than in the second sample or sample set or if the EOF, or EOPI is undetectable in the first sample or sample set and detectable in the second sample or sample set.

Particularly, the relative abundance of a feature in the two samples or sample sets is determined in reference to its normalized signal, in two steps. First, the signal obtained upon detecting the feature in a first sample or sample set is normalized by reference to a suitable background parameter, e.g., (a) to the total protein in the sample being analyzed (e.g., total protein loaded onto a gel); (b) to an Expression Reference Feature (ERF) i.e., a feature whose abundance is substantially invariant, within the limits of variability of the Preferred Technology, in the population of subjects being examined, e.g. the ERFs disclosed in Table III, or (c) more preferably to the total signal detected as the sum of each of all proteins in the sample.

Secondly, the normalized signal for the feature in the first sample or sample set is compared with the normalized signal for the same feature in the second sample or sample set in order to identify features that are “differentially present” in the first sample or sample set with respect to the second sample or sample set.

“Fold change” includes “fold increase” and “fold decrease” and refers to the relative increase or decrease in abundance of an EOF or the relative increase or decrease in expression or activity of a polypeptide (e.g. an EOPI, as defined infra.) in a first sample or sample set compared to a second sample or sample set. An EOF or polypeptide fold change may be measured by any technique known to those of skill in the art, albeit the observed increase or decrease will vary depending upon the technique used. Preferably, fold change is determined herein as described in the Examples infra.

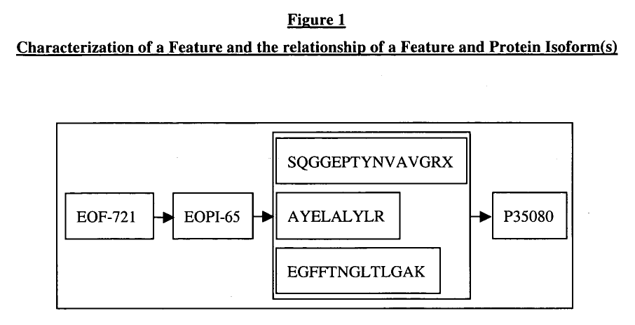

“ErbB2 related cancer-Associated Protein Isoform” (EOPI) refers to a polypeptide that is differentially present in a first sample or sample set from a subject having ErbB2 related cancer compared with a second sample or sample set from a subject free from ErbB2 related cancer. As used herein, an EOPI is “differentially present” in a first sample or sample set with respect to a second sample or sample set when a method for detecting the said feature, (e.g., 2D electrophoresis or immunoassay) gives a different signal when applied to the first and second samples or sample sets (as described above in relation to EOFs). An EOPI is characterised by one or more peptide sequences of which it is comprised, and further by a pI and MW, preferably determined by 2D electrophoresis, particularly utilising the Preferred Technology as described herein. Typically, EOPIs are identified or characterised by the amino acid sequencing of EOFs (FIG. 1).

An EOPI is characterized as, or by, a particular peptide sequence associated with its pI and MW. As depicted herein, an EOF may comprise one or more EOPI(s), which have indistinguishable pIs and MWs using the Preferred Technology, but which have distinct peptide sequences. The peptide sequence(s) of the EOPI can be utilized to search database(s) for previously identified proteins comprising such peptide sequence(s). In some instances, it can be ascertained whether a commercially available antibody exists which may recognize the previously identified protein and/or a variant thereof. Prefereably the EOPI corresponds to the previously identified protein, or be a variant of the previously identified protein.

“Variant” as used herein refers to a polypeptide which is a member of a family of polypeptides that are encoded by a single gene or from a gene sequence within a family of related genes and which differ in their pI or MW, or both. Such variants can differ in their amino acid composition (e.g. as a result of alternative mRNA or premRNA processing, e.g. alternative splicing or limited proteolysis) and in addition, or in the alternative, may arise from differential post-translational modification (e.g., glycosylation, acylation, phosphorylation).

“Modulate” in reference to expression or activity of an EOF, EOPI or EOPI-related polypeptide refers to any change, e.g., upregulation or downregulation, increase or decrease, of the expression or activity of the EOF, EOPI or EOPI-related polypeptide. Those skilled in the art, based on the present disclosure, will understand that such modulation can be determined by assays known to those of skill in the art.

“EOPI analog” refers to a polypeptide that possesses similar or identical function(s) as an EOPI but need not necessarily comprise an amino acid sequence that is similar or identical to the amino acid sequence of the EOPI, or possess a structure that is similar or identical to that of the EOPI. As used herein, an amino acid sequence of a polypeptide is “similar” to that of a EOPI if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the EOPI; (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the EOPI; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the EOPI. As used herein, a polypeptide with “similar structure” to that of a EOPI refers to a polypeptide that has a similar secondary, tertiary or quarternary structure as that of the EOPI. The structure of a polypeptide can determined by methods known to those skilled in the art, including but not limited to, X-ray crystallography, nuclear magnetic resonance, and crystallographic electron microscopy.

“EOPI fusion protein” refers to a polypeptide that comprises (i) an amino acid sequence of an EOPI, EOPI fragment, EOPI-related polypeptide or a fragment of an EOPI-related polypeptide and (ii) an amino acid sequence of a heterologous polypeptide (i.e., a non-EOPI, non-EOPI fragment or non-EOPI-related polypeptide).

“EOPI homolog” refers to a polypeptide that comprises an amino acid sequence similar to that of an EOPI but does not necessarily possess a similar or identical function as the EOPI.

“EOPI ortholog” refers to a non-human polypeptide that (i) comprises an amino acid sequence similar to that of an EOPI and (ii) possesses a similar or identical function to that of the EOPI.

“EOPI-related polypeptide” refers to an EOPI homolog, an EOPI analog, a variant of an EOPI, an EOPI ortholog, or any combination thereof.

“Chimeric Antibody” refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397, which are incorporated herein by reference in their entirety). For example, a portion of the antibody may be fused with the constant domain of immunoglobulins (IgA, IgE, IgG, IgM), or portions thereof (CH1, CH2, CH3, or any combination thereof and portions thereof) resulting in chimeric antibodies.

“Humanised Antibody” refers to a molecule from non-human species having one or more complementary determining regions (CDRs) from the non-human species and a framework region from a human immunoglobulin molecule.

“Derivative” refers to a polypeptide that comprises an amino acid sequence of a second polypeptide that has been altered by the introduction of at least one amino acid residue substitution, deletion or addition. The derivative polypeptide possesses a similar or identical function as the second polypeptide.

“Fragment” refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a second polypeptide. Preferably the fragment of a EOPI possesses the functional activity of the EOPI.

“Percent identity” refers to the number of identical amino acid residues (or nucleic acid bases) as a percentage of the length of sequence of the polypeptide (or its encoding nucleic acid sequence).

The “percent identity” of two amino acid sequences or of two nucleic acid sequences can be or is generally determined by aligning the sequences for optimal comparison purposes (e.g., gaps can be introduced in either sequences for best alignment with the other sequence) and comparing the amino acid residues or nucleotides at corresponding positions. The “best alignment” is an alignment of two sequences that results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to a protein molecule of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-BLAST programs, the default parameters of the respective programs (e.g., XBLAST and NBLAST) can be used.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

“Diagnosis” refers to diagnosis, prognosis, monitoring, characterizing, selecting patients, including participants in clinical trials, and identifying patients at risk for or having a particular disorder or those most likely to respond to a particular therapeutic treatment, or for assessing or monitoring a patient's response to a particular therapeutic treatment.

“Treatment” refers to therapy, prevention and prophylaxis and particularly refers to the administration of medicine or the performance of medical procedures with respect to a patient, for either prophylaxis (prevention) or to cure the infirmity or malady in the instance where the patient is afflicted.

“Agent” refers to all materials that may be used to prepare pharmaceutical and diagnostic compositions, or that may be compounds, agonists, antagonists, nucleic acids, polypeptides, fragments, isoforms, variants, or other materials that may be used independently for such purposes, all in accordance with the present invention.

“Highly stringent conditions” refers to hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & Sons, Inc., New York, at p. 2.10.3; incorporated herein by reference in its entirety.)

For some applications, less stringent conditions for duplex formation are required. As used herein “moderately stringent conditions” refers to washing in 0.2×SSC/0.1% SDS at 42° C. (Ausubel et al., 1989, supra).

“Serum” refers to the supernatant fluid produced by clotting and centrifugal sedimentation of a blood sample.

“Plasma” refers to the supernatant fluid produced by inhibition of clotting (for example, by citrate or EDTA) and centrifugal sedimentation of a blood sample.

“Blood” as used herein includes serum and plasma.

“Two-dimensional electrophoresis” (2D-electrophoresis) means a technique comprising denaturing electrophoresis, followed by isoelectric focusing; this generates a two-dimensional gel (2D-gel) containing a plurality of separated proteins.

The “Preferred Technology”

Preferably, the step of denaturing electrophoresis uses polyacrylamide electrophoresis in the presence of sodium dodecyl sulfate (SDS-PAGE). Especially preferred are the highly accurate and automatable methods and apparatus (“the Preferred Technology”) described in WO 98/23950 and in U.S. Pat. Nos 6,064,654, and 6,278,794, each of which is incorporated herein by reference in its entirety with particular reference to the protocol at pages 23-35 of WO 98/23950. Briefly, the Preferred Technology provides efficient, computer-assisted methods and apparatus for identifying, selecting and characterising biomolecules (e.g. proteins, including glycoproteins) in a biological sample. A two-dimensional array is generated by separating biomolecules on a two-dimensional gel according to their electrophoretic mobility and isoelectric point. A computer-generated digital profile of the array is generated, representing the identity, apparent molecular weight, isoelectric point, and relative abundance of a plurality of biomolecules detected in the two-dimensional array, thereby permitting computer-mediated comparison of profiles from multiple biological samples, as well as computer aided excision of separated proteins of interest.

A preferred scanner for detecting fluorescently labelled proteins is described in WO 96/36882 and in the Ph.D. thesis of David A. Basiji, entitled “Development of a High-throughput Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686, the contents of each of which are incorporated herein by reference. These documents describe an image scanner designed specifically for automated, integrated operation at high speeds. The scanner can image gels that have been stained with fluorescent dyes or silver stains, as well as storage phosphor screens. The Basiji thesis provides a phase-sensitive detection system for discriminating modulated fluorescence from baseline noise due to laser scatter or homogeneous fluorescence, but the scanner can also be operated in a non-phase-sensitive mode. This phase-sensitive detection capability would increase the sensitivity of the instrument by an order of magnitude or more compared to conventional fluorescence imaging systems. The increased sensitivity would reduce the sample-preparation load on the upstream instruments while the enhanced image quality simplifies image analysis downstream in the process.

A more highly preferred scanner is a modified version of the scanner described above. In the preferred scanner, the gel is transported through the scanner on a precision lead-screw drive system. This is preferable to laying the glass plate on the belt-driven system that is described in the Basiji thesis, as it provides a reproducible means of accurately transporting the gel past the imaging optics.

In the preferred scanner, the gel is secured against three alignment stops that rigidly hold the glass plate in a known position. By doing this in conjunction with the above precision transport system, the absolute position of the gel can be predicted and recorded. This ensures that co-ordinates of each feature on the gel can be determined more accurately and communicated, if desired, to a cutting robot for excision of the feature. In the preferred scanner, the carrier that holds the gel has four integral fluorescent markers for use to correct the image geometry. These markers are a quality control feature that confirms that the scanning has been performed correctly.

In comparison to the scanner described in the Basiji thesis, the optical components of the preferred scanner have been inverted. In the preferred scanner, the laser, mirror, waveguide and other optical components are above the glass plate being scanned. The scanner described in the Basiji thesis has these components underneath. In the preferred scanner, the glass plate is mounted onto the scanner gel side down, so that the optical path remains through the glass plate. By doing this, any particles of gel that may break away from the glass plate will fall onto the base of the instrument rather than into the optics. This does not affect the functionality of the system, but increases its reliability.

Still more preferred is a modified version of the preferred scanner, in which the signal output is digitised to the full 16-bit data without any peak saturation or without square root encoding of the signal. A compensation algorithm has also been applied to correct for any variation in detection sensitivity along the path of the scanning beam. This variation is due to anomalies in the optics and differences in collection efficiency across the waveguide. A calibration is performed using a perspex plate with an even fluorescence throughout. The data received from a scan of this plate are used to determine the multiplication factors needed to increase the signal from each pixel level to a target level. These factors are then used in subsequent scans of gels to remove any internal optical variations.

### Overexpression-Associated Features (EOFs)

In one aspect of the invention, two-dimensional electrophoresis is used to analyze tissue or body fluid from a subject, preferably a living subject, in order to detect or quantify the expression of one or more ErbB2 related cancer-Associated Features (EOFs) for screening, prevention or diagnosis of ErbB2 related cancer, to determine the prognosis of a subject having ErbB2 related cancer, to monitor progression of ErbB2 related cancer, to monitor the effectiveness of ErbB2 related cancer therapy, for identifying patients most likely to respond to a particular therapeutic treatment, or for drug development.

By way of example and not of limitation, using the Preferred Technology, a number of samples from subjects having ErbB2 related cancer and samples from subjects free from ErbB2 related cancer are separated by two-dimensional electrophoresis, and the fluorescent digital images of the resulting gels are matched to a chosen representative primary master gel image. This process allows any gel feature, characterised by its pI and MW, to be identified and examined on any gel of the study. In particular, the amount of protein present in a given feature can be measured in each gel; this feature abundance can be averaged amongst gels from similar samples (e.g. gels from samples from subjects having ErbB2 related cancer). Finally, statistical analyses can be conducted on the thus created sample sets, in order to compare 2 or more sample sets to each other.

The EOFs disclosed herein have been identified by comparing two cell lines, one, which overexpresses ErbB2 moderately and another, which overexpresses ErbB2 highly, with a control (no ErbB2 overexpression) cell line. The EOFs have been identified through the methods and apparatus of the Preferred Technology and can be described by apparent molecular weight (MW) and isoelectric point (pI) as provided in Table I. Preferred EOFs of the invention are EOF-86, EOF-106, EOF-163, EOF-183, EOF-201, EOF-396, EOF-483, EOF-630, EOF-634, EOF-683 and EOF-693.

TABLE I
EOFs identified in cells overexpressing ErbB2

| Feature | | |
|---|---|---|
| (EOF) | pI | MW (Da) |
| EOF-86 | 6.16 | 47,684 |
| EOF-106 | 5.08 | 40,638 |
| EOF-163 | 4.74 | 14,222 |
| EOF-183 | 5.04 | 105,577 |
| EOF-201 | 6.37 | 43,168 |
| EOF-396 | 5.78 | 104,459 |
| EOF-483 | 5.39 | 127,995 |
| EOF-630 | 4.98 | 21,851 |
| EOF-634 | 5.05 | 16,573 |
| EOF-683 | 5.45 | 13,968 |
| EOF-693 | 4.99 | 10,445 |
| EOF-34 | 6.29 | 113,362 |
| EOF-35 | 6.51 | 110,562 |
| EOF-44 | 6.44 | 85,990 |
| EOF-48 | 4.93 | 80,273 |
| EOF-51 | 6.61 | 74,208 |
| EOF-57 | 5.16 | 68,895 |
| EOF-61 | 7.73 | 61,037 |
| EOF-63 | 5.02 | 58,032 |
| EOF-66 | 5.47 | 56,144 |
| EOF-67 | 6.92 | 56,452 |
| EOF-70 | 4.80 | 54,791 |
| EOF-72 | 5.58 | 52,905 |
| EOF-73 | 4.76 | 53,395 |
| EOF-74 | 5.27 | 52,894 |
| EOF-75 | 5.16 | 51,876 |
| EOF-76 | 4.83 | 52,196 |
| EOF-76 | 4.83 | 52,196 |
| EOF-80 | 4.67 | 51,143 |
| EOF-81 | 5.17 | 49,168 |
| EOF-81 | 5.17 | 49,168 |
| EOF-83 | 5.06 | 49,351 |
| EOF-84 | 4.77 | 49,233 |
| EOF-87 | 4.61 | 47,205 |
| EOF-90 | 5.39 | 44,908 |
| EOF-94 | 6.13 | 44,019 |
| EOF-99 | 5.40 | 42,809 |
| EOF-102 | 5.39 | 42,227 |
| EOF-104 | 6.72 | 41,786 |
| EOF-107 | 5.74 | 40,619 |
| EOF-110 | 6.16 | 39,933 |
| EOF-111 | 4.77 | 40,050 |
| EOF-120 | 5.77 | 36,187 |
| EOF-129 | 4.61 | 31,848 |
| EOF-133 | 5.16 | 31,009 |
| EOF-135 | 5.60 | 30,301 |
| EOF-136 | 5.14 | 29,942 |
| EOF-137 | 5.39 | 29,939 |
| EOF-138 | 6.10 | 29,850 |
| EOF-139 | 5.21 | 29,877 |
| EOF-145 | 5.04 | 28,469 |
| EOF-149 | 5.54 | 23,266 |
| EOF-153 | 5.45 | 21,277 |
| EOF-156 | 5.06 | 19,901 |
| EOF-166 | 4.96 | 12,052 |
| EOF-167 | 5.26 | 12,062 |
| EOF-182 | 5.10 | 106,575 |
| EOF-188 | 6.29 | 69,345 |
| EOF-196 | 5.55 | 50,803 |
| EOF-197 | 5.59 | 50,859 |
| EOF-206 | 4.83 | 33,251 |
| EOF-225 | 4.78 | 12,156 |
| EOF-242 | 5.28 | 75,945 |
| EOF-251 | 5.63 | 39,810 |
| EOF-254 | 5.77 | 30,205 |
| EOF-263 | 6.45 | 107,728 |
| EOF-267 | 5.52 | 55,278 |
| EOF-342 | 6.10 | 127,560 |
| EOF-349 | 6.63 | 112,864 |
| EOF-350 | 6.35 | 110,931 |
| EOF-352 | 5.84 | 105,012 |
| EOF-361 | 5.45 | 53,541 |
| EOF-366 | 5.89 | 44,569 |
| EOF-370 | 4.73 | 38,321 |
| EOF-371 | 5.45 | 35,693 |
| EOF-372 | 5.68 | 35,253 |
| EOF-376 | 5.40 | 28,445 |
| EOF-383 | 7.56 | 21,761 |
| EOF-387 | 4.57 | 13,644 |
| EOF-389 | 4.98 | 12,548 |
| EOF-400 | 5.10 | 63,380 |
| EOF-415 | 4.81 | 15,391 |
| EOF-419 | 5.96 | 10,109 |
| EOF-433 | 6.03 | 79,662 |
| EOF-435 | 6.90 | 78,821 |
| EOF-444 | 5.89 | 42,782 |
| EOF-460 | 4.69 | 11,984 |
| EOF-482 | 6.04 | 128,802 |
| EOF-489 | 5.00 | 120,241 |
| EOF-490 | 6.55 | 111,664 |
| EOF-493 | 5.08 | 107,091 |
| EOF-501 | 5.46 | 77,552 |
| EOF-510 | 6.16 | 62,248 |
| EOF-513 | 6.93 | 59,686 |
| EOF-523 | 4.95 | 53,870 |
| EOF-526 | 6.79 | 51,978 |
| EOF-527 | 5.84 | 50,081 |
| EOF-532 | 6.69 | 47,218 |
| EOF-533 | 6.99 | 45,426 |
| EOF-534 | 5.29 | 44,549 |
| EOF-536 | 6.61 | 42,351 |
| EOF-544 | 4.66 | 35,091 |
| EOF-546 | 4.66 | 34,343 |
| EOF-550 | 6.73 | 31,505 |
| EOF-552 | 5.59 | 30,717 |
| EOF-553 | 5.76 | 30,422 |
| EOF-554 | 5.34 | 29,967 |
| EOF-556 | 5.61 | 27,964 |
| EOF-560 | 7.57 | 25,483 |
| EOF-562 | 5.61 | 23,876 |
| EOF-574 | 4.65 | 18,637 |
| EOF-578 | 4.83 | 16,715 |
| EOF-582 | 4.53 | 14,162 |
| EOF-584 | 5.55 | 12,556 |
| EOF-609 | 5.54 | 52,543 |
| EOF-611 | 4.84 | 48,949 |
| EOF-612 | 4.91 | 48,777 |
| EOF-636 | 4.52 | 15,011 |
| EOF-665 | 5.60 | 35,566 |
| EOF-711 | 5.09 | 29,751 |
| EOF-713 | 4.93 | 27,497 |
| EOF-721 | 5.28 | 11,456 |
| EOF-724 | 5.79 | 42,413 |

The EOFs can be classified into two groups, i.e. those that are decreased and those that are increased in cells overexpressing ErbB2. The first group consists of EOFs that are significantly decreased in cell lines moderately or highly overexpressing ErbB2 compared to a control cell line. These EOFs are provided in Lists I-II and are described by apparent molecular weight (MW) and isoelectric point (pI) as provided in Table I. Preferred EOFs that are decreased in ErbB2 overexpressing cell lines as compared with normal cell lines include: EOF-86, EOF-106, EOF-183 and EOF-201.

List I—EOFs Decreased in Cell Lines Moderately Overexpressing ErbB2: EOF-34, EOF-35, EOF-44, EOF-57, EOF-61, EOF-63, EOF-74, EOF-75, EOF-81, EOF-83, EOF-84, EOF-86, EOF-87, EOF-94, EOF-99, EOF-106, EOF-110, EOF-120, EOF-133, EOF-135, EOF-136, EOF-137, EOF-139, EOF-149, EOF-153, EOF-156, EOF-166, EOF-167, EOF-182, EOF-183, EOF-188, EOF-196, EOF-197, EOF-201, EOF-225, EOF-242, EOF-254, EOF-263, EOF-267, EOF-342, EOF-350, EOF-370, EOF-372, EOF-383, EOF-387, EOF-415, EOF-419, EOF-433, EOF-435, EOF-444, EOF-460.

List II—EOFs Decreased in Cell Lines Highly Overexpressing ErbB2: EOF-342, EOF-350, EOF-383, EOF-415, EOF-419, EOF-433, EOF-435, EOF-444, EOF-460, EOF-489, EOF-490, EOF-493, EOF-526, EOF-532, EOF-544, EOF-546, EOF-552, EOF-553, EOF-554, EOF-584, EOF-611, EOF-612, EOF-636, EOF-711, EOF-713, EOF-721, EOF-724.

The second group consists of EOFs that are significantly increased in cell lines moderately or highly overexpressing ErbB2 compared to a control cell line. These EOFs are provided in Lists III-IV and can be described by apparent molecular weight (MW) and isoelectric point (pI) as provided in Table I. Preferred EOFs that are increased in ErbB2 overexpressing cell lines as compared with normal cell lines include: EOF-163, EOF-396, EOF-483, EOF-540, EOF-630, EOF-634, EOF-683 and EOF-693.

List III—EOFs Increased in Cell Lines Moderately Overexpressing ErbB2: EOF-48, EOF-51, EOF-66, EOF-67, EOF-70, EOF-72, EOF-73, EOF-76, EOF-76, EOF-80, EOF-90, EOF-102, EOF-104, EOF-107, EOF-111, EOF-129, EOF-138, EOF-145, EOF-163, EOF-206, EOF-251, EOF-349, EOF-352, EOF-361, EOF-366, EOF-371, EOF-376, EOF-389, EOF-396, EOF-400.

List IV—EOFs Increased in Cell Lines Highly Overexpressing ErbB2: EOF-349, EOF-352, EOF-361, EOF-366, EOF-370, EOF-371, EOF-372, EOF-376, EOF-387, EOF-389, EOF-396, EOF-400, EOF-482, EOF-483, EOF-501, EOF-510, EOF-513, EOF-523, EOF-527, EOF-533, EOF-534, EOF-536, EOF-550, EOF-556, EOF-560, EOF-562, EOF-574, EOF-578, EOF-582, EOF-609, EOF-630, EOF-634, EOF-665, EOF-683, EOF-693.

For any given EOF, the signal obtained upon analyzing tissue or body fluid from subjects having ErbB2 related cancer relative to the signal obtained upon analyzing tissue or body fluid from subjects free from ErbB2 related cancer will depend upon the particular analytical protocol and detection technique that is used. Accordingly, the present invention contemplates that each laboratory will, based on the present description, establish a reference range for each EOF in subjects free from ErbB2 related cancer according to the analytical protocol and detection technique in use, as is conventional in the diagnostic art. Preferably, at least one control positive tissue or body fluid sample from a subject known to have ErbB2 related cancer or at least one control negative tissue or body fluid sample from a subject known to be free from ErbB2 related cancer (and more preferably both positive and negative control samples) are included in each batch of test samples analyzed. In one embodiment, the level of expression of a feature is determined relative to a background value, which is defined as the level of signal obtained from a proximal region of the image that (a) is equivalent in area to the particular feature in question; and (b) contains no discernable protein feature.

In a preferred embodiment, the signal associated with an EOF in the tissue or body fluid of a subject (e.g., a subject suspected of having or known to have ErbB2 related cancer is normalised with reference to one or more Expression Reference Features (ERF)s detected in the same 2D gel. As will be apparent to one of ordinary skill in the art, such ERFs may readily be determined by comparing different samples using the Preferred Technology. Suitable ERFs include (but are not limited to) those described in Table II.

TABLE II
Expression Reference Features

| ERF | pI | MW (Da) |
|---|---|---|
| ERF-1 | 5.3 | 61,960 |
| ERF-2 | 5.4 | 57,275 |
| ERF-3 | 6.3 | 55,702 |
| ERF-4 | 6.6 | 53,462 |
| ERF-5 | 5.3 | 31,240 |

As those of skill in the art will readily appreciate, the apparent MW and pI of a given feature or protein isoform will vary to some extent depending on the precise protocol used for each step of the 2D electrophoresis and for landmark matching (as described in section 6.1.9 infra). As used herein, the terms “MW” and “pI” are defined, respectively, to mean the apparent molecular weight and the apparent isoelectric point of a feature or protein isoform as measured in exact accordance with the Reference Protocol identified in Section 6 below. When the Reference Protocol is followed and when samples are run in duplicate or a higher number of replicates, variation in the measured mean pI of an EOF or EOPI is typically less than 3% and variation in the measured mean MW of an EOF or EOPI is typically less than 5%. Where the skilled artisan wishes to deviate from the Reference Protocol, calibration experiments should be performed to compare the MW and pI for each EOF or EOPI as detected (a) by the Reference Protocol and (b) by the deviant protocol.

EOFs can be used for detection, diagnosis, or monitoring of ErbB2 related cancer, or for identifying patients most likely to respond to a specific therapeutic treatment, or for drug development. In one embodiment of the invention, a first sample of body fluid from a subject (e.g., a subject suspected of having ErbB2 related cancer) is analyzed by 2D electrophoresis for quantitative detection of one or more of the EOFs as defined in Lists I and II and described in Table I. A decreased abundance of said one or more of these EOFs in the first sample from the subject relative to a second sample from a subject or subjects free from ErbB2 related cancer (e.g., a control sample or a previously determined reference range) indicates the presence of ErbB2 related cancer.

In another embodiment of the invention, a first sample of body fluid from a subject is analyzed by 2D electrophoresis for the quantitative detection of one or more of the EOFs as defined in Lists III and IV and described in Table I. An increased abundance of said one or more EOFs in the first sample from the subject relative to a second sample from a subject or subjects free from ErbB2 related cancer (e.g., a control sample or a previously determined reference range) indicates the presence of ErbB2 related cancer.

In yet another embodiment, a first sample of tissue or body fluid from a subject is analyzed by 2D electrophoresis for quantitative detection of (a) one or more EOFs or any combination of them, whose decreased abundance indicates the presence of ErbB2 related cancer, i.e., the EOFs as defined in Lists I and II; and (b) one or more EOFs or any combination of them, whose increased abundance indicates the presence of ErbB2 related cancer i.e., EOFs as defined in Lists III and IV.

In yet another embodiment of the invention, a first sample of tissue or body fluid from a subject is analyzed by 2D electrophoresis for quantitative detection of one or more of the EOFs as defined in Lists I, II, III or IV and described in Table I; wherein the ratio of the one or more EOFs relative to an Expression Reference Feature (ERF) indicates whether ErbB2 related cancer is present. In a specific embodiment, a decrease in one or more EOF/ERF ratios in a first sample relative to the EOF/ERF ratios in a second sample or a reference range indicates the presence of ErbB2 related cancer; i.e. the EOFs as defined in Lists I and II are suitable EOFs for this purpose. In another specific embodiment, an increase in one or more EOF/ERF ratios in a first sample relative to the EOF/ERF ratios in a second sample or a reference range indicates the presence of ErbB2 related cancer; the EOFs as defined in Lists III and IV are suitable EOFs for this purpose.

In a further embodiment of the invention, a first sample of tissue or body fluid from a subject is analyzed by 2D electrophoresis for quantitative detection of (a) one or more EOFs, or any combination of them, whose decreased EOF/ERF ratio(s) in a first sample relative to the EOF/ERF ratio(s) in a second sample indicates the presence of ErbB2 related cancer, i.e., the EOFs as defined in Lists I and II; (b) one or more EOFs, or any combination of them, whose increased EOF/ERF ratio(s) in a first sample relative to the EOF/ERF ratio(s) in a second sample indicates the presence of ErbB2 related cancer, i.e., the EOFs as defined in Lists III and IV.

In a preferred embodiment, tissue or body fluid from a subject is analyzed for quantitative detection of a plurality of EOFs.

ErbB2 Overexpression-Associated Protein Isoforms (EOPIs)

In another aspect of the invention, a sample of body fluid from a subject, preferably a living subject, is analyzed for quantitative detection of one or more ErbB2 Overexpression-Associated Protein Isoforms (EOPIs) for screening or diagnosis of ErbB2 related cancer, to monitor the effectiveness of ErbB2 related cancer therapy, for identifying patients most likely to respond to a particular therapeutic treatment or for drug development. As is well known in the art, a given protein may be expressed as variants that differ in their amino acid composition (e.g. as a result of alternative mRNA or premRNA processing, e.g. alternative splicing or limited proteolysis) or as a result of differential post-translational modification (e.g., glycosylation, phosphorylation, acylation), or both, so that proteins of identical amino acid sequence can differ in their pI, MW, or both. It follows that differential presence of a protein isoform does not require differential expression of the gene encoding the protein in question. As used herein, the term “ErbB2 Overexpression-Associated Protein Isoform” refers to a polypeptide that is differentially present in a first sample of body fluid from a subject having ErbB2 related cancer compared with second sample from a subject free from ErbB2 related cancer.

The EOPIs have been identified by amino acid sequencing of EOFs. EOPIs were isolated, subjected to proteolysis, and analyzed by mass spectrometry using the methods and apparatus of the Preferred Technology. One skilled in the art can identify sequence information from proteins analyzed by mass spectrometry and/or tandem mass spectrometry using various spectral interpretation methods and database searching tools. Examples of some of these methods and tools can be found at the Swiss Institute of Bioinformatics web site at http://www.expasy.com/, and the European Molecular Biology Laboratory web site at http://www.narrador.embl-heidelberg.de/GroupPages/PageLink/peptidesearchpage.html. Identification of EOPIs was performed primarily using the SEQUEST search program (Eng et al., 1994, J. Am. Soc. Mass Spectrom. 5:976-989) and the method described in PCT/GB01/04034.

The amino acid sequences of peptides produced from these EOPIs by proteolysis using trypsin and identified by tandem mass spectrometry and database searching as described in the Examples, infra are listed in Table III, in addition to their corresponding pIs and MWs. Preferred EOPIs of the invention are EOPI-19, EOPI-22, EOPI-34, EOPI-59, EOPI-60, EOPI-62, EOPI-63, EOPI-92, EOPI-95, EOPI-115 and EOPI-125.

TABLE III
EOPIs Identified In Overexpressing ErbB2 Cells

| | | | | | |
|---|---|---|---|---|---|
| Feature | Is form | | | Amino Acid sequenc of | |
| (EOF) | (EOPI) | pl | MW (Da) | Tryptic digest | SEQ ID |

| | | | | | |
|---|---|---|---|---|---|
| EOF-86 | EOPI-19 | 6.16 | 47,684 | TIIQNPTDQQK | SEQ ID 232 |
| | | | | ITSGPFEPDLYK | SEQ ID 134 |
| EOF-106 | EOPI-22 | 5.08 | 40,638 | EALEVDWSSEK | SEQ ID 62 |
| | | | | AQNLNPMVDVK | SEQ ID 39 |
| | | | | EEAGGGISEEEAAQYDR | SEQ ID 65 |
| EOF-163 | EOPI-92 | 4.74 | 14,222 | ENVVQSVTSVAEK | SEQ ID 5 |
| | | | | TVEEAENIAVTSGVVR | SEQ ID 12 |
| EOF-183 | EOPI-95 | 5.04 | 105,577 | EDQTEYLEER | SEQ ID 64 |
| | | | | LGIHEDSQNR | SEQ ID 153 |
| | | | | GVVDSEDLPLNISR | SEQ ID 104 |
| EOF-201 | EOPI-34 | 6.37 | 43,168 | TVQIEASTVEIEER | SEQ ID 239 |
| | | | | TIISYIDEQFER | SEQ ID 233 |
| | | | | ILDEIEEHNIK | SEQ ID 122 |
| EOF-396 | EOPI-115 | 5.78 | 104,459 | YTYEHDPITK | SEQ ID 267 |
| | | | | ELSAVTFPDIIR | SEQ ID 76 |
| EOF-483 | EOPI-125 | 5.39 | 127,995 | KPLLESGTLGTK | SEQ ID 137 |
| | | | | NEEDAAELVALAQAVNAR | SEQ ID 181 |
| | | | | LAGTQPLEVLEAVQR | SEQ ID 139 |
| | | | | AENYDIPSADR | SEQ ID 26 |
| EOF-630 | EOPI-59 | 4.98 | 21,851 | VLGMTLIQK | SEQ ID 253 |
| | | | | SLDFYTR | SEQ ID 212 |
| | | | | RFEELGVK | SEQ ID 204 |
| | | | | DFLLQQTMLR | SEQ ID 51 |
| EOF-634 | EOPI-60 | 5.05 | 16,573 | EDGQEYAQVIK | SEQ ID 63 |
| | | | | AYGELPEHAK | SEQ ID 47 |
| EOF-683 | EOPI-62 | 5.45 | 13,968 | NSILAQVLDQSAR | SEQ ID 188 |
| | | | | LSNLALVKPEK | SEQ ID 171 |
| EOF-693 | EOPI-63 | 4.99 | 10,445 | LQDAEIAR | SEQ ID 167 |
| | | | | LMEDLDR | SEQ ID 162 |
| EOF-34 | EOPI-67 | 6.29 | 113,362 | SEDYVDIVQGNR | SEQ ID 18 |
| | | | | IVGPEENLSQAEAR | SEQ ID 8 |
| EOF-35 | EOPI-1 | 6.51 | 110,562 | ILIEQNR | SEQ ID 6 |
| | | | | IVGPEENLSQAEAR | SEQ ID 8 |
| | | | | SEDYVDIVQGNR | SEQ ID 18 |
| EOF-44 | EOPI-2 | 6.44 | 85,990 | SQLLSYIDR | SEQ ID 222 |
| | | | | SPQNQYPAELMR | SEQ ID 220 |
| | | | | DVLDVYIEHR | SEQ ID 56 |
| EOF-48 | EOPI-3 | 4.93 | 80,273 | VTHAVVTVPAYFNDAQR | SEQ ID 258 |
| | | | | SDIDEIVLVGGSTR | SEQ ID 209 |
| EOF-51 | EOPI-4 | 6.61 | 74,208 | AQQELEEQTR | SEQ ID 40 |
| | | | | APDFVFYAPR | SEQ ID 36 |
| EOF-57 | EOPI-70 | 5.16 | 68,895 | LSPEELLLR | SEQ ID 172 |
| | | | | QFVTATDVVR | SEQ ID 196 |
| | | | | FSLVGIGGQDLNEGNR | SEQ ID 87 |
| | | | | GDEEGVPAVVIDMSGLR | SEQ ID 96 |
| EOF-61 | EOPI-71 | 7.73 | 61,037 | VAGALAEAGVGLEEIAK | SEQ ID 243 |
| | | | | LEQPDPGAVAAAAILR | SEQ ID 147 |
| EOF-63 | EOPI-5 | 5.02 | 58,032 | MFGGPGTASR | SEQ ID 175 |
| | | | | TYSLGSALR | SEQ ID 242 |
| | | | | SLYASSPGGVYATR | SEQ ID 216 |
| EOF-66 | EOPI-72 | 5.47 | 56,144 | SMQDVVEDFK | SEQ ID 217 |
| | | | | VDALNDEINFLR | SEQ ID 244 |
| | | | | SAYGGPVGAGIR | SEQ ID 208 |
| | | | | GQLEALQVDGGR | SEQ ID 101 |
| | | | | AKQEELEAALQR | SEQ ID 33 |
| EOF-67 | EOPI-6 | 6.92 | 56,452 | VFVEEQVYSEFVR | SEQ ID 251 |
| | | | | LLHQLADLVER | SEQ ID 156 |
| EOF-70 | EOPI-7 | 4.80 | 54,791 | AGLENTVAETECR | SEQ ID 29 |
| | | | | MIGFPSSAGSVSPR | SEQ ID 176 |
| EOF-70 | EOPI-8 | 4.80 | 54,791 | MIGFPSSAGSVSPR | SEQ ID 176 |
| EOF-72 | EOPI-9 | 5.58 | 52,905 | LEGLTDEINFLR | SEQ ID 145 |
| | | | | LSELEAALQR | SEQ ID 170 |
| | | | | SNMDNMFESYINNLR | SEQ ID 219 |
| | | | | LEAELGNMQGLVEDFK | SEQ ID 142 |
| EOF-73 | EOPI-11 | 4.76 | 53,395 | ISEQFTAMFR | SEQ ID 132 |
| | | | | AILVDLEPGTMDSVR | SEQ ID 32 |
| EOF-74 | EOPI-12 | 5.27 | 52,894 | LEAPLEELR | SEQ ID 143 |
| | | | | DPLELFR | SEQ ID 54 |
| EOF-75 | EOPI-13 | 5.16 | 51,876 | TVLSGGTTMYPGIADR | SEQ ID 238 |
| | | | | GYSFTTTAER | SEQ ID 106 |
| | | | | DLTDYLMK | SEQ ID 53 |
| EOF-76 | EOPI-14 | 4.83 | 52,196 | LAVNMVPFPR | SEQ ID 140 |
| | | | | AILVDLEPGTMDSVR | SEQ ID 32 |
| EOF-76 | EOPI-15 | 4.83 | 52,196 | MIGFPSSAGSVSPR | SEQ ID 176 |
| | | | | ITMQNLNDR | SEQ ID 133 |
| EOF-80 | EOPI-16 | 4.67 | 51,143 | FVSSGSGGGYGGGMR | SEQ ID 92 |
| EOF-81 | EOPI-17 | 5.17 | 49,168 | GYSFTTTAER | SEQ ID 106 |
| | | | | SYELPDGQVITIGNER | SEQ ID 225 |
| | | | | QEYDESGPSIVHR | SEQ ID 194 |
| | | | | AGFAGDDAPR | SEQ ID 28 |
| EOF-81 | EOPI-18 | 5.17 | 49,168 | SENEEFVEVGR | SEQ ID 210 |
| | | | | GAISAEVYTEEDAASYVR | SEQ ID 94 |
| EOF-83 | EOPI-73 | 5.06 | 49,351 | DSYLILETLPTEYDSR | SEQ ID 3 |
| EOF-84 | EOPI-74 | 4.77 | 49,233 | FADLSEAANR | SEQ ID 80 |
| | | | | LQEEMLQR | SEQ ID 168 |
| | | | | VELQELNDR | SEQ ID 249 |
| EOF-87 | EOPI-75 | 4.61 | 47,205 | TFDQLTPEESK | SEQ ID 231 |
| | | | | EQFVEFR | SEQ ID 78 |
| EOF-90 | EOPI-20 | 5.39 | 44,908 | AQYDELAR | SEQ ID 41 |
| | | | | QSVENDIHGLR | SEQ ID 201 |
| | | | | TVQSLEIDLDSMR | SEQ ID 240 |
| | | | | QAQEYEALLNIK | SEQ ID 193 |
| | | | | SLGSVQAPSYGAR | SEQ ID 215 |
| | | | | KVIDDTNITR | SEQ ID 138 |
| | | | | STFSTNYR | SEQ ID 224 |
| EOF-94 | EOPI-77 | 6.13 | 44,019 | VNIKPQVDR | SEQ ID 256 |
| | | | | YPQLLPGIR | SEQ ID 265 |
| EOF-94 | EOPI-78 | 6.13 | 44,019 | ELAEQLGLSTGEK | SEQ ID 74 |
| | | | | LPGELEPVQATQNK | SEQ ID 166 |
| EOF-99 | EOPI-80 | 5.40 | 42,809 | LDGEASINNR | SEQ ID 141 |
| | | | | QTFGYGAR | SEQ ID 202 |
| | | | | GIPAPEEER | SEQ ID 98 |
| EOF-102 | EOPI-21 | 5.39 | 42,227 | DVQGTDASLDEELDR | SEQ ID 58 |
| | | | | APVPGTPDSLSSGSSR | SEQ ID 37 |
| EOF-104 | EOPI-81 | 6.72 | 41,786 | LRDDTELR | SEQ ID 169 |
| | | | | ASSVVVSGTPIR | SEQ ID 42 |
| EOF-107 | EOPI-23 | 5.74 | 40,619 | IIELPFQNK | SEQ ID 117 |
| | | | | GVALSNVVHK | SEQ ID 102 |
| | | | | GDTANEIGQVLHFENVK | SEQ ID 97 |
| | | | | ELETVDFK | SEQ ID 75 |
| | | | | DELNADHPFIYIIR | SEQ ID 50 |
| | | | | HIFSEDTSDFSGMSETK | SEQ ID 108 |
| | | | | DVPFGFQTVTSDVNK | SEQ ID 57 |
| EOF-110 | EOPI-24 | 6.16 | 39,933 | QPQVAELLAEAR | SEQ ID 198 |
| | | | | EEFGAEPELAVSAPGR | SEQ ID 66 |
| EOF-111 | EOPI-82 | 4.77 | 40,050 | VEVTEFEDIK | SEQ ID 250 |
| | | | | EFHLNESGDPSSK | SEQ ID 69 |
| EOF-120 | EOPI-84 | 5.77 | 36,187 | ILIEELR | SEQ ID 124 |
| | | | | AGVDPLVPLR | SEQ ID 30 |
| | | | | SLGLSLSGGDQEDAGR | SEQ ID 214 |
| EOF-129 | EOPI-85 | 4.61 | 31,848 | NLLSVAYK | SEQ ID 186 |
| | | | | YLAEFATGNDR | SEQ ID 262 |
| EOF-133 | EOPI-86 | 5.16 | 31,009 | FSWFAGEK | SEQ ID 89 |
| | | | | LLLEFTDTSYEEK | SEQ ID 158 |
| | | | | VDIIENQVMDFR | SEQ ID 245 |
| EOF-135 | EOPI-87 | 5.60 | 30,301 | IIVLGLLPR | SEQ ID 121 |
| EOF-136 | EOPI-25 | 5.14 | 29,942 | SNFGYNIPLK | SEQ ID 19 |
| EOF-136 | EOPI-25 | 5.14 | 29,942 | AVENSSTAIGIR | SEQ ID 1 |
| EOF-137 | EOPI-26 | 5.39 | 29,939 | SSYYMIGEQK | SEQ ID 223 |
| | | | | HSAESQILK | SEQ ID 112 |
| | | | | FSSLPLGR | SEQ ID 88 |
| EOF-138 | EOPI-88 | 6.10 | 29,850 | NPVPAQDEALYQQLLR | SEQ ID 187 |
| | | | | GVPFTLTTVDTR | SEQ ID 103 |
| | | | | QAPIPAELR | SEQ ID 192 |
| EOF-139 | EOPI-89 | 5.21 | 29,877 | AVENSSTAIGIR | SEQ ID 1 |
| EOF-145 | EOPI-90 | 5.04 | 28,469 | TLLGDGPVVTDPK | SEQ ID 234 |
| | | | | YVQHTYR | SEQ ID 268 |
| | | | | LNYKPPPQK | SEQ ID 164 |
| EOF-149 | EOPI-27 | 5.54 | 23,266 | PPYTVVYFPVR | SEQ ID 191 |
| | | | | MLLADQGQSWK | SEQ ID 178 |
| | | | | FQDGDLTLYQSNTILR | SEQ ID 85 |
| | | | | EEVVTVETWQEGSLK | SEQ ID 68 |
| | | | | AFLASPEYVNLPINGNGK | SEQ ID 27 |
| EOF-153 | EOPI-91 | 5.45 | 21,277 | IDYIAGLDSR | SEQ ID 114 |
| | | | | SFPDFPTPGVVFR | SEQ ID 211 |
| EOF-156 | EOPI-28 | 5.06 | 19,901 | VEEAEPEEFVVEK | SEQ ID 247 |
| | | | | IIGATDSSGELMFLMK | SEQ ID 118 |
| EOF-166 | EOPI-29 | 4.96 | 12,052 | LPDGYEFK | SEQ ID 165 |
| | | | | LNLEAINYMAADGDFK | SEQ ID 163 |
| EOF-167 | EOPI-94 | 5.26 | 12,062 | SQGGEPTYNVAVGR | SEQ ID 221 |
| EOF-182 | EOPI-30 | 5.10 | 106,575 | GVVDSEDLPLNISR | SEQ ID 104 |
| | | | | IDIIPNPQER | SEQ ID 113 |
| EOF-188 | EOPI-96 | 6.29 | 69,345 | LFELEEQDLFR | SEQ ID 151 |
| | | | | GYDFPAVLR | SEQ ID 105 |
| EOF-196 | EOPI-31 | 5.55 | 50,803 | LSVLGAITSVQQR | SEQ ID 173 |
| | | | | ILYLTPEQEK | SEQ ID 127 |
| EOF-196 | EOPI-32 | 5.55 | 50,803 | MLEQLDMR | SEQ ID 177 |
| | | | | ILVATNLFGR | SEQ ID 126 |
| EOF-197 | EOPI-33 | 5.59 | 50,859 | QQLEELAR | SEQ ID 200 |
| | | | | EGGGNNLYGEEMVQALK | SEQ ID 71 |
| EOF-206 | EOPI-97 | 4.83 | 33,251 | VLTEIIASR | SEQ ID 254 |
| | | | | YMTISGFQIEETIDR | SEQ ID 264 |
| | | | | GAGTDDHTLIR | SEQ ID 93 |
| EOF-225 | EOPI-98 | 4.78 | 12,156 | LPDGYEFK | SEQ ID 165 |
| | | | | LNLEAINYMAADGDFK | SEQ ID 163 |
| EOF-242 | EOPI-35 | 5.28 | 75,945 | FEELNADLFR | SEQ ID 82 |
| EOF-251 | EOPI-99 | 5.63 | 39,810 | IIELPFQNK | SEQ ID 117 |
| | | | | DVPFGFQTVTSDVNK | SEQ ID 57 |
| EOF-254 | EOPI-100 | 5.77 | 30,205 | LLPDDPYEK | SEQ ID 160 |
| EOF-263 | EOPI-102 | 6.45 | 107,728 | HFSVEGQLEFR | SEQ ID 107 |
| | | | | IDIIPNPQER | SEQ ID 113 |
| | | | | GVVDSEDLPLNISR | SEQ ID 104 |
| EOF-267 | EOPI-104 | 5.52 | 55,278 | NFIAQGPYENR | SEQ ID 182 |
| | | | | IPQSTLSEFYPR | SEQ ID 130 |
| EOF-342 | EOPI-105 | 6.10 | 127,560 | TEEGPTLSYGR | SEQ ID 228 |
| EOF-349 | EOPI-106 | 6.63 | 112,864 | LTHLHEGLPVK | SEQ ID 17 |
| | | | | IVGPEENLSQAEAR | SEQ ID 8 |
| | | | | SEDYVDIVQGNR | SEQ ID 18 |
| EOF-350 | EOPI-107 | 6.35 | 110,931 | ILIEQNR | SEQ ID 6 |
| | | | | LTHLHEGLPVK | SEQ ID 17 |
| | | | | SEDYVDIVQGNR | SEQ ID 18 |
| EOF-352 | EOPI-108 | 5.84 | 105,012 | YTYEHDPITK | SEQ ID 267 |
| | | | | ELSAVTFPDIIR | SEQ ID 76 |
| | | | | TELISVSEVHPSR | SEQ ID 230 |
| EOF-361 | EOPI-36 | 5.45 | 53,541 | TEISEMNR | SEQ ID 229 |
| | | | | SNMDNMFESYINNLR | SEQ ID 219 |
| EOF-366 | EOPI-110 | 5.89 | 44,569 | FGGNPGGFGNQGGFGNSR | SEQ ID 83 |
| | | | | FTEYETQVK | SEQ ID 91 |
| EOF-370 | EOPI-37 | 4.73 | 38,321 | VDNDENEHQLSLR | SEQ ID 246 |
| | | | | MTDQEAIQDLWQWR | SEQ ID 179 |
| EOF-371 | EOPI-111 | 5.45 | 35,693 | LLLNNDNLLR | SEQ ID 159 |
| EOF-371 | EOPI-112 | 5.45 | 35,693 | IMEGPAFNFLDAPAVR | SEQ ID 128 |
| | | | | DAINQGMDEELER | SEQ ID 49 |
| EOF-372 | EOPI-38 | 5.68 | 35,253 | SADTLWDIQK | SEQ ID 207 |
| | | | | MVVESAYEVIK | SEQ ID 180 |
| | | | | IVVVTAGVR | SEQ ID 136 |
| | | | | LIAPVAEEEATVPNNK | SEQ ID 154 |
| EOF-376 | EOPI-39 | 5.40 | 28,445 | QFLSETEK | SEQ ID 195 |
| | | | | LGFEDGSVLK | SEQ ID 152 |
| EOF-383 | EOPI-113 | 7.56 | 21,761 | NVIGLQMGTNR | SEQ ID 189 |
| | | | | QMEQISQFLQAAER | SEQ ID 197 |
| | | | | GASQAGMTGYGMPR | SEQ ID 95 |
| EOF-387 | EOPI-40 | 4.57 | 13,644 | EAFQLFDR | SEQ ID 61 |
| | | | | DQGTYEDYVEGLR | SEQ ID 55 |
| | | | | ALGQNPTNAEVLK | SEQ ID 34 |
| EOF-389 | EOPI-114 | 4.98 | 12,548 | LPDGYEFK | SEQ ID 165 |
| | | | | LNLEAINYMAADGDFK | SEQ ID 163 |
| EOF-400 | EOPI-116 | 5.10 | 63,380 | LSPEELLLR | SEQ ID 172 |
| | | | | FSLVGIGGQDLNEGNR | SEQ ID 87 |
| | | | | QFVTATDVVR | SEQ ID 196 |
| | | | | GDEEGVPAVVIDMSGLR | SEQ ID 96 |
| EOF-415 | EOPI-117 | 4.81 | 15,391 | INPDGSQSVVEVPYAR | SEQ ID 129 |
| | | | | NGESSELDLQGIR | SEQ ID 184 |
| EOF-419 | EOPI-118 | 5.96 | 10,109 | TEFLSFMNTELAAFTK | SEQ ID 11 |
| | | | | ISSPTETER | SEQ ID 7 |
| EOF-433 | EOPI-119 | 6.03 | 79,662 | LEQYTSAIEGTK | SEQ ID 148 |
| EOF-435 | EOPI-121 | 6.90 | 78,821 | LLDYVPIGPR | SEQ ID 155 |
| | | | | LFADAEEEQR | SEQ ID 150 |
| | | | | NFSGAELEGLVR | SEQ ID 183 |
| EOF-435 | EOPI-120 | 6.90 | 78,821 | TYDSYLGDDYVR | SEQ ID 241 |
| EOF-444 | EOPI-122 | 5.89 | 42,782 | FGGNPGGFGNQGGFGNSR | SEQ ID 83 |
| | | | | FTEYETQVK | SEQ ID 91 |
| EOF-460 | EOPI-123 | 4.69 | 11,984 | EHALTSGTIK | SEQ ID 72 |
| | | | | EIPSHVLSK | SEQ ID 73 |
| EOF-482 | EOPI-124 | 6.04 | 128,802 | LLAVAATAPPDAPNR | SEQ ID 9 |
| | | | | ELTPQVVSAAR | SEQ ID 15 |
| EOF-489 | EOPI-126 | 5.00 | 120,241 | DIPNENEAQFQIR | SEQ ID 2 |
| EOF-490 | EOPI-41 | 6.55 | 111,664 | LLLCNPR | SEQ ID 157 |
| EOF-493 | EOPI-42 | 5.08 | 107,091 | EDQTEYLEER | SEQ ID 64 |
| | | | | HIYYITGETK | SEQ ID 109 |
| | | | | GVVDSEDLPLNISR | SEQ ID 104 |
| EOF-501 | EOPI-127 | 5.46 | 77,552 | VEIIANDQGNR | SEQ ID 248 |
| | | | | DAGVIAGLNVLR | SEQ ID 48 |
| | | | | TTPSYVAFTDTER | SEQ ID 237 |
| | | | | AQIHDLVLVGGSTR | SEQ ID 38 |
| EOF-510 | EOPI-43 | 6.16 | 62,248 | AVAQALEVIPR | SEQ ID 44 |
| EOF-513 | EOPI-129 | 6.93 | 59,686 | HLIEQDFPGMR | SEQ ID 110 |
| | | | | LLPLEEHYR | SEQ ID 161 |
| | | | | ADQIETQQLMR | SEQ ID 22 |
| | | | | IINTPEVVR | SEQ ID 119 |
| EOF-523 | EOPI-44 | 4.95 | 53,870 | YLTVATVFR | SEQ ID 263 |
| | | | | ISEQFTAMFR | SEQ ID 132 |
| | | | | AILVDLEPGTMDSVR | SEQ ID 32 |
| EOF-526 | EOPI-45 | 6.79 | 51,978 | SYELQESNVR | SEQ ID 226 |
| | | | | RNEFLGELQK | SEQ ID 206 |
| | | | | VNMEDLR | SEQ ID 257 |
| | | | | SLFNYHDTR | SEQ ID 213 |
| EOF-527 | EOPI-46 | 5.84 | 50,081 | YSLVLELSDSGAFR | SEQ ID 266 |
| | | | | DYLLLVMEGTDDGR | SEQ ID 59 |
| | | | | GLFEVNPWK | SEQ ID 99 |
| | | | | LENGEIETIAR | SEQ ID 146 |
| EOF-532 | EOPI-130 | 6.69 | 47,218 | QVLEPSFR | SEQ ID 203 |
| | | | | ALIAAQYSGAQVR | SEQ ID 35 |
| EOF-533 | EOPI-47 | 6.99 | 45,426 | YEEIDNAPEER | SEQ ID 260 |
| | | | | AEAGDNLGALVR | SEQ ID 23 |
| EOF-534 | EOPI-48 | 5.29 | 44,549 | TVLSGGTTMYPGIADR | SEQ ID 238 |
| EOF-534 | EOPI-48 | 5.29 | 44,549 | GYSFTTTAER | SEQ ID 106 |
| EOF-536 | EOPI-131 | 6.61 | 42,351 | LEEGPPVTTVLTR | SEQ ID 144 |
| EOF-544 | EOPI-49 | 4.66 | 35,091 | LVIIESDLER | SEQ ID 174 |
| | | | | IQLVEEELDR | SEQ ID 131 |
| | | | | ATDAEADVASLNR | SEQ ID 43 |
| EOF-546 | EOPI-50 | 4.66 | 34,343 | LVIIESDLER | SEQ ID 174 |
| | | | | AELSEGQVR | SEQ ID 25 |
| EOF-550 | EOPI-133 | 6.73 | 31,505 | EQQILTLFR | SEQ ID 79 |
| | | | | EEIIGNGEQQYVYLK | SEQ ID 67 |
| EOF-552 | EOPI-51 | 5.59 | 30,717 | YDPPLEDGAMPSAR | SEQ ID 259 |
| | | | | RLPPQQIEK | SEQ ID 205 |
| | | | | LEVEANNAFDQYR | SEQ ID 149 |
| EOF-553 | EOPI-134 | 5.76 | 30,422 | EGVKFDESEKTK | SEQ ID 4 |
| | | | | ILDQGEDFPASEMTR | SEQ ID 269 |
| EOF-554 | EOPI-136 | 5.34 | 29,967 | DLQNVNITLR | SEQ ID 52 |
| | | | | FDAGELITQR | SEQ ID 81 |
| | | | | IFTSIGEDYDER | SEQ ID 116 |
| EOF-556 | EOPI-137 | 5.61 | 27,964 | TQNPMVTGTSVLGVK | SEQ ID 235 |
| | | | | FQTATVTEK | SEQ ID 86 |
| | | | | AIHSWLTR | SEQ ID 31 |
| | | | | QPVLSQTEAR | SEQ ID 199 |
| EOF-560 | EOPI-139 | 7.57 | 25,483 | NVPNWHR | SEQ ID 190 |
| | | | | HLTGEFEK | SEQ ID 111 |
| | | | | FNVWDTAGQEK | SEQ ID 84 |
| EOF-562 | EOPI-140 | 5.61 | 23,876 | PPYTVVYFPVR | SEQ ID 191 |
| | | | | FQDGDLTLYQSNTILR | SEQ ID 85 |
| | | | | MLLADQGQSWK | SEQ ID 178 |
| | | | | EEVVTVETWQEGSL | SEQ ID 68 |
| EOF-574 | EOPI-52 | 4.65 | 18,637 | FTDEEVDELYR | SEQ ID 90 |
| | | | | EAFNMIDQNR | SEQ ID 60 |
| EOF-578 | EOPI-53 | 4.83 | 16,715 | EPVDFEQWIEK | SEQ ID 77 |
| EOF-582 | EOPI-54 | 4.53 | 14,162 | ILDSVGIEADDDR | SEQ ID 123 |
| EOF-582 | EOPI-55 | 4.53 | 14,162 | EAFQLFDR | SEQ ID 61 |
| | | | | DQGTYEDYVEGLR | SEQ ID 55 |
| | | | | ALGQNPTNAEVLK | SEQ ID 34 |
| EOF-584 | EOPI-141 | 5.55 | 12,556 | QEGDTFYIK | SEQ ID 10 |
| | | | | VLGVNVMLR | SEQ ID 13 |
| EOF-609 | EOPI-56 | 5.54 | 52,543 | VLTLSDDLER | SEQ ID 255 |
| EOF-611 | EOPI-57 | 4.84 | 48,949 | IVIGMDVAASEFYR | SEQ ID 135 |
| | | | | IEEELGDEAR | SEQ ID 115 |
| EOF-612 | EOPI-58 | 4.91 | 48,777 | ADLEMQIENLK | SEQ ID 21 |
| | | | | NHEEEMNALR | SEQ ID 185 |
| EOF-636 | EOPI-143 | 4.52 | 15,011 | ADQFEYVMYGK | SEQ ID 14 |
| | | | | IEGDETSTEAATR | SEQ ID 16 |
| EOF-665 | EOPI-61 | 5.60 | 35,566 | VLALSVETDYTFPLAEK | SEQ ID 252 |
| | | | | TSFFQALGITTK | SEQ ID 236 |
| | | | | IIQLLDDYPK | SEQ ID 120 |
| | | | | GNVGFVFTK | SEQ ID 100 |
| EOF-711 | EOPI-144 | 5.09 | 29,751 | SNFGYNIPLK | SEQ ID 19 |
| | | | | VFQVEYAMK | SEQ ID 20 |
| | | | | AVENSSTAIGIR | SEQ ID 1 |
| EOF-713 | EOPI-64 | 4.93 | 27,497 | TDYMVGSYGPR | SEQ ID 227 |
| | | | | YIQHTYR | SEQ ID 261 |
| | | | | AEEYEFLTPVEEAPK | SEQ ID 24 |
| EOF-721 | EOPI-65 | 5.28 | 11,456 | EGFFTNGLTLGAK | SEQ ID 70 |
| | | | | AYELALYLR | SEQ ID 46 |
| | | | | SQGGEPTYNVAVGR | SEQ ID 221 |
| EOF-724 | EOPI-145 | 5.79 | 42,413 | GDTANEIGQVLHFENVK | SEQ ID 97 |
| | | | | DVPFGFQTVTSDVNK | SEQ ID 57 |

The EOPIs can be classified into two groups, i.e. those that are decreased and those that are increased in ErbB2 overexpressing cells. The first group comprises of EOPIs that are significantly decreased in cell lines that are moderately or highly overexpressing ErbB2 compared with control cell lines. These EOPIs are defined in Lists V-VI below, and the amino acid sequences of peptides produced from these EOPIs by proteolysis using trypsin and identified by tandem mass spectrometry and database searching are listed in Table III in addition to their corresponding pls and MWs. Preferred EOPIs of the invention that are decreased in ErbB2 overexpressing cell lines as compared with normal cell lines include: EOPI-19, EOPI-22, EOPI-34 and EOPI-95.

List V. EOPIs Decreased in Cell Lines Moderately Overexpressing ErbB2: EOPI-67, EOPI-1, EOPI-2, EOPI-70, EOPI-71, EOPI-5, EOPI-12, EOPI-13, EOPI-17, EOPI-18, EOPI-73, EOPI-74, EOPI-19, EOPI-75, EOPI-77, EOPI-78, EOPI-80, EOPI-22, EOPI-24, EOPI-84, EOPI-86, EOPI-87, EOPI-25, EOPI-26, EOPI-89, EOPI-27, EOPI-91, EOPI-28, EOPI-29, EOPI-94, EOPI-30, EOPI-95, EOPI-96, EOPI-31, EOPI-32, EOPI-33, EOPI-34, EOPI-98, EOPI-35, EOPI-100, EOPI-102, EOPI-104, EOPI-105, EOPI-107, EOPI-37, EOPI-38, EOPI-113, EOPI-40, EOPI-117, EOPI-118, EOPI-119, EOPI-121, EOPI-120, EOPI-122, EOPI-123.

List VI. EOPIs Decreased in Cell Lines Highly Overexpressing ErbB2 Isoform (EOPI): EOPI-105, EOPI-107, EOPI-113, EOPI-117, EOPI-118, EOPI-119, EOPI-121, EOPI-120, EOPI-122, EOPI-123, EOPI-126, EOPI-41, EOPI-42, EOPI-45, EOPI-130, EOPI-49, EOPI-50, EOPI-51, EOPI-134, EOPI-136, EOPI-141, EOPI-57, EOPI-58, EOPI-143, EOPI-144, EOPI-64, EOPI-65, EOPI-145.

The second group comprises EOPIs that are significantly increased in cell lines moderately or highly overexpressing ErbB2 as compared with control cell lines. These EOPIs are defined in Lists VII-VIII below, and the amino acid sequences of peptides produced from these EOPIs by proteolysis using trypsin and identified by tandem mass spectrometry and database searching are listed in Table III in addition to their corresponding pIs and MWs. Preferred EOPIs of the invention that are increased in ErbB2 overexpressing cell lines as compared with normal cell lines include: EOPI-59, EOPI-60, EOPI-62, EOPI-63, EOPI-92, EOPI-115, EOPI-125 and EOPI-132.

List VII. EOPIs Increased in Cell Lines Moderately Overexpressing ErbB2 Isoform (EOPI): EOPI-3, EOPI-4, EOPI-72, EOPI-6, EOPI-7, EOPI-8, EOPI-9, EOPI-11, EOPI-14, EOPI-15, EOPI-16, EOPI-20, EOPI-21, EOPI-81, EOPI-23, EOPI-82, EOPI-85, EOPI-88, EOPI-90, EOPI-92, EOPI-97, EOPI-99, EOPI-106, EOPI-108, EOPI-36, EOPI-110, EOPI-111, EOPI-112, EOPI-39, EOPI-114, EOPI-115, EOPI-116.

List VIII. EOPIs Increased in Cell Lines Highly Overexpressing ErbB2 Isoform (EOPI): EOPI-36, EOPI-37, EOPI-38, EOPI-39, EOPI-40, EOPI-43, EOPI-44, EOPI-46, EOPI-47, EOPI-48, EOPI-52, EOPI-53, EOPI-54, EOPI-55, EOPI-56, EOPI-59, EOPI-60, EOPI-61, EOPI-62, EOPI-63, EOPI-106, EOPI-108, EOPI-110, EOPI-111, EOPI-112, EOPI-114, EOPI-115, EOPI-116, EOPI-124, EOPI-125, EOPI-127, EOPI-129, EOPI-131, EOPI-133, EOPI-137, EOPI-139, EOPI-140.

As will be evident to one of skill in the art, based upon the present description, a given EOPI can be described according to the data provided for that EOPI in Tables III. The EOPI is a polypeptide comprising a peptide sequence described for that EOPI (preferably comprising a plurality of, more preferably all of, the peptide sequences described for that EOPI) and has a pI of about the value stated for that EOPI (preferably within 10%, more preferably within 5% still more preferably within 1% of the stated value) and has a MW of about the value stated for that EOPI (preferably within 10%, more preferably within 5%, still more preferably within 1% of the stated value).

In one embodiment, a first sample of body fluid from a subject is analyzed for quantitative detection of one or more of the EOPIs as defined in Lists V and VI, or any combination of them, wherein a decreased abundance of the EOPI or EOPIs (or any combination of them) in the first sample from the subject relative to the second sample from a subject or subjects free from ErbB2 related cancer (e.g., a control sample or a previously determined reference range) indicates the presence of ErbB2 related cancer.

In another embodiment of the invention, a first sample of body fluid from a subject is analyzed for quantitative detection of one or more of the EOPIs as defined in Lists VII and VIII, or any combination of them, wherein an increased abundance of the EOPI or EOPIs (or any combination of them) in the first sample from the subject relative to the second sample from a subject or subjects free from ErbB2 related cancer (e.g., a control sample or a previously determined reference range) indicates the presence of ErbB2 related cancer.

In a further embodiment, a first sample of body fluid from a subject is analyzed for quantitative detection of (a) one or more EOPIs, or any combination of them, whose decreased abundance indicates the presence of ErbB2 related cancer, i.e., the EOPIs as defined in Lists V and VI; and (b) one or more EOPIs, or any combination of them, whose increased abundance indicates the presence of ErbB2 related cancer, i.e., the EOPIs as defined in Lists VII and VIII.

In yet a further embodiment, a first sample of body fluid from a subject is analyzed for quantitative detection of one or more EOPIs and one or more previously known biomarkers of ErbB2 related cancer (e.g., candidate markers such as hypersensitive platelet glutamate receptors (Berk et al. Int Clin Psychopharmacol 199914, 199-122)). In accordance with this embodiment, the abundance of each EOPI and known biomarker relative to a control or reference range indicates whether a subject has ErbB2 related cancer.

Preferably, the abundance of an EOPI is normalized to an Expression Reference Protein Isoform (ERPI). ERPIs can be identified by partial amino acid sequencing of ERFs, which are described above, using the methods and apparatus of the Preferred Technology.

The EOPIs described herein include isoforms of known proteins where the isoforms were not previously known to be associated with ErbB2 related cancer. For each EOPI, the present invention additionally provides: (a) antibodies that bind to said EOPI, to said fragments, or both to said EOPI and to said fragments. Preferably the EOPI is in an isolated form, as used herein, an EOPI is “isolated” when it is present in a preparation that is substantially free of contaminating proteins, i.e., a preparation in which less than 10% (preferably less than 5%, more preferably less than 1%) of the total protein present is contaminating protein(s). A contaminating protein is a protein or protein isoform having a significantly different pI or MW from those of the isolated EOPI, as determined by 2D electrophoresis. As used herein, a “significantly different” pI or MW is one that permits the contaminating protein to be resolved from the EOPI on 2D electrophoresis, performed according to the Reference Protocol.

In one embodiment, an isolated polypeptide is provided, said polypeptide comprising a peptide with the amino acid sequence identified in Table III for an EOPI, said polypeptide having a pI and MW within 10% (preferably within 5%, more preferably within 1%) of the values identified in Table III for that EOPI.

The EOPIs of the invention can be qualitatively or quantitatively detected by any method known to those skilled in the art, including but not limited to the Preferred Technology described herein, kinase assays, enzyme assays, binding assays and other functional assays, immunoassays, and western blotting. In one embodiment, the EOPIs are separated on a 2-D gel by virtue of their MWs and pIs and visualized by staining the gel. In one embodiment, the EOPIs are stained with a fluorescent dye and imaged with a fluorescence scanner. Sypro Red (Molecular Probes, Inc., Eugene, Oreg.) is a suitable dye for this purpose. A preferred fluorescent dye is Pyridinium, 4-[2-[4-(dipentylamino)-2-trifluoromethylphenyl]ethenyl]-1-(sulfobutyl)-, inner salt. See U.S. Application No. U.S. Pat. No. 6,335,446, which is incorporated herein by reference in its entirety.

Alternatively, EOPIs can be detected in an immunoassay. In one embodiment, an immunoassay is performed by contacting a first sample from a subject to be tested with a capture reagent (e.g an antibody) under conditions such that immunospecific binding can occur if the EOPI is present, and detecting or measuring the amount of any immunospecific binding by the capture reagent. Anti-EOPI antibodies can be produced by the methods and techniques taught herein. Preferably, the anti-EOPI antibody preferentially binds to the EOPI rather than to other isoforms of the same protein. In a preferred embodiment, the anti-EOPI antibody binds to the EOPI with at least 2-fold greater affinity, more preferably at least 5-fold greater affinity, still more preferably at least 10-fold greater affinity, than to said other isoforms of the same protein.

EOPIs can be transferred from a gel to a suitable membrane (e.g. a PVDF membrane) and subsequently probed in suitable assays that include, without limitation, competitive and non-competitive assay systems using techniques such as western blots and “sandwich” immunoassays using anti-EOPI antibodies as described herein, e.g., the antibodies raised against the EOPIs of interest. The immunoblots can be used to identify those anti-EOPI antibodies displaying the selectivity required to immuno-specifically differentiate an EOPI from other isoforms encoded by the same gene.

As used herein, an “aberrant level” means a level that is increased or decreased in a first sample compared with the level in a second sample from a subject free from ErbB2 related cancer or a reference level. In one embodiment, binding of antibody in tissue sections can be used to detect aberrant EOPI localization or an aberrant level of one or more EOPIs. In a specific embodiment, antibody to an EOPI can be used to assay a first tissue sample (e.g., a breast biopsy) from a subject for the level of the EOPI where an aberrant level of EOPI is indicative of ErbB2 related cancer.

Any suitable immunoassay can be used, including, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, an EOPI can be detected in a fluid sample (e.g., blood, urine, or tissue homogenate) by means of a two-step sandwich assay. In the first step, a capture reagent (e.g., an anti-EOPI antibody) is used to capture the EOPI. The capture reagent can optionally be immobilized on a solid phase. In the second step, a directly or indirectly labelled detection reagent is used to detect the captured EOPI. In one embodiment, the detection reagent is a lectin. Any lectin can be used for this purpose that preferentially binds to the EOPI rather than to other isoforms that have the same core protein as the EOPI or to other polypeptides that share the antigenic determinant recognized by the antibody. In a preferred embodiment, the chosen lectin binds to the EOPI with at least 2-fold greater affinity, more preferably at least 5-fold greater affinity, still more preferably at least 10-fold greater affinity, than to said other isoforms that have the same core protein as the EOPI or to said other polypeptides that share the antigenic determinant recognized by the antibody. Based on the present description, a lectin that is suitable for detecting a given EOPI can readily be identified by methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174 (which is incorporated herein by reference in its entirety). Lectins with the desired oligosaccharide specificity can be identified, for example, by their ability to detect the EOPI in a 2D gel, in a replica of a 2D gel following transfer to a suitable solid substrate such as a nitrocellulose membrane, or in a two-step assay following capture by capture reagent. In an alternative embodiment, the detection reagent is an antibody, e.g., an antibody that immunospecifically detects post-translational modifications, such as an antibody that immunospecifically binds to phosphorylated amino acids. Examples of such antibodies include those that bind to phosphotyrosine (BD Transduction Laboratories, 2002, catalog nos.:P11120; P39020), those that bind to phosphoserine (Zymed Laboratories Inc. 2002, South San Francisco, Calif., catalog no. 61-8100) and those that bind to phosphothreonine (Zymed Laboratories Inc., 2002, South San Francisco, Calif., catalog nos. 71-8200, 13-9200).

If desired, a gene encoding an EOPI, a related gene, or related nucleic acid sequences or subsequences, including complementary sequences, can also be used in hybridization assays. A nucleotide encoding an EOPI, or subsequences thereof comprising at least 8 nucleotides, preferably at least 12 nucleotides, and most preferably at least 15 nucleotides can be used as a hybridization probe. Hybridization assays can be used for detection, prognosis, diagnosis, or monitoring of conditions, disorders, or disease states, associated with aberrant expression of genes encoding EOPIs, or for differential diagnosis of subjects with signs or symptoms suggestive of ErbB2 related cancer. In particular, such a hybridization assay can be carried out by a method comprising contacting a subject's sample containing nucleic acid with a nucleic acid probe capable of hybridizing to a DNA or RNA that encodes an EOPI, under conditions such that hybridization can occur, and detecting or measuring any resulting hybridization. Nucleotides can be used for therapy of subjects having ErbB2 related cancer, as described below.

The invention also provides diagnostic kits, comprising an anti-EOPI antibody. In addition, such a kit may optionally comprise one or more of the following: (1) instructions for using the anti-EOPI antibody for diagnosis, prognosis, therapeutic monitoring or any combination of these applications; (2) a labelled binding partner to the antibody; (3) a solid phase (such as a reagent strip) upon which the anti-EOPI antibody is immobilized; and (4) a label or insert indicating regulatory approval for diagnostic, prognostic or therapeutic use or any combination thereof. If no labelled binding partner to the antibody is provided, the anti-EOPI antibody itself can be labelled with a detectable marker, e.g., a chemiluminescent, enzymatic, fluorescent, or radioactive moiety.

The invention also provides a kit comprising a nucleic acid probe capable of hybridizing to RNA encoding an EOPI. In a specific embodiment, a kit comprises in one or more containers a pair of primers (e.g., each in the size range of 6-30 nucleotides, more preferably 10-30 nucleotides and still more preferably 10-20 nucleotides) that under appropriate reaction conditions can prime amplification of at least a portion of a nucleic acid encoding an EOPI, such as by polymerase chain reaction (see, e.g., Innis et al., 1990, PCR Protocols, Academic Press, Inc., San Diego, Calif.), ligase chain reaction (see EP 320,308) use of Q replicase, cyclic probe reaction, or other methods known in the art.

Kits are also provided which allow for the detection of a plurality of EOPIs or a plurality of nucleic acids each encoding an EOPI. A kit can optionally further comprise a predetermined amount of an isolated EOPI protein or a nucleic acid encoding an EOPI, e.g., for use as a standard or control.

Statistical Techniques For Identifying EOPI And EOF Clusters

The uni-variate differential analysis tools, such as fold changes, Wilcoxon rank sum test and t-test, are useful in identifying individual EOFs or EOPIs that are diagnostically associated with ErbB2 related cancer or in identifying individual EOPIs that regulate the disease process. In most cases, however, those skilled in the art appreciate that the disease process is associated with a combination of EOFs or EOPIs (and to be regulated by a combination of EOPIs), rather than individual EOFs and EOPIs in isolation. The strategies for discovering such combinations of EOFs and EOPIs differ from those for discovering individual EOFs and EOPIs. In such cases, each individual EOF and EOPIs can be regarded as one variable and the disease can be regarded as a joint, multi-variate effect caused by interaction of these variables.

The following steps can be used to identify markers from data produced by the Preferred Technology.

The first step is to identify a collection of EOFs or EOPIs that individually show a significant aberrant expression in ErbB2 related cancer. The association between the identified EOFs or EOPIs and ErbB2 related cancer need not be as highly significant as is desirable when an individual EOF or EOPI is used as a diagnostic. Any of the tests discussed above (fold changes, Wilcoxon rank sum test, etc.) can be used at this stage. Once a suitable collection of EOFs or EOPIs has been identified, a sophisticated multi-variate analysis capable of identifying clusters can then be used to estimate the significant multivariate associations with ErbB2 related cancer.

Linear Discriminant Analysis (LDA) is one such procedure, which can be used to detect significant association between a cluster of variables (i.e., EOFs or EOPIs) and ErbB2 related cancer. In performing LDA, a set of weights is associated with each variable (i.e., EOF or EOPI) so that the linear combination of weights and the measured values of the variables can identify the disease state by discriminating between subjects having ErbB2 related cancer and subjects free from ErbB2 related cancer. Enhancements to the LDA allow stepwise inclusion (or removal) of variables to optimize the discriminant power of the model. The result of the LDA is therefore a cluster of EOFs or EOPIs, which can be used without limitation for diagnosis, prognosis, therapy or drug development. Other enhanced variations of LDA, such as Flexible Discriminant Analysis permit the use of non-linear combinations of variables to discriminate a disease state from a normal state. The results of the discriminant analysis can be verified by post-hoc tests and also by repeating the analysis using alternative techniques such as classification trees.

A further category of EOFs or EOPIs can be identified by qualitative measures by comparing the percentage feature presence of an EOF or EOPI of a first sample or sample set (e.g., samples from diseased subjects) with the percentage feature presence of an EOF or EOPI in a second sample or sample set (e.g., samples from control subjects). The “percentage feature presence” of an EOF or EOPI is the percentage of samples in a sample set in which the EOF or EOPI is detectable by the detection method of choice. For example, if an EOF is detectable in 95 percent of samples from diseased subjects, the percentage feature presence of that EOF in that sample set is 95 percent. If only 5 percent of samples from non-diseased subjects have detectable levels of the same EOF, detection of that EOF in the sample of a subject would suggest that it is likely that the subject suffers from ErbB2 related cancer.

Use in Clinical Studies

The diagnostic methods and compositions of the present invention can assist in monitoring a clinical study, e.g. to evaluate drugs for therapy of ErbB2 related cancer. In one embodiment, candidate molecules are tested for their ability to restore EOF or EOPI levels in a subject having ErbB2 related cancer to levels found in subjects free from ErbB2 related cancer or, in a treated subject (e.g. a subject treated with but not limited to taxol, cyclophosphamide, taxomifen, fluorouracil or doxorubicin) to preserve EOF or EOPI levels at or near non-ErbB2 related cancer values. The levels of one or more EOFs or EOPIs can be assayed.

In another embodiment, the methods and compositions of the present invention are used to screen candidates for a clinical study to identify individuals having ErbB2 related cancer such individuals can then be either excluded from or included in the study or can be placed in a separate cohort for treatment or analysis. If desired, the candidates can concurrently be screened to identify individuals with ErbB2 related cancer; procedures for these screens are well known in the art.

Purification of EOPIs

In particular aspects, the invention provides isolated mammalian EOPIs, preferably human EOPIs, and fragments thereof which comprise an antigenic determinant (i.e., can be recognized by an antibody) or which are otherwise functionally active, as well as nucleic acid sequences encoding the foregoing. “Functionally active” as used herein refers to material displaying one or more functional activities associated with a full-length (wild-type) EOPI, e.g., binding to an EOPI substrate or EOPI binding partner, antigenicity (binding to an anti-EOPI antibody), immunogenicity, enzymatic activity and the like.

In specific embodiments, the invention provides fragments of an EOPI comprising at least 5 amino acids, at least 10 amino acids, at least 50 amino acids, or at least 75 amino acids. Fragments lacking some or all of the regions of an EOPI are also provided, as are polypeptides (e.g., fusion proteins) comprising such fragments. Nucleic acids encoding the foregoing are provided.

Once a recombinant nucleic acid which encodes the EOPI, EOPI fragment, or a precursor of the EOPI is identified, the gene product can be analyzed. This is achieved by assays based on the physical or functional properties of the product, including radioactive labeling of the product followed by analysis by gel electrophoresis, immunoassay, etc.

The EOPIs identified herein can be isolated and purified by standard methods including chromatography (e.g., ion exchange, affinity, and sizing column chromatography), centrifugation, differential solubility, or by any other standard technique for the purification of proteins.

Alternatively, once a recombinant nucleic acid that encodes the EOPI is identified, the entire amino acid sequence of the EOPI can be deduced from the nucleotide sequence of the gene coding region contained in the recombinant nucleic acid. As a result, the protein can be synthesized by standard chemical methods known in the art (e.g., see Hunkapiller et al., 1984, Nature 310:105-111).

In another alternative embodiment, native EOPIs can be purified from natural sources, by standard methods such as those described above (e.g., immunoaffinity purification).

In a preferred embodiment, EOPIs are isolated by the Preferred Technology described supra. For preparative-scale runs, a narrow-range “zoom gel” having a pH range of 2 pH units or less is preferred for the isoelectric step, according to the method described in Westermeier, 1993, Electrophoresis in Practice (VCH, Weinheim, Germany), pp. 197-209 (which is incorporated herein by reference in its entirety); this modification permits a larger quantity of a target protein to be loaded onto the gel, and thereby increases the quantity of isolated EOPIs that can be recovered from the gel. When used in this way for preparative-scale runs, the Preferred Technology typically provides up to 100 ng, and can provide up to 1000 ng, of an isolated EOPI in a single run. Those of skill in the art will appreciate that a zoom gel can be used in any separation strategy which employs gel isoelectric focusing.

The invention thus provides an EOPI, EOPI fragment, EOPI-related polypeptide or the EOPI-fusion protein; any of the foregoing can be produced by recombinant DNA techniques or by chemical synthetic methods.

Isolation of DNA Encoding an EOPI

Specific embodiments for the cloning of a gene encoding an EOPI are presented below by way of example and not of limitation.

The nucleotide sequences of the present invention, including DNA and RNA, and comprising a sequence encoding an EOPI, EOPI fragment, EOPI-related polypeptide or the EOPI-fusion protein, may be synthesized using methods known in the art, such as using conventional chemical approaches or polymerase chain reaction (PCR) amplification. The nucleotide sequences of the present invention also permit the identification and cloning of the gene encoding an EOPI homolog or EOPI ortholog including, for example, by screening cDNA libraries, genomic libraries or expression libraries.

For example, to clone a gene encoding an EOPI by PCR techniques, anchored degenerate oligonucleotides (or a set of most likely oligonucleotides) can be designed for all EOPI peptide fragments identified as part of the same protein. PCR reactions under a variety of conditions can be performed with relevant cDNA and genomic DNAs (e.g., from tissue or body fluid or from cells of the immune system) from one or more species. Also vectorette reactions can be performed on any available cDNA and genomic DNA using the oligonucleotides (which preferably are nested) as above. Vectorette PCR is a method that enables the amplification of specific DNA fragments in situations where the sequence of only one primer is known. Thus, it extends the application of PCR to stretches of DNA where the sequence information is only available at one end. (Arnold C, 1991, PCR Methods Appl. 1(1):3942; Dyer KD, Biotechniques, 1995, 19(4):550-2). Vectorette PCR may be performed with probes that are, for example, anchored degenerate oligonucleotides (or most likely oligonucleotides) coding for EOPI peptide fragments, using as a template a genomic library or cDNA library pools.

Anchored degenerate oligonucleotides (and most likely oligonucleotides) can be designed for all EOPI peptide fragments. These oligonucleotides may be labelled and hybridized to filters containing cDNA and genomic DNA libraries. Oligonucleotides to different peptides from the same protein will often identify the same members of the library. The cDNA and genomic DNA libraries may be obtained from any suitable or desired mammalian species, for example from humans.

Nucleotide sequences comprising a nucleotide sequence encoding EOPI, EOPI fragment, EOPI-related polypeptide or the EOPI-fusion protein of the present invention are useful for their ability to hybridize selectively with complementary stretches of genes encoding other proteins. Depending on the application, a variety of hybridization conditions may be employed to obtain nucleotide sequences at least 30%, 35%, 40%, 45%, 50%, 55%, 60%, 65%, 70%, 75%, 80%, 85%, 90%, 95% or 99% identical, or 100% identical, to the sequence of a nucleotide encoding an EOPI.

For a high degree of selectivity, relatively stringent conditions are used to form the duplexes, such as low salt or high temperature conditions. Hybridization conditions can also be rendered more stringent by the addition of increasing amounts of formamide, to destabilize the hybrid duplex. Thus, particular hybridization conditions can be readily manipulated, and will generally be chosen depending on the desired results. In general, convenient hybridization temperatures in the presence of 50% formamide are: 42° C. for a probe which is 95 to 100% identical to the fragment of a gene encoding an EOPI, 37° C. for 90 to 95% identity and 32° C. for 70 to 90% identity.

In the preparation of genomic libraries, DNA fragments are generated, some of which will encode parts or the whole of an EOPI. Any suitable method for preparing DNA fragments may be used in the present invention. For example, the DNA may be cleaved at specific sites using various restriction enzymes. Alternatively, one may use DNAse in the presence of manganese to fragment the DNA, or the DNA can be physically sheared, as for example, by sonication. The DNA fragments can then be separated according to size by standard techniques, including but not limited to agarose and polyacrylamide gel electrophoresis, column chromatography and sucrose gradient centrifugation. The DNA fragments can then be inserted into suitable vectors, including but not limited to plasmids, cosmids, bacteriophages lambda or T4, and yeast artificial chromosome (YAC). (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II; Ausubel F. M. et al., eds., 1989, Current Protocols in Molecular Biology, Vol. 1, Green Publishing Associates, Inc., and John Wiley & sons, Inc., New York). The genomic library may be screened by nucleic acid hybridization to labelled probe (Benton and Davis, 1977, Science 196:180; Grunstein and Hogness, 1975, Proc. Natl. Acad. Sci. U.S.A. 72:3961).

Based on the present description, the genomic libraries may be screened with labelled degenerate oligonucleotide probes corresponding to the amino acid sequence of any peptide of the EOPI using optimal approaches well known in the art. Any probe used is at least 10 nucleotides, at least 15 nucleotides, at least 20 nucleotides, at least 25 nucleotides, at least 30 nucleotides, at least 40 nucleotides, at least 50 nucleotides, at least 60 nucleotides, at least 70 nucleotides, at least 80 nucleotides, or at least 100 nucleotides in length.

In Tables VI, VII, VIII and IX above, some EOPIs disclosed herein were found to correspond to isoforms of previously identified proteins encoded by genes whose sequences are publicly known. (Sequence analysis and protein identification of EOPIs was carried out using the methods described in Section 6.1.14). To screen such a gene, any probe may be used that is complementary to the gene or its complement; preferably the probe is 10 nucleotides or longer, more preferably 15 nucleotides or longer. The SWISS-PROT and trEMBL databases (held by the Swiss Institute of Bioinformatics (SIB) and the European Bioinformatics Institue (EBI) which are available at http://www.expasy.com/) and the GenBank database (held by the National Institute of Health (NIH) which is available at http://www.ncbi.nlm.nih.gov/GenBank/) provide protein sequences for the EOPIs, listed in Table m, under the following accession numbers (Table IV) and each sequence is incorporated herein by reference. Preferred EOPIs of the invention are EOF-86, EOF-106, EOF-163, EOF-183, EOF-201, EOF-396, EOF-483, EOF-630, EOF-634, EOF-683 and EOF-693.

TABLE IV
Sequences encoding EOPIs, EOPI Related Proteins

| Feature | Isoform | Accession |
|---|---|---|
| (EOF) | (EOPI) | Number |
| EOF-86 | EOPI-19 | O43846 |
| EOF-106 | EOPI-22 | AF046025.2 |
| EOF-163 | EOPI-92 | O76070 |
| EOF-183 | EOPI-95 | P07900 |
| EOF-201 | EOPI-34 | Q15019 |
| EOF-396 | EOPI-115 | P42224 |
| EOF-483 | EOPI-125 | P22314 |
| EOF-630 | EOPI-59 | Q04760 |
| EOF-634 | EOPI-60 | P47813 |
| EOF-683 | EOPI-62 | O14737 |
| EOF-693 | EOPI-63 | P06703 |
| EOF-34 | EOPI-67 | O00469 |
| EOF-35 | EOPI-1 | O00469 |
| EOF-44 | EOPI-2 | P33993 |
| EOF-48 | EOPI-3 | P11021 |
| EOF-51 | EOPI-4 | Q12931 |
| EOF-57 | EOPI-70 | P13796 |
| EOF-61 | EOPI-71 | 10435998 |
| EOF-63 | EOPI-5 | P08670 |
| EOF-66 | EOPI-72 | AK025881.1 |
| EOF-67 | EOPI-6 | P47895 |
| EOF-70 | EOPI-7 | P04350 |
| EOF-70 | EOPI-8 | P13646 |
| EOF-72 | EOPI-9 | P05787 |
| EOF-73 | EOPI-11 | P07437 |
| EOF-74 | EOPI-12 | Q16401 |
| EOF-75 | EOPI-13 | O46546 |
| EOF-76 | EOPI-14 | P07437 |
| EOF-76 | EOPI-15 | P13646 |
| EOF-80 | EOPI-16 | P19012 |
| EOF-81 | EOPI-17 | P02571 |
| EOF-81 | EOPI-18 | P10644 |
| EOF-83 | EOPI-73 | P17980 |
| EOF-84 | EOPI-74 | X56134.1 |
| EOF-87 | EOPI-75 | O43852 |
| EOF-90 | EOPI-20 | P05783 |
| EOF-94 | EOPI-77 | P23526 |
| EOF-94 | EOPI-78 | O75821 |
| EOF-99 | EOPI-80 | 8895085 |
| EOF-102 | EOPI-21 | AF282596 |
| EOF-104 | EOPI-81 | P16930 |
| EOF-107 | EOPI-23 | P36952 |
| EOF-110 | EOPI-24 | P51570 |
| EOF-111 | EOPI-82 | Q01105 |
| EOF-120 | EOPI-84 | 7023317 |
| EOF-129 | EOPI-85 | P42655 |
| EOF-133 | EOPI-86 | P21266 |
| EOF-135 | EOPI-87 | Q29459 |
| EOF-136 | EOPI-25 | P25788 |
| EOF-137 | EOPI-26 | P40261 |
| EOF-138 | EOPI-88 | O95833 |
| EOF-139 | EOPI-89 | P25788 |
| EOF-145 | EOPI-90 | P52566 |
| EOF-149 | EOPI-27 | P09211 |
| EOF-153 | EOPI-91 | P07741 |
| EOF-156 | EOPI-28 | AF136630 |
| EOF-166 | EOPI-29 | P09382 |
| EOF-167 | EOPI-94 | P35080 |
| EOF-182 | EOPI-30 | P08238 |
| EOF-188 | EOPI-96 | 7212807 |
| EOF-196 | EOPI-31 | P46055 |
| EOF-196 | EOPI-32 | Q13838 |
| EOF-197 | EOPI-33 | P48637 |
| EOF-206 | EOPI-97 | P08758 |
| EOF-225 | EOPI-98 | P09382 |
| EOF-242 | EOPI-35 | P11142 |
| EOF-251 | EOPI-99 | 1490513 |
| EOF-254 | EOPI-100 | P78417 |
| EOF-263 | EOPI-102 | P08238 |
| EOF-267 | EOPI-104 | P21281 |
| EOF-342 | EOPI-105 | 6563246 |
| EOF-349 | EOPI-106 | O00469 |
| EOF-350 | EOPI-107 | O00469 |
| EOF-352 | EOPI-108 | P42224 |
| EOF-361 | EOPI-36 | P05787 |
| EOF-366 | EOPI-110 | 901998 |
| EOF-370 | EOPI-37 | P06748 |
| EOF-371 | EOPI-111 | P52907 |
| EOF-371 | EOPI-112 | P11177 |
| EOF-372 | EOPI-38 | P07195 |
| EOF-376 | EOPI-39 | P09936 |
| EOF-383 | EOPI-113 | P37802 |
| EOF-387 | EOPI-40 | P16475 |
| EOF-389 | EOPI-114 | P09382 |
| EOF-400 | EOPI-116 | P13796 |
| EOF-415 | EOPI-117 | 6014636 |
| EOF-419 | EOPI-118 | P24480 |
| EOF-433 | EOPI-119 | Q05682 |
| EOF-435 | EOPI-121 | P46459 |
| EOF-435 | EOPI-120 | Q29443 |
| EOF-444 | EOPI-122 | 901998 |
| EOF-460 | EOPI-123 | 551606 |
| EOF-482 | EOPI-124 | P18206 |
| EOF-489 | EOPI-126 | Q10567 |
| EOF-490 | EOPI-41 | P21359 |
| EOF-493 | EOPI-42 | P08238 |
| EOF-501 | EOPI-127 | P08107 |
| EOF-510 | EOPI-43 | P49368 |
| EOF-513 | EOPI-129 | 2529707 |
| EOF-523 | EOPI-44 | AAD33873 |
| EOF-526 | EOPI-45 | AK001711 |
| EOF-527 | EOPI-46 | O76096 |
| EOF-532 | EOPI-130 | P26641 |
| EOF-533 | EOPI-47 | P49411 |
| EOF-534 | EOPI-48 | O46546 |
| EOF-536 | EOPI-131 | J03503.1 |
| EOF-544 | EOPI-49 | P09494 |
| EOF-546 | EOPI-50 | P09494 |
| EOF-550 | EOPI-133 | 4929635 |
| EOF-552 | EOPI-51 | P47756 |
| EOF-553 | EOPI-134 | P30040 |
| EOF-554 | EOPI-136 | P35232 |
| EOF-556 | EOPI-137 | P28070 |
| EOF-560 | EOPI-139 | P17080 |
| EOF-562 | EOPI-140 | P09211 |
| EOF-574 | EOPI-52 | P19105 |
| EOF-578 | EOPI-53 | X79302.1 |
| EOF-582 | EOPI-54 | P05387 |
| EOF-582 | EOPI-55 | P16475 |
| EOF-584 | EOPI-141 | P29762 |
| EOF-609 | EOPI-56 | P55010 |
| EOF-611 | EOPI-57 | Q16664 |
| EOF-612 | EOPI-58 | Q04695 |
| EOF-636 | EOPI-143 | P24928 |
| EOF-665 | EOPI-61 | P05388 |
| EOF-711 | EOPI-144 | P25788 |
| EOF-713 | EOPI-64 | P21964 |
| EOF-721 | EOPI-65 | P35080 |
| EOF-724 | EOPI-145 | 1490513 |

For any EOPI, degenerate probes, or probes taken from the sequences described above by accession number may be used for screening. In the case of degenerate probes, they can be constructed from the partial amino sequence information obtained from tandem mass spectra of tryptic digest peptides of the EOPI. To screen such a gene, any probe may be used that is complementary to the gene or its complement; the probe is 10 nucleotides or longer, preferably 15 nucleotides or longer. When a library is screened, clones with insert DNA encoding the EOPI or a fragment thereof will hybridize to one or more members of the corresponding set of degenerate oligonucleotide probes (or their complement). Hybridization of such oligonucleotide probes to genomic libraries is carried out using methods known in the art. For example, hybridization with one of the above-mentioned degenerate sets of oligonucleotide probes, or their complement (or with any member of such a set, or its complement) can be performed under highly stringent or moderately stringent conditions as defined supra, or can be carried out in 2× SSC, 1.0% SDS at 50° C. and washed using the washing conditions described supra for highly stringent or moderately stringent hybridization.

In yet another aspect of the invention, clones containing nucleotide sequences encoding the EOPI, EOPI fragment, EOPI-related polypeptide or the EOPI-fusion protein any of the foregoing may also be obtained by screening expression libraries. For example, DNA from the relevant source is isolated and random fragments are prepared and ligated into an expression vector (e.g., a bacteriophage, plasmid, phagemid or cosmid) such that the inserted sequence in the vector is capable of being expressed by the host cell into which the vector is then introduced. Various screening assays can then be used to select for the expressed EOPI, EOPI fragment, EOPI-related polypeptide or the EOPI-fusion protein. In one embodiment, the various anti-EOPI antibodies of the invention can be used to identify the desired clones using methods known in the art. See, for example, Harlow and Lane, 1988, Antibodies: A Laboratory Manual, Cold Spring Harbor Laboratory Press, Cold Spring Harbor, N.Y., Appendix IV. Colonies or plaques from the library are brought into contact with the antibodies to identify those clones that bind antibody.

In an embodiment, colonies or plaques containing DNA that encodes EOPI, EOPI fragment, EOPI-related polypeptide or the EOPI-fusion protein can be detected using DYNA Beads according to Olsvick et al., 29th ICAAC, Houston, Tex. 1989, incorporated herein by reference. Anti-EOPI antibodies are crosslinked to tosylated DYNA Beads M280, and these antibody-containing beads are then contacted with colonies or plaques expressing recombinant polypeptides. Colonies or plaques expressing an EOPI, EOPI fragment, EOPI-related polypeptide or the EOPI-fusion protein are identified as any of those that bind the beads.

Alternatively, the anti-EOPI antibodies can be nonspecifically immobilized to a suitable support, such as silica or Celite® resin. This material is then used to adsorb to bacterial colonies expressing the EOPI, EOPI fragment, EOPI-related polypeptide or the EOPI-fusion protein as described herein.

In another aspect, PCR amplification may be used to isolate from genomic DNA a substantially pure DNA (i.e., a DNA substantially free of contaminating nucleic acids) encoding the entire EOPI or a part thereof. Preferably such a DNA is at least 95% pure, more preferably at least 99% pure. Oligonucleotide sequences, degenerate or otherwise, that correspond to peptide sequences of EOPIs disclosed herein can be used as primers.

PCR can be carried out, e.g., by use of a Perkin-Elmer Cetus thermal cycler and Taq polymerase (Gene Amp™ or AmpliTaq™ DNA polymerase). One can choose to synthesize several different degenerate primers, for use in the PCR reactions. It is also possible to vary the stringency of hybridization conditions used in priming the PCR reactions, to allow for greater or lesser degrees of nucleotide sequence similarity between the degenerate primers and the corresponding sequences in the DNA. After successful amplification of a segment of the sequence encoding an EOPI, that segment may be molecularly cloned and sequenced, and utilized as a probe to isolate a complete genomic clone. This, in turn, will permit the determination of the gene's complete nucleotide sequence, the analysis of its expression, and the production of its protein product for functional analysis, as described infra.

The gene encoding an EOPI can also be identified by mRNA selection by nucleic acid hybridization followed by in vitro translation. In this procedure, fragments are used to isolate complementary mRNAs by hybridization. Such DNA fragments may represent available, purified DNA encoding an EOPI of another species (e.g., mouse, human). Immunoprecipitation analysis or functional assays (e.g., aggregation ability in vitro; binding to receptor) of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilized antibodies that specifically recognize an EOPI. A radiolabelled cDNA encoding an EOPI can be synthesized using the selected mRNA (from the adsorbed polysomes) as a template. The radiolabelled mRNA or cDNA may then be used as a probe to identify the DNA fragments encoding an EOPI from among other genomic DNA fragments.

Alternatives to isolating genomic DNA encoding an EOPI include, but are not limited to, chemically synthesizing the gene sequence itself from a known sequence or making cDNA to the mRNA which encodes the EOPI. For example, RNA for cDNA cloning of the gene encoding an EOPI can be isolated from cells which express the EOPI. Those skilled in the art will understand from the present description that other methods may be used and are within the scope of the invention.

Any suitable eukaryotic cell can serve as the nucleic acid source for the molecular cloning of the gene encoding an EOPI. The nucleic acid sequences encoding the EOPI can be isolated from vertebrate, mammalian, primate, human, porcine, bovine, feline, avian, equine, canine or murine sources. The DNA may be obtained by standard procedures known in the art from cloned DNA (e.g., a DNA “library”), by chemical synthesis, by cDNA cloning, or by the cloning of genomic DNA, or fragments thereof, purified from the desired cell. (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. 1, II.) Clones derived from genomic DNA may contain regulatory and intron DNA regions in addition to coding regions; clones derived from cDNA will contain only exon sequences.

The identified and isolated gene or cDNA can then be inserted into any suitable cloning vector. A large number of vector-host systems known in the art may be used. As those skilled in the art will appreciate, the only limitation is that the vector system chosen be compatible with the host cell used. Such vectors include, but are not limited to, bacteriophages such as lambda derivatives, plasmids such as PBR322 or pUC plasmid derivatives or the Bluescript™ vector (Stratagene) or modified viruses such as adenoviruses, adeno-associated viruses or retroviruses. The insertion into a cloning vector can be accomplished, for example, by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and the gene encoding an EOPI may be modified by homopolymeric tailing. Recombinant molecules can be introduced into host cells via transformation, transfection, infection, electroporation, etc., so that many copies of the gene sequence are generated.

In specific embodiments, transformation of host cells with recombinant DNA molecules that incorporate the isolated gene encoding the EOPI, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The nucleotide sequences of the present invention include nucleotide sequences encoding amino acid sequences with substantially the same amino acid sequences as native EOPI, nucleotide sequences encoding amino acid sequences with functionally equivalent amino acids, nucleotide sequences encoding EOPI, EOPI fragment, EOPI-related polypeptide or the EOPI-fusion protein.

In a specific embodiment, an isolated nucleic acid molecule encoding an EOPI-related polypeptide can be created by introducing one or more nucleotide substitutions, additions or deletions into the nucleotide sequence of an EOPI such that one or more amino acid substitutions, additions or deletions are introduced into the encoded protein. Standard techniques known to those of skill in the art can be used to introduce mutations, including, for example, site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

Expression of DNA Encoding EOPIs

The nucleotide sequence coding for an EOPI, EOPI fragment or EOPI-related polypeptide or other derivative of any of the foregoing, can be inserted into an appropriate expression vector, i.e., a vector which contains the necessary elements for the transcription and translation of the inserted protein-coding sequence. The necessary transcriptional and translational signals can also be supplied by the native gene encoding the EOPI or its flanking regions, or the native gene encoding the EOPI-related polypeptide or its flanking regions. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In specific embodiments, a nucleotide sequence encoding a human gene (or a nucleotide sequence encoding a functionally active portion of a human EOPI) is expressed. In yet another embodiment, a fragment of an EOPI comprising a domain of the EOPI is expressed.

Any of the methods previously described for the insertion of DNA fragments into a vector may be used to construct expression vectors containing a chimeric gene consisting of appropriate transcriptional and translational control signals and the protein coding sequences. These methods may include in vitro recombinant DNA and synthetic techniques and in vivo recombinants (genetic recombination). Expression of nucleic acid sequence encoding an EOPI or fragment thereof may be regulated by a second nucleic acid sequence so that the EOPI or fragment is expressed in a host transformed with the recombinant DNA molecule. For example, expression of an EOPI may be controlled by any promoter or enhancer element known in the art. Promoters which may be used to control the expression of the gene encoding an EOPI or an EOPI-related polypeptide include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:3942), the tetracycline (Tet) promoter (Gossen et al., 1995, Proc. Nat. Acad. Sci. USA 89:5547-5551); prokaryotic expression vectors such as the b-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus 35S RNA promoter (Gardner, et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krunmlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Com. 253:818-823); glial fibrillary acidic protein (GFAP) promoter which is active in astrocytes (Gomes et al., 1999, Braz J Med Biol Res 32(5):619-631; Morelli et al., 1999, Gen. Virol. 80:571-83) and gonadotropic releasing hormone gene control region which is active in the hypothalamus (Mason et al., 1986, Science 234:1372-1378).

In a specific embodiment, a vector is used that comprises a promoter operably linked to an EOPI-encoding nucleic acid, one or more origins of replication, and, optionally, one or more selectable markers (e.g., an antibiotic resistance gene).

In a specific embodiment, an expression construct is made by subcloning an EOPI, EOPI fragment or EOPI-related polypeptide coding sequence into the EcoRI restriction site of each of the three pGEX vectors (Glutathione S-Transferase expression vectors; Smith and Johnson, 1988, Gene 7:3140). This allows for the expression of the EOPI product or EOPI-related polypeptide from the subclone in the correct reading frame.

In mammalian host cells, a number of viral-based expression systems may be utilized. In cases where an adenovirus is used as an expression vector, the EOPI coding sequence or EOPI-related polypeptide coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing inserts of a gene encoding an EOPI, EOPI fragment or EOPI-related polypeptide can be identified by three general approaches: (a) nucleic acid hybridization, (b) presence or absence of “marker” gene functions, and (c) expression of inserted sequences. In the first approach, the presence of a gene encoding an EOPI inserted in an expression vector can be detected by nucleic acid hybridization using probes comprising sequences that are homologous to an inserted gene encoding an EOPI. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a gene encoding an EOPI in the vector. For example, if the gene encoding the EOPI is inserted within the marker gene sequence of the vector, recombinants containing the gene encoding the EOPI insert can be identified by the absence of the marker gene function. In the third approach, recombinant expression vectors can be identified by assaying the gene product (i.e., EOPI) expressed by the recombinant. Such assays can be based, for example, on the physical or functional properties of the EOPI in in vitro assay systems, e.g., binding with anti-EOPI antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered EOPI or EOPI-related polypeptide may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, HEK293, 3T3, W138, and in particular, neuronal cell lines such as, for example, SK-N-AS, SK-N-FI, SK-N-DZ human neuroblastomas (Sugimoto et al., 1984, J. Natl. Cancer Inst. 73: 51-57), SK-N-SH human neuroblastoma (Biochim. Biophys. Acta, 1982, 704: 450-460), Daoy human cerebellar medulloblastoma (He et al., 1992, Cancer Res. 52: 1144-1148) DBTRG-05MG glioblastoma cells (Kruse et al., 1992, In Vitro Cell. Dev. Biol. 28A: 609-614), IMR-32 human neuroblastoma (Cancer Res., 1970, 30: 2110-2118), 1321N1 human astrocytoma (Proc. Natl. Acad. Sci. USA,1977, 74: 4816), MOG-G-CCM human astrocytoma (Br. J. Cancer, 1984, 49: 269), U87MG human glioblastoma-astrocytoma (Acta Pathol. Microbiol. Scand., 1968, 74: 465-486), A172 human glioblastoma (Olopade et al., 1992, Cancer Res. 52: 2523-2529), C6 rat glioma cells (Benda et al., 1968, Science 161: 370-371), Neuro-2a mouse neuroblastoma (Proc. Natl. Acad. Sci. USA, 1970, 65: 129-136), NB41A3 mouse neuroblastoma (Proc. Natl. Acad. Sci. USA, 1962, 48: 1184-1190), SCP sheep choroid plexus (Bolin et al., 1994, J. Virol. Methods 48: 211-221), G355-5, PG-4 Cat normal astrocyte (Haapala et al., 1985, J. Virol. 53: 827-833), Mpf ferret brain (Trowbridge et al., 1982, In Vitro 18: 952-960), and normal cell lines such as, for example, CTX TNA2 rat normal cortex brain (Radany et al., 1992, Proc. Natl. Acad. Sci. USA 89: 6467-6471) such as, for example, CRL7030 and Hs578Bst. Furthermore, different vector/host expression systems may effect processing reactions to different extents.

For long-term, high-yield production of recombinant proteins, stable expression is preferred. For example, cell lines which stably express the EOPI may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the EOPI. Such engineered cell lines may be particularly useful in screening and evaluation of agents that affect the endogenous activity of the EOPI.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt-cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

In other specific embodiments, the EOPI, fragment, analog, or derivative may be expressed as a fusion, or chimeric protein product (comprising the protein, fragment, analog, or derivative joined via a peptide bond to a heterologous protein sequence). For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification, increase half-life in vivo, and enhance the delivery of an antigen across an epithelial barrier to the immune system. An increase in the half-life in vivo and facilitated purification has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., WO 96/22024 and WO 99/04813).

Nucleic acids encoding an EOPI, a fragment of an EOPI, an EOPI-related polypeptide, or a fragment of an EOPI-related polypeptide can fused to an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897).

Fusion proteins can be made by ligating the appropriate nucleic acid sequences encoding the desired amino acid sequences to each other by methods known in the art, in the proper coding frame, and expressing the chimeric product by methods commonly known in the art. Alternatively, a fusion protein may be made by protein synthetic techniques, e.g., by use of a peptide synthesizer.

Both cDNA and genomic sequences can be cloned and expressed.

Domain Structure of EOPIs

Domains of some EOPIs are known in the art and have been described in the scientific literature. Moreover, domains of an EOPI can be identified using techniques known to those of skill in the art. For example, one or more domains of an EOPI can be identified by using one or more of the following programs: ProDom, TMpred, and SAPS. ProDom compares the amino acid sequence of a polypeptide to a database of compiled domains (see, e.g., http://www.toulouse.inra.fr/prodom.html; Corpet F., Gouzy J. & Kahn D., 1999, Nucleic Acids Res., 27:263-267). TMpred predicts membrane-spanning regions of a polypeptide and their orientation. This program uses an algorithm that is based on the statistical analysis of TMbase, a database of naturally occuring transmembrane proteins (see, e.g., http://www.ch.embnet.org/software/TMPRED_form.html; Hofmann & Stoffel. (1993) “TMbase—A database of membrane spanning proteins segments.” Biol. Chem. Hoppe-Seyler 347,166). The SAPS program analyzes polypeptides for statistically significant features like charge-clusters, repeats, hydrophobic regions, compositional domains (see, e.g., Brendel et al., 1992, Proc. Natl. Acad. Sci. USA 89: 2002-2006). Thus, based on the present description, the skilled artisan can identify domains of an EOPI having enzymatic or binding activity, and further can identify nucleotide sequences encoding such domains. These nucleotide sequences can then be used for recombinant expression of an EOPI fragment that retains the enzymatic or binding activity of the EOPI.

Based on the present description, the skilled artisan can identify domains of an EOPI having enzymatic or binding activity, and further can identify nucleotide sequences encoding such domains. These nucleotide sequences can then be used for recombinant expression of EOPI fragments that retain the enzymatic or binding activity of the EOPI.

In one embodiment, an EOPI has an amino acid sequence sufficiently similar to an identified domain of a known polypeptide. As used herein, the term “sufficiently similar” refers to a first amino acid or nucleotide sequence which contains a sufficient number of identical or equivalent (e.g., with a similar side chain) amino acid residues or nucleotides to a second amino acid or nucleotide sequence such that the first and second amino acid or nucleotide sequences have or encode a common structural domain or common functional activity or both.

An EOPI domain can be assessed for its function using techniques well known to those of skill in the art. For example, a domain can be assessed for its kinase activity or for its ability to bind to DNA using techniques known to the skilled artisan. Kinase activity can be assessed, for example, by measuring the ability of a polypeptide to phosphorylate a substrate. DNA binding activity can be assessed, for example, by measuring the ability of a polypeptide to bind to a DNA binding element in a electromobility shift assay.

Production of Antibodies to EOPIs

According to the invention an EOPI, EOPI fragment, EOPI-related polypeptide or the EOPI-fusion protein or derivative of any of the foregoing may be used as an immunogen to generate antibodies which immunospecifically bind such an immunogen. Such immunogens can be isolated by any convenient means, including the methods described above. Antibodies of the invention include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

In one embodiment, antibodies that recognize gene products of genes encoding EOPIs are publicly available. For example, antibodies that recognize these EOPI, EOPI fragment, EOPI-related polypeptide or the EOPI-fusion proteins include antibodies that can be purchased from commercial sources. In another embodiment, methods known to those skilled in the art are used to produce antibodies that recognize an EOPI, EOPI fragment, EOPI-related polypeptide or EOPI-fusion protein or derivatives of any of the foregoing.

In one embodiment of the invention, antibodies to a specific domain of an EOPI are produced. In a specific embodiment, hydrophilic fragments of an EOPI are used as immunogens for antibody production.

In the production of antibodies, screening for the desired antibody can be accomplished by techniques known in the art, e.g. ELISA (enzyme-linked immunosorbent assay). For example, to select antibodies, which recognize a specific domain of an EOPI, one may assay generated hybridomas for a product which binds to an EOPI fragment containing such domain. For selection of an antibody that specifically binds a first EOPI homolog but which does not specifically bind to (or binds less avidly to) a second EOPI homolog, one can select on the basis of positive binding to the first EOPI homolog and a lack of binding to (or reduced binding to) the second EOPI homolog. Similarly, for selection of an antibody that specifically binds an EOPI but which does not specifically bind to (or binds less avidly to) a different isoform of the same protein (such as a different glycoform having the same core peptide as the EOPI), one can select on the basis of positive binding to the EOPI and a lack of binding to (or reduced binding to) the different isoform (e.g., a different glycoform). Thus, the present invention provides an antibody (preferably a monoclonal antibody) that binds with greater affinity (preferably at least 2-fold, more preferably at least 5-fold still more preferably at least 10-fold greater affinity) to an EOPI than to a different isoform or isoforms (e.g., glycoforms) of the EOPI.

Polyclonal antibodies, which may be used in the methods of the invention, are heterogeneous populations of antibody molecules derived from the sera of immunized animals. Unfractionated immune serum can also be used. Various procedures known in the art may be used for the production of polyclonal antibodies to an EOPI, EOPI fragment, EOPI-related polypeptide or the EOPI-fusion protein. In a particular embodiment, rabbit polyclonal antibodies to an epitope of an EOPI, EOPI fragment, EOPI-related polypeptide or the EOPI-fusion protein can be obtained. For example, for the production of polyclonal or monoclonal antibodies, various host animals can be immunized by injection with the native or a synthetic (e.g., recombinant) version of an EOPI, EOPI fragment, EOPI-related polypeptide or the EOPI-fusion protein, including but not limited to rabbits, mice, rats, etc. The Preferred Technology described herein provides isolated EOPIs suitable for such immunization. If the EOPI is purified by gel electrophoresis, the EOPI can be used for immunization with or without prior extraction from the polyacrylamide gel. Various adjuvants may be used to enhance the immunological response, depending on the host species, including, but not limited to, complete or incomplete Freund's adjuvant, a mineral gel such as aluminum hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or corynebacterium parvum. Additional adjuvants are also well known in the art.

For preparation of monoclonal antibodies (mAbs) directed toward an EOPI, EOPI fragment, EOPI-related polypeptide or the EOPI-fusion protein, any technique which provides for the production of antibody molecules by continuous cell lines in culture may be used. For example, the hybridoma technique originally developed by Kohler and Milstein (1975, Nature 256:495497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of the invention may be cultivated in vitro or in vivo. In an additional embodiment of the invention, mAbs can be produced in germ-free animals utilizing known technology (PCT/US90/02545).

The mAbs include but are not limited to human mAbs and chimeric mAbs (e.g., human-mouse chimeras). A chimeric antibody is a molecule in which different portions are derived from different animal species, such as those having a human immunoglobulin constant region and a variable region derived from a murine mAb. (See, e.g., U.S. Pat. No. 4,816,567 and U.S. Pat. No. 4,816,397.) Humanized antibodies are antibody molecules from non-human species having one or more complementarily determining regions (CDRs) from the non-human species and a framework region from a human immunoglobulin molecule. (See, e.g U.S. Pat. No. 5,585,089, which is incorporated herein by reference in its entirety.)

Chimeric and humanized mAbs can be produced by recombinant DNA techniques known in the art, for example using methods described in WO 87/02671; EP 184,187A; EP 171,496A; EP 173,494A; WO 86/01533; U.S. Pat. No. 4,816,567; EP 125,023A; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al. (1988) Science 239:1534; and Beidler et al., 1988, J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of an EOPI of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

The antibodies of the present invention can also be generated using various phage display methods known in the art. In phage display methods, functional antibody domains are displayed on the surface of phage particles which carry the polynucleotide sequences encoding them. In particular, such phage can be utilized to display antigen binding domains expressed from a repertoire or combinatorial antibody library (e.g., human or murine). Phage expressing an antigen binding domain that binds the antigen of interest can be selected or identified with antigen, e.g., using labelled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 1879-18 (1997); Burton et al., Advances in Immunology 57:191-280 (1994); PCT/GB91/01134; WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in WO 92/22324; Mullinax et al., BioTechniques 12(6):864-869 (1992); and Sawai et al., AJRI 34:26-34 (1995); and Better et al., Science 240:1041-1043 (1988).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

The invention further provides for the use of bispecific antibodies, which can be made by methods known in the art. Traditional production of full length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., 1991, EMBO J. 10:3655-3659.

According to a different and preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details for generating bispecific antibodies see, for example, Suresh et al., Methods in Enzymology, 1986, 121:210.

The invention provides functionally active fragments, derivatives or analogs of the anti-EOPI immunoglobulin molecules. Functionally active means that the fragment, derivative or analog is able to elicit anti-anti-idiotype antibodies (i.e., tertiary antibodies) that recognize the same antigen that is recognized by the antibody from which the fragment, derivative or analog is derived. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

The present invention provides antibody fragments such as, but not limited to, F(ab′)₂ fragments and Fab fragments. Antibody fragments which recognize specific epitopes may be generated by known techniques. F(ab′)₂ fragments consist of the variable region, the light chain constant region and the CH1 domain of the heavy chain and are generated by pepsin digestion of the antibody molecule. Fab fragments are generated by reducing the disulfide bridges of the F(ab′)₂ fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., 1988, Science 242:1038-1041).

In other embodiments, the invention provides fusion proteins of the immunoglobulins of the invention (or functionally active fragments thereof), for example in which the immunoglobulin is fused via a covalent bond (e.g., a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are either modified, i.e, by the covalent attachment of any type of molecule as long as such covalent attachment that does not impair immunospecific binding. For example, but not by way of limitation, the derivatives and analogs of the immunoglobulins include those that have been further modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analog or derivative may contain one or more non-classical amino acids.

The foregoing antibodies can be used in methods known in the art relating to the localization and activity of the EOPIs of the invention, e.g., for imaging these polypeptides, measuring levels thereof in appropriate physiological samples, in diagnostic methods, etc.

Expression of Antibodies

The antibodies of the invention can be produced by any method known in the art for the synthesis of antibodies, in particular, by chemical synthesis or by recombinant expression, and are preferably produced by recombinant expression techniques.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridizable to the 3′ and 5′ ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, more preferably, by generating mAbs. Alternatively, a clone encoding at least the Fab portion of the antibody may be obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (see, e.g., WO 86/05807; WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulfhydyl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCR based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g., humanized antibodies.

Once a nucleic acid encoding an antibody molecule of the invention has been obtained, the vector for the production of the antibody molecule may be produced by recombinant DNA technology using techniques well known in the art. Thus, methods for preparing the protein of the invention by expressing nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2d Ed., Cold Spring Harbor Laboratory, Cold Spring Harbor, N.Y.) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus is an effective expression system for antibodies (Foecking et al., 198, Gene 45: 101; Cockett et al., 1990, Bio/Technology 8:2).

A variety of host-expression vector systems may be utilized to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, HEK293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g., an adenovirus expression system) may be utilized.

As discussed above, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of recombinant antibodies, stable expression is preferred. For example, cell lines that stably express an antibody of interest can be produced by transfecting the cells with an expression vector comprising the nucleotide sequence of the antibody and the nucleotide sequence of a selectable marker (e.g., neomycin or hygromycin), and selecting for expression of the selectable marker. Such engineered cell lines may be particularly useful in screening and evaluation of agents that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once the antibody molecule of the invention has been recombinantly expressed, it may be purified by any method known in the art for purification of an antibody molecule, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni²⁺ nitriloacetic acid-agarose columns and histidine-tagged proteins are selectively eluted with imidazole-containing buffers.

Conjugated Antibodies

In a preferred embodiment, anti-EOPI antibodies or fragments thereof are conjugated to a diagnostic or therapeutic moiety. The antibodies can be used for diagnosis or to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

Anti-EOPI antibodies or fragments thereof can be conjugated to a therapeutic agent or drug moiety to modify a given biological response. The therapeutic agent or drug moiety is not to be construed as limited to classical chemical therapeutic agents, e.g., small molecules. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator, a thrombotic agent or an anti-angiogenic agent, e.g., angiostatin or endostatin; or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor (NGF) or other growth factor.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabelled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). These references are incorporated herein in their entirety.

Alternatively, an antibody can be conjugated to a second antibody to form an antibody heteroconjugate as described in U.S. Pat. No. 4,676,980.

An antibody with or without a therapeutic moiety conjugated to it can be used as a therapeutic that is administered alone or in combination with cytokine(s).

Diagnosis of ErbB2 Related Cancer

In accordance with the present invention, a first suitable sample e.g. tissue, serum, plasma or urine obtained from a subject suspected of having or known to have ErbB2 related cancer can be used for diagnosis or monitoring. In one embodiment, a decreased abundance of one or more EOFs or EOPIs (or any combination of them) in a first sample or sample set relative to a second sample or sample set (from a subject or subjects free from ErbB2 related cancer) or a previously determined reference range indicates the presence of ErbB2 related cancer; EOFs and EOPIs suitable for this purpose are identified in Lists I-II and V-VI respectively and described in Tables I and III respectively, as detailed above. In another embodiment of the invention, an increased abundance of one or more EOFs or EOPIs (or any combination of them) in a first sample or sample set compared to a second sample or sample set or a previously determined reference range indicates the presence of ErbB2 related cancer; EOFs and EOPIs suitable for this purpose are identified in Lists III-IV and VII-VIII, respectively and described in Tables I and III respectively, as detailed above. In another embodiment, the relative abundance of one or more EOFs or EOPIs (or any combination of them) in a first sample or sample set compared to a second sample or sample set or a previously determined reference range indicates a subtype of ErbB2 related cancer (e.g., benign or progressive ErbB2 related cancer). In yet another embodiment, the relative abundance of one or more EOFs or EOPIs (or any combination of them) in a first sample or sample set relative to a second sample or sample set or a previously determined reference range indicates the degree or severity of ErbB2 related cancer. In any of the aforesaid methods, detection of one or more EOPIs described herein may optionally be combined with detection of one or more additional biomarkers for ErbB2 related cancer including, but not limited to, oligoclonal immunoglobulin bands in tissue or body fluid revealed by isoelectric focusing (Reiber H et al. (1998) Mult Scler 3: 111-7). Any suitable method in the art can be employed to measure the level of EOFs and EOPIs, including but not limited to the Preferred Technology described herein, kinase assays, immunoassays to detect and/or visualize the EOPIs (e.g., Western blot, immunoprecipitation followed by sodium dodecyl sulfate polyacrylamide gel electrophoresis, immunocytochemistry, etc.). In cases where an EOPI has a known function, an assay for that function may be used to measure EOPI expression. In a further embodiment, a decreased abundance of mRNA including one or more EOPIs identified in Lists V and VI (or any combination of them) in a first sample or sample set relative to a second sample or sample set or a previously determined reference range indicates the presence of ErbB2 related cancer. In yet a further embodiment, an increased abundance of mRNA encoding one or more EOPIs identified in Lists VII and VIII (or any combination of them) in a first sample or sample set relative to a second sample or sample set or previously determined reference range indicates the presence of ErbB2 related cancer. Any suitable hybridization assay can be used to detect EOPI expression by detecting and/or visualizing mRNA encoding the EOPI (e.g., Northern assays, dot blots, in situ hybridization, etc.).

In another embodiment of the invention, labelled antibodies, derivatives and analogs thereof, which specifically bind to an EOPI can be used for diagnostic purposes to detect, diagnose, or monitor ErbB2 related cancer. Preferably, ErbB2 related cancer is detected in an animal, more preferably in a mammal and most preferably in a human.

Screening Assays

The invention provides methods for identifying agents (e.g., candidate agents) that bind to an EOPI or have a stimulatory or inhibitory effect on the expression or activity of an EOPI. The invention also provides methods of identifying agents or candidate agents that bind to an EOPI fragment, EOPI-related polypeptide or the EOPI-fusion protein or have a stimulatory or inhibitory effect on the expression or activity of an EOPI fragment, EOPI-related polypeptide or an EOPI fusion protein. Examples of agents or candidate agents include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12: 145; U.S. Pat. No. 5,738,996; and U.S. Pat. No. 5,807,683).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented, e.g., presented in solution (e.g., Houghten, 1992, Bio/Techniques 13:412421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404406; Cwirla et al., 1990, Proc. Natl. Acad. Sci. USA 87:6378-6382; and Felici, 1991, J. Mol. Biol. 222:301-310), each of which is incorporated herein in its entirety by reference.

In one embodiment, agents that interact with (i.e., bind to) an EOPI, an EOPI fragment (e.g. a functionally active fragment), an EOPI-related polypeptide, a fragment of an EOPI-related polypeptide, or an EOPI fusion protein are identified in a cell-based assay system. In accordance with this embodiment, cells expressing an EOPI, a fragment of an EOPI, an EOPI-related polypeptide, a fragment of an EOPI-related polypeptide, or an EOPI fusion protein are contacted with a candidate agent or a control agent and the ability of the candidate agent to interact with the EOPI is determined. If desired, this assay may be used to screen a plurality (e.g. a library) of candidate agents. The cell, for example, can be of prokaryotic origin (e.g., E. coli) or eukaryotic origin (e.g., yeast or mammalian). Further, the cells can express the EOPI, fragment of the EOPI, EOPI-related polypeptide, a fragment of the EOPI-related polypeptide, or an EOPI fusion protein endogenously or be genetically engineered to express the EOPI, fragment of the EOPI, EOPI-related polypeptide, a fragment of the EOPI-related polypeptide, or an EOPI fusion protein. In certain instances, the EOPI, fragment of the EOPI, EOPI-related polypeptide, a fragment of the EOPI-related polypeptide, or an EOPI fusion protein or the candidate agent is labelled, for example with a radioactive label (such as 32P, 35S or 125I) or a fluorescent label (such as fluorescein isothiocyanate, rhodamine, phycoerythrin, phycocyanin, allophycocyanin, o-phthaldehyde or fluorescamine) to enable detection of an interaction between an EOPI and a candidate agent. The ability of the candidate agent to interact directly or indirectly with an EOPI, a fragment of an EOPI, an EOPI-related polypeptide, a fragment of an EOPI-related polypeptide, or an EOPI fusion protein can be determined by methods known to those of skill in the art. For example, the interaction between a candidate agent and an EOPI, a fragment of an EOPI, an EOPI-related polypeptide, a fragment of an EOPI-related polypeptide, or an EOPI fusion protein can be determined by flow cytometry, a scintillation assay, immunoprecipitation or western blot analysis.

In another embodiment, agents that interact with (i.e., bind to) an EOPI, an EOPI fragment (e.g., a functionally active fragment) an EOPI-related polypeptide, a fragment of an EOPI-related polypeptide, or an EOPI fusion protein are identified in a cell-free assay system. In accordance with this embodiment, a native or recombinant EOPI or fragment thereof, or a native or recombinant EOPI-related polypeptide or fragment thereof, or an EOPI-fusion protein or fragment thereof, is contacted with a candidate agent or a control agent and the ability of the candidate agent to interact with the EOPI or EOPI-related polypeptide, or EOPI fusion protein is determined. If desired, this assay may be used to screen a plurality (e.g. a library) of candidate agents. Preferably, the EOPI, EOPI fragment, EOPI-related polypeptide, a fragment of an EOPI-related polypeptide, or an EOPI-fusion protein is first immobilized, by, for example, contacting the EOPI, EOPI fragment, EOPI-related polypeptide, a fragment of an EOPI-related polypeptide, or an EOPI fusion protein with an immobilized antibody which specifically recognizes and binds it, or by contacting a purified preparation of the EOPI, EOPI fragment, EOPI-related polypeptide, fragment of an EOPI-related polypeptide, or an EOPI fusion protein with a surface designed to bind proteins. The EOPI, EOPI fragment, EOPI-related polypeptide, a fragment of an EOPI-related polypeptide, or an EOPI fusion protein may be partially or completely purified (e.g., partially or completely free of other polypeptides) or part of a cell lysate. Further, the EOPI, EOPI fragment, EOPI-related polypeptide, a fragment of an EOPI-related polypeptide may be a fusion protein comprising the EOPI or a biologically active portion thereof, or EOPI-related polypeptide and a domain such as glutathionine-S-transferase. Alternatively, the EOPI, EOPI fragment, EOPI-related polypeptide, fragment of an EOPI-related polypeptide or EOPI fusion protein can be biotinylated using techniques well known to those of skill in the art (e.g., biotinylation kit, Pierce Chemicals; Rockford, Ill.). The ability of the candidate agent to interact with an EOPI, EOPI fragment, EOPI-related polypeptide, a fragment of an EOPI-related polypeptide, or an EOPI fusion protein can be can be determined by methods known to those of skill in the art.

In another embodiment, a cell-based assay system is used to identify agents that bind to or modulate the activity of a protein, such as an enzyme, or a biologically active portion thereof, which is responsible for the production or degradation of an EOPI or is responsible for the post-translational modification of an EOPI. In a primary screen, a plurality (e.g., a library) of candidate agents are contacted with cells that naturally or recombinantly express: (i) an EOPI, an EOPI homolog an EOPI-related polypeptide, an EOPI fusion protein, or a biologically active fragment of any of the foregoing; and (ii) a protein that is responsible for processing of the EOPI, EOPI homolog, EOPI-related polypeptide, EOPI fusion protein, or fragment in order to identify agents that modulate the production, degradation, or post-translational modification of the EOPI, EOPI homolog, EOPI-related polypeptide, EOPI fusion protein or fragment. If desired, candidate agents identified in the primary screen can then be assayed in a secondary screen against cells naturally or recombinantly expressing the specific EOPIs of interest. The ability of the candidate agent to modulate the production, degradation or post-translational modification of an EOPI, homolog, EOPI-related polypeptide, or EOPI fusion protein can be determined by methods known to those of skill in the art, including without limitation, flow cytometry, a scintillation assay, immunoprecipitation and western blot analysis.

In another embodiment, agents that competitively interact with (i.e., bind to) an EOPI, EOPI fragment, EOPI-related polypeptide, a fragment of an EOPI-related polypeptide, or an EOPI fusion protein are identified in a competitive binding assay. In accordance with this embodiment, cells expressing an EOPI, EOPI fragment, EOPI-related polypeptide, a fragment of an EOPI-related polypeptide, or an EOPI fusion protein are contacted with a candidate agent and an agent known to interact with the EOPI, EOPI fragment, EOPI-related polypeptide, a fragment of an EOPI-related polypeptide or an EOPI fusion protein; the ability of the candidate agent to competitively interact with the EOPI, EOPI fragment, EOPI-related polypeptide, fragment of an EOPI-related polypeptide, or an EOPI fusion protein is then determined. Alternatively, agents that competitively interact with (i.e., bind to) an EOPI, EOPI fragment, EOPI-related polypeptide or fragment of an EOPI-related polypeptide are identified in a cell-free assay system by contacting an EOPI, EOPI fragment, EOPI-related polypeptide, fragment of an EOPI-related polypeptide, or an EOPI fusion protein with a candidate agent and an agent known to interact with the EOPI, EOPI-related polypeptide or EOPI fusion protein. As stated above, the ability of the candidate agent to interact with an EOPI, EOPI fragment, EOPI-related polypeptide, a fragment of an EOPI-related polypeptide, or an EOPI fusion protein can be determined by methods known to those of skill in the art. These assays, whether cell-based or cell-free, can be used to screen a plurality (e.g., a library) of candidate agents.

In another embodiment, agents that modulate (i.e., upregulate or downregulate) the expression of an EOPI, or an EOPI-related polypeptide are identified by contacting cells (e.g., cells of prokaryotic origin or eukaryotic origin) expressing the EOPI, or EOPI-related polypeptide with a candidate agent or a control agent (e.g., phosphate buffered saline (PBS)) and determining the expression of the EOPI, EOPI-related polypeptide, or EOPI fusion protein, mRNA encoding the EOPI, or mRNA encoding the EOPI-related polypeptide. The level of expression of a selected EOPI, EOPI-related polypeptide, mRNA encoding the EOPI, or mRNA encoding the EOPI-related polypeptide in the presence of the candidate agent is compared to the level of expression of the EOPI, EOPI-related polypeptide, mRNA encoding the EOPI, or mRNA encoding the EOPI-related polypeptide in the absence of the candidate agent (e.g., in the presence of a control agent). The candidate agent can then be identified as a modulator of the expression of the EOPI, or an EOPI-related polypeptide based on this comparison. For example, when expression of the EOPI or mRNA is significantly greater in the presence of the candidate agent than in its absence, the candidate agent is identified as a stimulator of expression of the EOPI or mRNA. Alternatively, when expression of the EOPI or mRNA is significantly less in the presence of the candidate agent than in its absence, the candidate agent is identified as an inhibitor of the expression of the EOPI or mRNA. The level of expression of an EOPI or the mRNA that encodes it can be determined by methods known to those of skill in the art. For example, mRNA expression can be assessed by Northern blot analysis or RT-PCR, and protein levels can be assessed by western blot analysis.

The candidate agent may be an agonist or an antagonist of the EOPI, EOPI-related polypeptide, or EOPI fusion protein, or of an upstream effector of the EOPI, EOPI-related polypeptide or EOPI fusion protein.

In another embodiment, agents that modulate the activity of an EOPI, or an EOPI-related polypeptide are identified by contacting a preparation containing the EOPI or EOPI-related polypeptide, or cells (e.g., prokaryotic or eukaryotic cells) expressing the EOPI or EOPI-related polypeptide with a candidate agent or a control agent and determining the ability of candidate agent to modulate (e.g., stimulate or inhibit) the activity of the EOPI or EOPI-related polypeptide. The activity of an EOPI or an EOPI-related polypeptide can be assessed by detecting changes in a downstream effector such as, without limitation, induction of a cellular signal transduction pathway of the EOPI or EOPI-related polypeptide (e.g., intracellular Ca²⁺, diacylglycerol, IP3, etc.), detecting catalytic or enzymatic activity of the target on a suitable substrate, detecting the induction of a reporter gene (e.g., a regulatory element that is responsive to an EOPI or an EOPI-related polypeptide and is operably linked to a nucleic acid encoding a detectable marker, e.g., luciferase), or detecting a cellular response, for example, cellular differentiation, or cell proliferation. Based on the present description, techniques known to those of skill in the art can be used for measuring these activities (see, e.g., U.S. Pat. No. 5,401,639, which is incorporated herein by reference). The candidate agent can then be identified as a modulator of the activity of an EOPI or EOPI-related polypeptide by comparing the effects of the candidate agent to the control agent. Suitable control agents include phosphate buffered saline (PBS) and normal saline (NS).

In another embodiment, agents that modulate (i.e., upregulate or downregulate) the expression, activity or both the expression and activity of an EOPI or EOPI-related polypeptide are identified in an animal model. Examples of suitable animals include, but are not limited to, mice, rats, rabbits, monkeys, guinea pigs, dogs and cats Preferably, the animal used represent a model of ErbB2-related cancer (e.g., transgenic mice or rats expressing the ErbB2 oncogene, such as those described in Davies BR, Auersperg N, Worsley SD, Ponder BA, Transfection of rat ovarian surface epithelium with erb-B2/neu induces transformed phenotypes in vitro and the tumorigenic phenotype in vivo, Am J Pathol 1998 January;152(1):297-306, and Maurer-Gebhard M, Schmidt M, Azemar M, Stocklin E, Wels W, Groner B., A novel animal model for the evaluation of the efficacy of drugs directed against the ErbB2 receptor on metastasis formation, Hybridoma 1999 February;18(1):69-75). In accordance with this embodiment, the candidate agent or a control agent is administered (e.g., orally, rectally or parenterally such as intraperitoneally or intravenously) to a suitable animal and the effect on the expression, activity or both expression and activity of the EOPI or EOPI-related polypeptide is determined. Changes in the expression of an EOPI or EOPI-related polypeptide can be assessed by the methods outlined above.

In yet another embodiment, an EOPI or EOPI-related polypeptide is used as a “bait protein” in a two-hybrid assay or three hybrid assay to identify other proteins that bind to or interact with an EOPI or EOPI-related polypeptide (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et.al. (1993) Oncogene 8:1693-1696; and WO 94/10300). As those skilled in the art will appreciate, such binding proteins may be involved in the propagation of signals by the EOPIs of the invention as, for example, upstream or downstream elements of a signaling pathway involving the EOPIs of the invention.

Suitable assays can be employed for detecting or quantifying enzymatic or binding activity of an EOPI, an EOPI analog, an EOPI-related polypeptide, or a fragment of any of the foregoing. In a preferred embodiment, an assay is used to screen for or identify candidate agent that modulates the activity and or expression of an EOPI, EOPI analog, or EOPI-related polypeptide, a fragment of any of the foregoing.

One skilled in the art will also appreciate that an EOPI, an EOPI analog, an EOPI-related polypeptide, or a fragment of any of the foregoing may be used in a method for the structure-based design of an agent, in particular a small molecule which acts to modulate (e.g. stimulate or inhibit) the activity of said EOPI, EOPI analog, EOPI-related polypeptide, or fragment of any of the foregoing, said method comprising:

1) determining the three-dimensional structure of said EOPI, EOPI analog, EOPI-related polypeptide, or fragment of any of the foregoing,

2) deducing the three-dimensional structure of the likely reactive or binding site(s) of the agent,

3) synthesising candidate agents that are predicted to react or bind to the deduced reactive or binding site; and

4) testing whether the candidate agent is able to modulate the activity of said EOPI, EOPI analog, EOPI-related polypeptide, or fragment of any of the foregoing.

It will be appreciated that the method described above is likely to be an iterative process.

This invention further provides novel active agents identified by the above-described screening assays and uses thereof for treatments as described herein. In addition, the invention also provides the use of an active agent, which interacts with, or modulates the activity of an EOPI, an EOPI analog, an EOPI-related polypeptide, or a fragment of any of the foregoing in the manufacture of a composition for the treatment of ErbB2 related cancer, wherein the ErbB2 related cancer is selected from breast, ovary, stomach or lung cancer.

An important feature of the present invention is the identification of a gene encoding an EOPI, an EOPI analog, an EOPI-related polypeptide, or a fragment of any of the foregoing as defined herein involved in ErbB2 related cancer. ErbB2 related cancer can be treated or prevented by administration of an active agent that modulates activity or expression of an EOPI, an EOPI analog, an EOPI-related polypeptide, or a fragment of any of the foregoing as defined herein in the tissue of patients with an ErbB2 related cancer.

Therapeutic Uses of EOPIs

The invention provides for treatment or prevention of various diseases and disorders by administration of a therapeutic agent. Such agents include but are not limited to: EOPIs, EOPI analogs, EOPI-related polypeptides and derivatives (including fragments) thereof; antibodies to the foregoing; nucleic acids encoding EOPIs, EOPI analogs, EOPI-related polypeptides and fragments thereof; antisense nucleic acids to a gene encoding an EOPI or EOPI-related polypeptide; and modulator (e.g., agonists and antagonists) of a gene encoding an EOPI or EOPI-related polypeptide. An important feature of the present invention is the identification of genes encoding EOPIs involved in ErbB2 related cancer. ErbB2 related cancer can be treated (e.g. to ameliorate symptoms or to retard onset or progression) or prevented by administration of a therapeutic agent that promotes function or expression of one or more EOPIs that are decreased in the tissue or body fluid of ErbB2 related cancer subjects having ErbB2 related cancer, or by administration of a therapeutic agent that reduces function or expression of one or more EOPIs that are increased in the tissue or body fluid of subjects having ErbB2 related cancer.

In one embodiment, one or more antibodies each specifically binding to an EOPI are administered alone or in combination with one or more additional therapeutic agents or treatments. Examples of such therapeutic compounds or treatments include, but are not limited to, taxol, cyclophosphamide, taxomifen, fluorouracil, doxorubicin.

Preferably, a biological product such as an antibody is allogeneic to the subject to which it is administered. In a preferred embodiment, a human EOPI or a human EOPI-related polypeptide, a nucleotide sequence encoding a human EOPI or a human EOPI-related polypeptide, or an antibody to a human EOPI or a human EOPI-related polypeptide, is administered to a human subject for therapy (e.g. to ameliorate symptoms or to retard onset or progression) or prophylaxis.

Treatment and Prevention of ErbB2 Related Cancer

ErbB2 related cancer is treated or prevented by administration to a subject suspected of having or known to have ErbB2 related cancer or to be at risk of developing ErbB2 related cancer an agent that modulates (i.e., increases or decreases) the level or activity (i.e., function) of one or more EOPIs, or the level of one or more EOFs, that are differentially present in the tissue or body fluid of subjects having ErbB2 related cancer compared with tissue or body fluid of subjects free from ErbB2 related cancer. In one embodiment, ErbB2 related cancer is treated or prevented by administering to a subject suspected of having or known to have ErbB2 related cancer or to be at risk of developing ErbB2 related cancer an agent that upregulates (i.e., increases) the level or activity (i.e., function) of one or more EOPIs, or the level of one or more EOFs, that are decreased in the tissue or body fluid of subjects having ErbB2 related cancer. In another embodiment, an agent is administered that upregulates the level or activity (i.e., function) of one or more EOPIs, or the level of one or more EOFs, that are increased in the tissue or body fluid of subjects having ErbB2 related cancer. Examples of such an agent include but are not limited to: EOPIs, EOPI fragments and EOPI-related polypeptides; nucleic acids encoding an EOPI, an EOPI fragment and an EOPI-related polypeptide (e.g., for use in gene therapy); and, for those EOPIs or EOPI-related polypeptides with enzymatic activity, agents or molecules known to modulate that enzymatic activity. Other agents that can be used, e.g., EOPI agonists, can be identified using in vitro assays.

ErbB2 related cancer is also treated or prevented by administration to a subject suspected of having or known to have ErbB2 related cancer or to be at risk of developing ErbB2 related cancer an agent that downregulates the level or activity of one or more EOPIs, or the level of one or more EOFs, that are increased in the tissue or body fluid of subjects having ErbB2 related cancer. In another embodiment, an agent is administered that downregulates the level or activity of one or more EOPIs, or the level of one or more EOFs, that are decreased in the tissue or body fluid of subjects having ErbB2 related cancer. Examples of such an agent include, but are not limited to, EOPI antisense oligonucleotides, ribozymes, antibodies directed against EOPIs, and agents that inhibit the enzymatic activity of an EOPI. Other useful agents e.g., EOPI antagonists and small molecule EOPI antagonists, can be identified using in vitro assays.

In a preferred embodiment, therapy or prophylaxis is tailored to the needs of an individual subject. Thus, in specific embodiments, agents that promote the level or function of one or more EOPIs, or the level of one or more EOFs, are therapeutically or prophylactically administered to a subject suspected of having or known to have ErbB2 related cancer, in whom the levels or functions of said one or more EOPIs, or levels of said one or more EOFs, are absent or are decreased relative to a control or normal reference range. In further embodiments, agents that promote the level or function of one or more EOPIs, or the level of one or more EOFs, are therapeutically or prophylactically administered to a subject suspected of having or known to have ErbB2 related cancer in whom the levels or functions of said one or more EOPIs, or levels of said one or more EOFs, are increased relative to a control or to a reference range. In further embodiments, agents that decrease the level or function of one or more EOPIs, or the level of one or more EOFs, are therapeutically or prophylactically administered to a subject suspected of having or known to have ErbB2 related cancer in whom the levels or functions of said one or more EOPIs, or levels of said one or more EOFs, are increased relative to a control or to a reference range. In further embodiments, agents that decrease the level or function of one or more EOPIs, or the level of one or more EOFs, are therapeutically or prophylactically administered to a subject suspected of having or known to have ErbB2 related cancer in whom the levels or functions of said one or more EOPIs, or levels of said one or more EOFs, are decreased relative to a control or to a reference range. The change in EOPI function or level, or EOF level, due to the administration of such agents can be readily detected, e.g., by obtaining a sample (e.g., a sample of, blood or urine or a tissue sample such as biopsy tissue) and assaying in vitro the levels of said EOFs or the levels or activities of said EOPIs, or the levels of mRNAs encoding said EOPIs or any combination of the foregoing. Such assays can be performed before and after the administration of the agent as described herein.

The agents of the invention include but are not limited to any agent, e.g., a small organic molecule, protein, peptide, antibody, nucleic acid, etc. that restores the ErbB2 related cancer EOPI or EOF profile towards normal with the proviso that such agents do not include: cyclophophamide (Cytoxan T); methotrexate (Methotrexate™); 5-fluorouracil (5-FU); paclitaxel (Taxol™); docetaxel (Taxotere™); vincristine (Oncovin™); vinblastine (Velban™); vinorelbine (Navelbine™); doxorubicin (Adriamycin™); tamoxifen (Nolvadex™); toremifene(Fareston™); megestrol acetate(Megace™); anastrozole (Arimidex™); goserelin (Zoladex™); trastuzumab (Herceptin™); (Taxomifen™); capecitabine (Xeloda™.).

Gene Therapy

In a specific embodiment, nucleic acids comprising a sequence encoding an EOPI, an EOPI fragment, EOPI-related polypeptide or fragment of an EOPI-related polypeptide, are administered to promote EOPI function by way of gene therapy. Gene therapy refers to administration to a subject of an expressed or expressible nucleic acid. In this embodiment, the nucleic acid produces its encoded polypeptide that mediates a therapeutic effect by promoting EOPI function.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5): 155-215). Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred aspect, the agent comprises a nucleic acid encoding an EOPI or fragment or chimeric protein thereof, said nucleic acid being part of an expression vector that expresses an EOPI or fragment or chimeric protein thereof in a suitable host. In particular, such a nucleic acid has a promoter operably linked to the EOPI coding region, said promoter being inducible or constitutive (and, optionally, tissue-specific). In another particular embodiment, a nucleic acid molecule is used in which the EOPI coding sequences and any other desired sequences are flanked by regions that promote homologous recombination at a desired site in the genome, thus providing for intrachromosomal expression of the EOPI nucleic acid (Koller and Smithies, 1989, Proc. Natl. Acad. Sci. USA 86:8932-8935; Zijlstra et al., 1989, Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject; this approach is known as ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic™, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., WO 92/06180, WO 92/22635, WO92/20316, WO93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, a viral vector that contains a nucleic acid encoding an EOPI is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The nucleic acid encoding the EOPI to be used in gene therapy is cloned into the vector, which facilitates delivery of the gene into a subject. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; WO94/12649; and Wang, et al., 1995, Gene Therapy 2:775-783.

Adeno-associated virus (AAV) has also been proposed for use in gene therapy (Walsh et al., 1993, Proc. Soc. Exp. Biol. Med. 204:289-300; U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject. Recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver.

In a preferred embodiment, the cell used for gene therapy is autologous to the subject that is treated.

In an embodiment in which recombinant cells are used in gene therapy, a nucleic acid encoding an EOPI is introduced into the cells such that it is expressible by the cells or their progeny, and the recombinant cells are then administered in vivo for therapeutic effect. In a specific embodiment, stem or progenitor cells are used. Any stem or progenitor cells which can be isolated and maintained in vitro can be used in accordance with this embodiment of the present invention (see e.g. WO 94/08598; Stemple and Anderson, 1992, Cell 71:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Direct injection of a DNA coding for an EOPI may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection and the elicitation of an immune response in the subject to the protein encoded by the injected DNA. In a preferred embodiment, naked DNA comprising (a) DNA encoding an EOPI and (b) a promoter are injected into a subject to elicit an immune response to the EOPI.

Inhibition of EOPIs to Treat ErbB2 Related Cancer

In one embodiment of the invention, ErbB2 related cancer is treated or prevented by administration of an agent that antagonizes (inhibits) the level(s) and/or function(s) of one or more EOPIs which are elevated in a sample of subjects having ErbB2 related cancer as compared with a sample of subjects free from ErbB2 related cancer. Agents useful for this purpose include but are not limited to anti-EOPI antibodies (and fragments and derivatives containing the binding region thereof), EOPI antisense or ribozyme nucleic acids, and nucleic acids encoding dysfunctional EOPIs that are used to “knockout” endogenous EOPI function by homologous recombination (see, e.g., Capecchi, 1989, Science 244:1288-1292). Other agents that inhibit EOPI function can be identified by use of known in vitro assays, e.g., assays for the ability of a test agent to inhibit binding of an EOPI to another protein or a binding partner, or to inhibit a known EOPI function. Preferably such inhibition is assayed in vitro or in cell culture, but genetic assays may also be employed. The Preferred Technology can also be used to detect levels of the EOPIs before and after the administration of the agent. Preferably, suitable in vitro or in vivo assays are utilized to determine the effect of a specific agent and whether its administration is indicated for treatment of the affected tissue, as described in more detail below.

In a specific embodiment, an agent that inhibits an EOPI function is administered therapeutically or prophylactically to a subject in whom an increased tissue or body fluid level or functional activity of the EOPI (e.g., greater than the normal level or desired level) is detected as compared with tissue or body fluid of subjects free from ErbB2 related cancer or a predetermined reference range. Methods standard in the art can be employed to measure the increase in an EOPI level or function, as outlined above. Preferred EOPI inhibitor compositions include small molecules, i.e., molecules of 1000 daltons or less. Such small molecules can be identified by the screening methods described herein.

In a further embodiment, an EOPI may be seen to be decreased in the tissue or body fluid where this decrease represents an increase in the EOPI level in another compartment, for example but without limitation sequestering of the EOPI in a cell, subcellular compartment, body fluid or tissue rather than secretion into tissue or body fluid. Under these conditions, an agent that inhibits an EOPI function is administered therapeutically or prophylactically to a subject in whom a decreased tissue or body fluid level or functional activity of the EOPI (e.g., greater than the normal level or desired level) is detected as compared with tissue or body fluid of subjects free from ErbB2 related cancer or a predetermined reference range.

Antisense Regulation of EOPIs

In a specific embodiment, EOPI expression is inhibited by use of EOPI antisense nucleic acids. The present invention provides the therapeutic or prophylactic use of nucleic acids comprising at least six nucleotides that are antisense to a gene or cDNA encoding an EOPI or a portion thereof. As used herein, an EOPI “antisense” nucleic acid refers to a nucleic acid capable of hybridizing by virtue of some sequence complementarity to a portion of an RNA (preferably mRNA) encoding an EOPI. The antisense nucleic acid may be complementary to a coding and/or noncoding region of an mRNA encoding an EOPI. Such antisense nucleic acids have utility as agents that inhibit EOPI expression, and can be used in the treatment or prevention of ErbB2 related cancer.

The antisense nucleic acids of the invention are double-stranded or single-stranded oligonucleotides, RNA or DNA or a modification or derivative thereof, and can be directly administered to a cell or produced intracellularly by transcription of exogenous, introduced sequences.

The invention further provides pharmaceutical compositions comprising an effective amount of the EOPI antisense nucleic acids of the invention in a pharmaceutically acceptable carrier, as described infra.

In another embodiment, the invention provides methods for inhibiting the expression of an EOPI nucleic acid sequence in a prokaryotic or eukaryotic cell comprising providing the cell with an effective amount of a composition comprising an EOPI antisense nucleic acid of the invention.

EOPI antisense nucleic acids and their uses are described in detail below.

EOPI Antisense Nucleic Acids

The EOPI antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. The oligonucleotide can be modified at the base moiety, sugar moiety, or phosphate backbone. The oligonucleotide may include other appended groups such as peptides; agents that facilitate transport across the cell membrane (see, e.g., Letsinger et al., 1989, Proc. Natl. Acad. Sci. USA 86:6553-6556; Lemaitre et al., 1987, Proc. Natl. Acad. Sci. 84:648-652; WO 88/09810,) or blood-brain barrier (see, e.g., WO 89/10134,); hybridization-triggered cleavage agents (see, e.g., Krol et al., 1988, BioTechniques 6:958-976) or intercalating agents (see, e.g., Zon, 1988, Pharm. Res. 5:539-549).

In a preferred aspect of the invention, an EOPI antisense oligonucleotide is provided, preferably of single-stranded DNA. The oligonucleotide may be modified at any position on its structure with substituents generally known in the art.

The EOPI antisense oligonucleotide may comprise at least one of the following modified base moieties: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, 2,6-diaminopurine, and other base analogs.

In another embodiment, the oligonucleotide comprises at least one modified sugar moiety, e.g., one of the following sugar moieties: arabinose, 2-fluoroarabinose, xylulose, and hexose.

In yet another embodiment, the oligonucleotide comprises at least one of the following modified phosphate backbones: a phosphorothioate, a phosphorodithioate, a phosphoramidothioate, a phosphoramidate, a phosphordiamidate, a methylphosphonate, an alkyl phosphotriester, a formacetal, or an analog of formacetal.

In yet another embodiment, the oligonucleotide is an alpha-anomeric oligonucleotide. An alpha-anomeric oligonucleotide forms specific double-stranded hybrids with complementary RNA in which, contrary to the usual alpha-units, the strands run parallel to each other (Gautier et al., 1987, Nucl. Acids Res. 15:6625-6641).

The oligonucleotide may be conjugated to another molecule, e.g., a peptide, hybridization triggered cross-linking agent, transport agent, or hybridization-triggered cleavage agent.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), and methylphosphonate oligonucleotides can be prepared by use of controlled pore glass polymer supports (Sarin et al., 1988, Proc. Natl. Acad. Sci. USA 85:7448-7451).

In a specific embodiment, the EOPI antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the EOPI antisense nucleic acid. Such a vector can remain episomal or become chromosomally integrated, as long as it can be transcribed to produce the desired antisense RNA. Such vectors can be constructed by recombinant DNA technology standard in the art. Vectors can be plasmid, viral, or others known in the art, used for replication and expression in mammalian cells. Expression of the sequence encoding the EOPI antisense RNA can be by any promoter known in the art to act in mammalian, preferably human, cells. Such promoters can be inducible or constitutive. Examples of such promoters are outlined above.

The antisense nucleic acids of the invention comprise a sequence complementary to at least a portion of an RNA transcript of a gene encoding an EOPI, preferably a human gene encoding an EOPI. However, absolute complementarity, although preferred, is not required. A sequence “complementary to at least a portion of an RNA,” as referred to herein, means a sequence having sufficient complementarity to be able to hybridize under stringent conditions (e.g., highly stringent conditions or moderately stringent conditions as defined supra) with the RNA, forming a stable duplex; in the case of double-stranded EOPI antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA encoding an EOPI it may contain and still form a stable duplex (or triplex, as the case may be). One skilled in the art can ascertain a tolerable degree of mismatch by use of standard procedures to determine the melting point of the hybridized complex.

Therapeutic Use of EOPI Antisense Nucleic Acids

The EOPI antisense nucleic acids can be used to treat or prevent ErbB2 related cancer when the target EOPI is overexpressed in the tissue or body fluid of subjects suspected of having or suffering from ErbB2 related cancer. In a preferred embodiment, a single-stranded DNA antisense EOPI oligonucleotide is used.

Cell types which express or overexpress RNA encoding an EOPI can be identified by various methods known in the art. Such cell types include but are not limited to leukocytes (e.g., neutrophils, macrophages, monocytes) and resident cells (e.g., astrocytes, glial cells, neuronal cells, and ependymal cells). Such methods include, but are not limited to, hybridization with an EOPI-specific nucleic acid (e.g., by Northern hybridization, dot blot hybridization, in situ hybridization), observing the ability of RNA from the cell type to be translated in vitro into an EOPI, immunoassay, etc. In a preferred aspect, primary tissue from a subject can be assayed for EOPI expression prior to treatment, e.g., by immunocytochemistry or in situ hybridization.

Pharmaceutical compositions of the invention, comprising an effective amount of an EOPI antisense nucleic acid in a pharmaceutically acceptable carrier, can be administered to a subject having ErbB2 related cancer.

The amount of EOPI antisense nucleic acid which will be effective in the treatment of ErbB2 related cancer can be determined by standard clinical techniques.

In a specific embodiment, pharmaceutical compositions comprising one or more EOPI antisense nucleic acids are administered via liposomes, microparticles, or microcapsules. In various embodiments of the invention, such compositions may be used to achieve sustained release of the EOPI antisense nucleic acids.

Inhibitory Ribozyme and Triple Helix Approaches

In another embodiment, symptoms of ErbB2 related cancer may be ameliorated by decreasing the level of an EOPI or EOPI activity by using gene sequences encoding the EOPI in conjunction with well-known gene “knock-out,” ribozyme or triple helix methods to decrease gene expression of an EOPI. In this approach ribozyme or triple helix molecules are used to modulate the activity, expression or synthesis of the gene encoding the EOPI, and thus to ameliorate the symptoms of ErbB2 related cancer. Such molecules may be designed to reduce or inhibit expression of a mutant or non-mutant target gene. Techniques for the production and use of such molecules are well known to those of skill in the art.

Ribozyme molecules designed to catalytically cleave gene mRNA transcripts encoding an EOPI can be used to prevent translation of target gene mRNA and, therefore, expression of the gene product. (See, e.g., WO90/11364; Sarver et al., 1990, Science 247:1222-1225).

Ribozymes are enzymatic RNA molecules capable of catalyzing the specific cleavage of RNA (For a review, see Rossi, 1994, Current Biology 4, 469471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

While ribozymes that cleave mRNA at site specific recognition sequences can be used to destroy mRNAs encoding an EOPI, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334, 585-591, each of which is incorporated herein by reference in its entirety.

Preferably the ribozyme is engineered so that the cleavage recognition site is located near the 5′ end of the mRNA encoding the EOPI, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224, 574-578; Zaug and Cech, 1986, Science, 231, 470475; Zaug, et al., 1986, Nature, 324, 429433; published International patent application No. WO 88/04300 by University Patents Inc.; Been and Cech, 1986, Cell, 47, 207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the gene encoding the EOPI.

As in the antisense approach, the ribozymes can be composed of modified oligonucleotides (e.g., for improved stability, targeting, etc.) and should be delivered to cells that express the EOPI in vivo. A preferred method of delivery involves using a DNA construct “encoding” the ribozyme under the control of a strong constitutive pol III or pol II promoter, so that transfected cells will produce sufficient quantities of the ribozyme to destroy endogenous mRNA encoding the EOPI and inhibit translation. Because ribozymes, unlike antisense molecules, are catalytic, a lower intracellular concentration is required for efficacy.

Endogenous EOPI expression can also be reduced by inactivating or “knocking out” the gene encoding the EOPI, or the promoter of such a gene, using targeted homologous recombination (e.g., see Smithies, et al., 1985, Nature 317:230-234; Thomas and Capecchi, 1987, Cell 51:503-512; Thompson et al., 1989, Cell 5:313-321; and Zijlstra et al., 1989, Nature 342:435-438, each of which is incorporated by reference herein in its entirety). For example, a mutant gene encoding a non-functional EOPI (or a completely unrelated DNA sequence) flanked by DNA homologous to the endogenous gene (either the coding regions or regulatory regions of the gene encoding the EOPI) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra).

Alternatively, the endogenous expression of a gene encoding an EOPI can be reduced by targeting deoxyribonucleotide sequences complementary to the regulatory region of the gene (i.e., the gene promoter and/or enhancers) to form triple helical structures that prevent transcription of the gene encoding the EOPI in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

In instances wherein the antisense, ribozyme, or triple helix molecules described herein are utilized to inhibit mutant gene expression, it is possible that the technique may so efficiently reduce or inhibit the transcription (triple helix) or translation (antisense, ribozyme) of mRNA produced by normal gene alleles of an EOPI that the situation may arise wherein the concentration of EOPI present may be lower than is necessary for a normal phenotype. In such cases, to ensure that substantially normal levels of activity of a gene encoding an EOPI are maintained, gene therapy may be used to introduce into cells nucleic acid molecules that encode and express the EOPI that exhibit normal gene activity and that do not contain sequences susceptible to whatever antisense, ribozyme, or triple helix treatments are being utilized. Alternatively, in instances whereby the gene encodes an extracellular protein, normal EOPIs can be co-administered in order to maintain the requisite level of EOPI activity.

Antisense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Therapeutic and Prophylactic Compositions and Their Use

The invention provides methods of treatment (and prophylaxis) comprising administering to a subject an effective amount of an active agent. An “active agent” as used herein comprises EOPIs, EOPI fragments, EOPI-related polypeptides, anti-EOPI antibodies, fragments of anti-EOPI antibodes and agents which modulate the expression of EOPIs e.g. agonists and antagonists of EOPIs. In a preferred aspect, the agent is substantially purified (e.g., substantially free from substances that limit its effect or produce undesired side-effects). The subject is preferably an animal and is preferably a mammal, and most preferably human.

Formulations and methods of administration that can be employed when the agent comprises a nucleic acid are described above; additional appropriate formulations and routes of administration are described below. A “pharmaceutical composition” as used herein comprises an active agent optionally with a pharmaceutically acceptable carrier.

Various delivery systems are known and can be used to administer an agent of the invention, e.g., encapsulation in liposomes, microparticles, microcapsules, recombinant cells capable of expressing the agent, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into tissue or body fluid or at the site (or former site) of cancerous tissue.

In another embodiment, the agent can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the agent can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, e.g. near the site of cancerous tissue for example, breast, thus requiring only a fraction of the systemic dose (see, e.g., Goodson, in Medical Applications of Controlled Release, supra, vol. 2, pp. 115-138 (1984)).

Other controlled release systems are discussed in the review by Langer (1990, Science 249:1527-1533).

In a specific embodiment where the agent of the invention is a nucleic acid encoding a protein, the nucleic acid can be administered in vivo as described supra.

The present invention also provides pharmaceutical compositions. Such compositions comprise a therapeutically effective amount of an agent, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences”, Ed. E. W. Martin, ISBN: 0-912734-04-3, Mack Publishing Co. Such compositions will contain a therapeutically effective amount of the agent, preferably in purified form, together with a suitable amount of carrier so as to provide the form for proper administration to the subject. The formulation should suit the mode of administration.

In a specific embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the agent of the invention which will be effective in the treatment of ErbB2 related cancer can be determined by standard clinical techniques. In addition, in vitro assays may optionally be employed to help identify optimal dosage ranges. The precise dose to be employed in the formulation will also depend on the active agent, the route of administration of the active agent, and the seriousness of the disease or disorder, and should be decided according to the judgment of the practitioner and each subject's circumstances. However, suitable dosage ranges for intravenous administration are generally about 20-500 micrograms of active agent per kilogram body weight. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

The invention provides for the treatment of ErbB2-related cancer, wherein the ErbB2 related cancer is selected from breast, ovarian, stomach or lung cancer. Preferably, the invention provides for the treatment of breast cancer.

When a reference is made herein to a method of treating or preventing a disease or condition using a particular agent or combination of agents, it is to be understood that such a reference is intended to include the use of that agent or combination of agents in the preparation of a medicament for the treatment or prevention of the disease or condition.

Preferred features of each aspect of the invention are as for each of the other aspects mutatis mutandis.

### EXAMPLE

### Identification of Proteins Differentially Expressed in Cell Lines Overexpressing ErbB2

Using the following exemplary and non-limiting procedure, proteins in triplicate cell lysate samples taken from two cell lines overexpressing ErbB2 at different levels and one control cell line were separated by isoelectric focusing followed by SDS-PAGE and analyzed. Parts 6.1.1 to 6.1.14 (inclusive) of the procedure set forth below are hereby designated as the “Reference Protocol”.

Materials and Methods

Sample Preparation

The control cell line H4.1 and the moderately and highly ErbB2-overexpressing cell lines, C3.6 and C5.2, respectively, were derived and cultured as described in Harris et al. (Harris R A, Eichholtz T J, Hiles I D, Page M J, O'Hare M J, New model of ErbB-2 over-expression in human mammary luminal epithelial cells, Int J Cancer, 1999 Jan. 29;80(3):477-84). Each cell line was grown under identical conditions, washed with PBS (Gibco 14190-201), then lysed in OGS 2D sample buffer containing protease inhibitors (Sigma P2714).

OGS 2D sample buffer

8M urea (BDH 452043w)

2M thiourea (Fluka 88810)

4% CHAPS (Sigma C3023)

65 mM dithiotheitol (DTT)

This mixture was vortexed, and centrifuged at 13000 rpm for 5 mins at 15° C. and stored at −70° C. to await further analysis by 2D PAGE. An aliquot of 120 micrograms of the stored sample was prepared for 2D analysis by adding Resolytes 3.5-10 (BDH 44338 2×) to 2% (v/v), as well as a trace of Bromophenol Blue and further 2D sample buffer in a final volume of 370 microl.

Isoelectric Focusing

Isoelectric focusing (IEF), was performed using the Immobiline™ DryStrip Kit (Pharmacia BioTech), following the procedure described in the manufacturer's instructions, see Instructions for Immobiline™ DryStrip Kit, Pharmacia, # 18-1038-63, Edition AB (incorporated herein by reference in its entirety). Immobilized pH Gradient (IPG) strips (18 cm, pH 3-10 non-linear strips; Pharmacia Cat. # 17-1235-01) were rehydrated overnight at 20° C. in a solution of 8M urea, 2% (w/v) CHAPS, 10 mM DTT, 2% (v/v) Resolytes 3.5-10, as described in the Immobiline DryStrip Users Manual. For IEF, 50 ml of supernatant (prepared as above) was loaded onto a strip, with the cup-loading units being placed at the basic end of the strip. The loaded gels were then covered with mineral oil (Pharmacia 17-3335-01) and a voltage was immediately applied to the strips according to the following profile, using a Pharmacia EPS3500XL power supply (Cat 19-3500-01):

Initial voltage=300V for 2 hrs

Linear Ramp from 300V to 3500V over 3 hrs

Hold at 3500V for 19 hrs

For all stages of the process, the current limit was set to 10 mA for 12 gels, and the wattage limit to SW. The temperature was held at 20° C. throughout the run.

Gel Equilibration and SDS-PAGE

After the final 19 hr step, the strips were immediately removed and immersed for 10 mins at 20° C. in a first solution of the following composition: 6M urea; 2% (w/v) DTT; 2% (w/v) SDS; 30% (v/v) glycerol (Fluka 49767); 0.05M Tris/HCl, pH 6.8 (Sigma Cat T-1503). The strips were removed from the first solution and immersed for 10 mins at 20° C. in a second solution of the following composition: 6M urea; 2% (w/v) iodoacetamide (Sigma 1-6125); 2% (w/v) SDS; 30% (v/v) glycerol; 0.05M Tris/HCl, pH 6.8. After removal from the second solution, the strips were loaded onto supported gels for SDS-PAGE according to Hochstrasser et al., 1988, Analytical Biochemistry 173: 412-423 (incorporated herein by reference in its entirety), with modifications as specified below.

Preparation of Supported Gels

The gels were cast between two glass plates of the following dimensions: 23 cm wide×24 cm long (back plate); 23 cm wide×24 cm long with a 2 cm deep notch in the central 19 cm (front plate). To promote covalent attachment of SDS-PAGE gels, the back plate was treated with a 0.4% solution of g-methacryl-oxypropyltrimethoxysilane in ethanol (BindSilane™; Pharmacia Cat. # 1,7-1330-01). The front plate was treated with a 2% solution of dimethyldichlorosilane dissolved in octamethyl cyclo-octasilane (RepelSilane™ Pharmacia Cat. # 17-1332-01) to reduce adhesion of the gel. Excess reagent was removed by washing with water, and the plates were allowed to dry. At this stage, both as identification for the gel, and as a marker to identify the coated face of the plate, an adhesive bar-code was attached to the back plate in a position such that it would not come into contact with the gel matrix.

The dried plates were assembled into a casting box with a capacity of 13 gel sandwiches. The top and bottom plates of each sandwich were spaced by means of 1 mm thick spacers, 2.5 cm wide. The sandwiches were interleaved with acetate sheets to facilitate separation of the sandwiches after gel polymerization. Casting was then carried out according to Hochstrasser et al., op. cit.

A 9-16% linear polyacrylamide gradient was cast, extending up to a point 2 cm below the level of the notch in the front plate, using the Angelique gradient casting system (Large Scale Biology). Stock solutions were as follows. Acrylamide (40% in water) was from Serva (Cat. # 10677). The cross-linking agent was PDA (BioRad 161-0202), at a concentration of 2.6% (w/w) of the total starting monomer content. The gel buffer was 0.375M Tris/HCl, pH 8.8. The polymerization catalyst was 0.05% (v/v) TEMED (BioRad 161-0801), and the initiator was 0.1% (w/v) APS (BioRad 161-0700). No SDS was included in the gel and no stacking gel was used. The cast gels were allowed to polymerize at 20° C. overnight, and then stored at 4° C. in sealed polyethylene bags with 6 ml of gel buffer, and were used within 4 weeks.

SDS-PAGE

A solution of 0.5% (w/v) agarose (Fluka Cat 05075) was prepared in running buffer (0.025M Tris, 0.198M glycine (Fluka 50050), 1% (w/v) SDS, supplemented by a trace of bromophenol blue). The agarose suspension was heated to 70° C. with stirring, until the agarose had dissolved. The top of the supported 2nd D gel was filled with the agarose solution, and the equilibrated strip was placed into the agarose, and tapped gently with a palette knife until the gel was intimately in contact with the 2nd D gel. The gels were placed in the 2nd D running tank, as described by Amess et al., 1995, Electrophoresis 16: 1255-1267 (incorporated herein by reference in its entirety). The tank was filled with running buffer (as above) until the level of the buffer was just higher than the top of the region of the 2nd D gels which contained polyacrylamide, so as to achieve efficient cooling of the active gel area. Running buffer was added to the top buffer compartments formed by the gels, and then voltage was applied immediately to the gels using a Consort E-833 power supply. For 1 hour, the gels were run at 20 mA/gel. The wattage limit was set to 150W for a tank containing 6 gels, and the voltage limit was set to 600V. After 1 hour, the gels were then run at 40 mA/gel, with the same voltage and wattage limits as before, until the bromophenol blue line was 0.5 cm from the bottom of the gel. The temperature of the buffer was held at 16° C. throughout the run. Gels were not run in duplicate.

Staining

Upon completion of the electrophoresis run, the gels were immediately removed from the tank for fixation. The top plate of the gel cassette was carefully removed, leaving the gel bonded to the bottom plate. The bottom plate with its attached gel was then placed into a staining apparatus, which can accommodate 12 gels. The gels were completely immersed in fixative solution of 40% (v/v) ethanol (BDH 28719), 10% (v/v) acetic acid (BDH 100016×), 50% (v/v) water (MilliQ-Millipore), which was continuously circulated over the gels. After an overnight incubation, the fixative was drained from the tank, and the gels were primed by immersion in 7.5% (v/v) acetic acid, 0.05% (w/v) SDS, 92.5% (v/v) water for 30 mins. The priming solution was then drained, and the gels were stained by complete immersion for 4 hours in a staining solution of Pyridinium, 4-[2-[4-(dipentylamino)-2-trifluoromethylphenyl] ethenyl]-1-(sulfobutyl)-, inner salt, prepared by diluting a stock solution of this dye (2 mg/ml in DMSO) in 7.5% (v/v) aqueous acetic acid to give a final concentration of 1.2 mg/l; the staining solution was vacuum filtered through a 0.4 μm filter (Duropore) before use.

Imaging of the Gel

A computer-readable output was produced by imaging the fluorescently stained gels with the preferred scanner (Oxford Glycosciences, Oxford, UK described in section 5.2, supra. This scanner has a gel carrier with four integral fluorescent markers (Designated M1, M2, M3, M4) that are used to correct the image geometry and are a quality control feature to confirm that the scanning has been performed correctly.

For scanning, the gels were removed from the stain, rinsed with water and allowed to air dry briefly, and imaged on the preferred scanner. After imaging, the gels were sealed in polyethylene bags containing a small volume of staining solution, and then stored at 4° C.

Digital Analysis of the Data

The data were processed as described in WO 98/23950 at Sections 5.4 and 5.5 (incorporated herein by reference), as set forth more particularly below.

The output from the scanner was first processed using the MELANIE® II 2D PAGE analysis program (Release 2.2, 1997, BioRad Laboratories, Hercules, Calif., Cat. # 170-7566) to autodetect the registration points, M1, M2, M3 and M4; to autocrop the images (i.e., to eliminate signals originating from areas of the scanned image lying outside the boundaries of the gel, e.g. the reference frame); to filter out artifacts due to dust; to detect and quantify features; and to create image files in GIF format. Features were detected using the following parameters:

Smooths=2

Laplacian threshold 50

Partials threshold 1

Saturation=100

Peakedness=0

Minimum Perimeter=10

Assignment of pI and MW Values

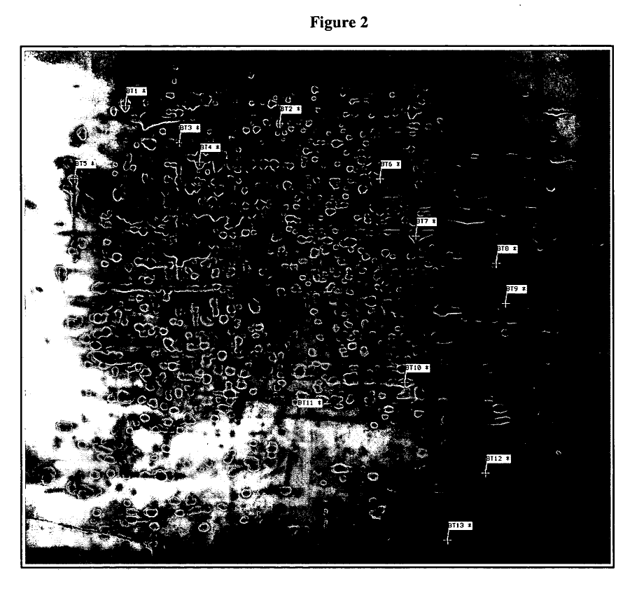

Landmark identification was used to determine the pI and MW of features detected in the images. Thirteen landmark features, designated BT1 to BT13, were identified in a standard cell line lysate image. These landmark features are identified in FIG. 2 and were assigned the pI and/or MW values identified in Table V.

TABLE V
Landmark Features Used in this Study

| Name | pl | MW (Da) |
|---|---|---|
| BT1 | 4.78 | 116,528 |
| BT2 | 5.85 | 104,386 |
| BT3 | 5.16 | 80,625 |
| BT4 | 5.36 | 71,346 |
| BT5 | 4.49 | — |
| BT6 | 6.72 | 62,494 |
| BT7 | 7.53 | 49,018 |
| BT8 | — | 42,262 |
| BT9 | 9.41 | 35,668 |
| BT10 | — | 27,823 |
| BT11 | 6.18 | 23,764 |
| BT12 | 8.72 | 15,481 |
| BT13 | 8.14 | 10,142 |

As many of these landmarks as possible were identified in each gel image of the dataset. Each feature in the study gels was then assigned a pI value by linear interpolation or extrapolation (using the MELANIE®-II software) to the two nearest landmarks, and was assigned a MW value by linear interpolation or extrapolation (using the MELANIE®-II software) to the two nearest landmarks.

Matching With Primary Master Image

Images were edited to remove gross artifacts such as dust, to reject images which had gross abnormalities such as smearing of protein features, or were of too low a loading or overall image intensity to allow identification of more than the most intense features, or were of too poor a resolution to allow accurate detection of features. Images were then compared by pairing with one common image from the whole sample set. This common image, the “primary master image”, was selected on the basis of protein load (maximum load consistent with maximum feature detection), a well resolved myoglobin region, (myoglobin was used as an internal standard), and general image quality. Additionally, the primary master image was chosen to be an image which appeared to be generally representative of all those to be included in the analysis. (This process by which a primary master gel was judged to be representative of the study gels was rechecked by the method described below and in the event that the primary master gel was seen to be unrepresentative, it was rejected and the process repeated until a representative primary master gel was found.)

Each of the remaining study gel images was individually matched to the primary master image such that common protein features were paired between the primary master image and each individual study gel image as described below.

Cross-Matching Between Samples

To facilitate statistical analysis of large numbers of samples for purposes of identifying features that are differentially expressed, the geometry of each study gel was adjusted for maximum alignment between its pattern of protein features, and that of the primary master, as follows. Each of the study gel images was individually transformed into the geometry of the primary master image using a multi-resolution warping procedure. This procedure corrects the image geometry for the distortions brought about by small changes in the physical parameters of the electrophoresis separation process from one sample to another. The observed changes are such that the distortions found are not simple geometric distortions, but rather a smooth flow, with variations at both local and global scale.

The fundamental principle in multi-resolution modeling is that smooth signals may be modeled as an evolution through ‘scale space’, in which details at successively finer scales are added to a low resolution approximation to obtain the high resolution signal. This type of model is applied to the flow field of vectors (defined at each pixel position on the reference image) and allows flows of arbitrary smoothness to be modeled with relatively few degrees of freedom. Each image is first reduced to a stack, or pyramid, of images derived from the initial image, but smoothed and reduced in resolution by a factor of 2 in each direction at every level (Gaussian pyramid) and a corresponding difference image is also computed at each level, representing the difference between the smoothed image and its progenitor (Laplacian pyramid). Thus the Laplacian images represent the details in the image at different scales.

To estimate the distortion between any 2 given images, a calculation was performed at level 7 in the pyramid (i.e. after 7 successive reductions in resolution). The Laplacian images were segmented into a grid of 16×16 pixels, with 50% overlap between adjacent grid positions in both directions, and the cross correlation between corresponding grid squares on the reference and the test images was computed. The distortion displacement was then given by the location of the maximum in the correlation matrix. After all displacements had been calculated at a particular level, they were interpolated to the next level in the pyramid, applied to the test image, and then further corrections to the displacements were calculated at the next scale.

The warping process brought about good alignment between the common features in the primary master image, and the images for the other samples. The MELANIE® II 2D PAGE analysis program was used to calculate and record approximately 500-700 matched feature pairs between the primary master and each of the other images. The accuracy of this program was significantly enhanced by the alignment of the images in the manner described above. To improve accuracy still further, all pairings were finally examined by eye in the MelView interactive editing program and residual recognizably incorrect pairings were removed. Where the number of such recognizably incorrect pairings exceeded the overall reproducibility of the Preferred Technology (as measured by repeat analysis of the same biological sample) the gel selected to be the primary master gel was judged to be insufficiently representative of the study gels to serve as a primary master gel. In that case, the gel chosen as the primary master gel was rejected, and different gel was selected as the primary master gel, and the process was repeated.

All the images were then added together to create a composite master image, and the positions and shapes of all the gel features of all the component images were super-imposed onto this composite master as described below.

Once all the initial pairs had been computed, corrected and saved, a second pass was performed whereby the original (unwarped) images were transformed a second time to the geometry of the primary master, this time using a flow field computed by smooth interpolation of the multiple tie-points defined by the centroids of the paired gel features. A composite master image was thus generated by initialising the primary master image with its feature descriptors. As each image was transformed into the primary master geometry, it was digitally summed pixel by pixel into the composite master image, and the features that had not been paired by the procedure outlined above were likewise added to the composite master image description, with their centroids adjusted to the master geometry using the flow field correction.

The final stage of processing was applied to the composite master image and its feature descriptors, which now represent all the features from all the images in the study transformed to a common geometry. The features were grouped together into linked sets or “clusters”, according to the degree of overlap between them. Each cluster was then given a unique identifying index, the molecular cluster index (MCI).

An MCI identifies a set of matched features on different images. Thus an MCI represents a protein or proteins eluting at equivalent positions in the 2D separation in different samples.

Construction of Profiles

After matching all component gels in the study to the final composite master image, the intensity of each feature was measured and stored. The end result of this analysis was the generation of a digital profile which contained, for each identified feature: 1) a unique identification code relative to corresponding feature within the composite master image (MCI), 2) the x, y coordinates of the features within the gel, 3) the isoelectric point (pI) of the features, 4) the apparent molecular weight (MW) of the features, 5) the signal value, 6) the standard deviation for each of the preceding measurements, and 7) a method of linking the MCI of each feature to the master gel to which this feature was matched. By virtue of a Laboratory Information Management System (LIMS), this MCI profile was traceable to the actual stored gel from which it was generated, so that proteins identified by computer analysis of gel profile databases could be retrieved. The LIMS also permitted the profile to be traced back to an original sample or patient.

Statistical Analysis of the Profiles

The MCI selection strategy is based on two overlapping statistical approaches:

(a) the use of the fold change. A fold change representing the ratio of the average normalized protein abundances of the EOFs within an MCI, was calculated for each MCI between each set of ErbB2-overexpressing cell line samples and the control. A cut-off of 2 for the fold change was used to select features.

(b) qualitative presence or absence. Only features present in 2 out of the 3 replicates for a sample were retained.

ERFs were present in all samples.

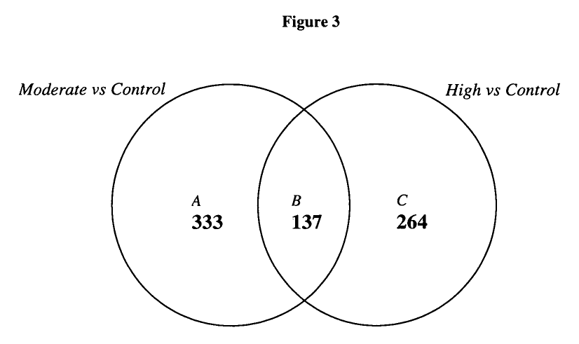

The EOFs were grouped into sets: those identified in moderately overexpressing ErbB2 cell lines (Venn position A), highly over expressing ErbB2 cell lines (Venn position C) and in both moderately and highly overexpressing ErbB2 cell lines (Venn position B) (refer to FIG. 3).

Recovery and Analysis of Selected Proteins

Proteins in EOFs were robotically excised and processed to generate tryptic digest peptides. Tryptic peptides were analyzed by mass spectrometry using a PerSeptive Biosystems Voyager-DETM STR Matrix-Assisted Laser Desorption Ionization Time-of-Flight (MALDI-TOF) mass spectrometer, and selected tryptic peptides were analyzed by tandem mass spectrometry (ErbB2 related cancer/ErbB2 related cancer) using a Micromass Quadrupole Time-of-Flight (Q-TOF) mass spectrometer (Micromass, Altrincham, U.K.) equipped with a nanoflow™ electrospray Z-spray source. For partial amino acid sequencing and identification of EOPIs uninterpreted tandem mass spectra of tryptic peptides were searched using the SEQUEST search program (Eng et al., 1994, J. Am. Soc. Mass Spectrom. 5:976-989), version v.C.1. Criteria for database identification included: the cleavage specificity of trypsin; the detection of a suite of a, b and y ions in peptides returned from the database. The database searched was database constructed of protein entries in the non-redundant database held by the National Centre for Biotechnology Information (NCBI) which is accessible at http://www.ncbi.nlm.nih.gov/. Following identification of proteins through spectral-spectral correlation using the SEQUEST program, masses detected in MALDI-TOF mass spectra were assigned to tryptic digest peptides within the proteins identified. In cases where no amino acid sequences could be identified through searching with uninterpreted ErbB2 related cancer/ErbB2 related cancer spectra of tryptic digest peptides using the SEQUEST program, tandem mass spectra of the peptides were interpreted manually, using methods known in the art. (In the case of interpretation of low-energy fragmentation mass spectra of peptide ions see Gaskell et al., 1992, Rapid Commun. Mass Spectrom. 6:658-662). The method described in PCT Application No. PCT/GB01/04034, which is incorporated herein by reference in its entirety, was also used to interpret mass spectra.

Results

These initial experiments identified 61 features that were decreased and 37 features that were increased in the cell line moderately overexpressing ErbB2 as compared with the control cell line, and 30 features that were decreased and 41 features that were increased in the cell line highly overexpressing ErbB2 as compared with the control cell line. Details of these EOFs are provided in Table I and defined in Lists I-IV. Each EOF was differentially present in one or more ErbB2-overexpressing cell line as compared with the control cell line. Partial amino acid sequences were determined for the differentially present EOPIs in these EOFs. Details of these EOPIs are provided in Table III and defined in Lists V-VIII.

Table VI details the differential expression of the EOFs/EOPIs identified in the ErbB2 overexpressing cell lines. The Preferred EOFs/EOPIs of the invention are EOF-86/EOPI-19, EOF-106/EOPI-22, EOF-163/EOPI-92, EOF-183/EOPI-95, EOF-201/EOPI-34, EOF-396/EOPI-115, EOF-483/EOPI-125, EOF-630/EOPI-59, EOF-634/EOPI-60, EOF-683/EOPI-62 and EOF-693/EOPI-63.

TABLE VI
Differential Expression of EOFs/EOPIs identified
in moderately (C3.6) and highly (C5.2)
overexpressing ErbB2 cell lines

| | | | | | Fold Change | |
|---|---|---|---|---|---|---|
| | | | | Venn | C3.6 | C5.2 |
| Feature | Isoform | | | Position | vs | vs |
| (EOF) | (EOPI) | pl | MW (Da) | (FIG. 3) | control | control |
| EOF-86 | EOPI-19 | 6.16 | 47,684 | A | −2.27 | |
| EOF-106 | EOPI-22 | 5.08 | 40,638 | A | −8.54 | |
| EOF-163 | EOPI-92 | 4.74 | 14,222 | A | 2.13 | |
| EOF-183 | EOPI-95 | 5.04 | 105,577 | A | −8.20 | |
| EOF-201 | EOPI-34 | 6.37 | 43,168 | A | −11.72 | |
| EOF-396 | EOPI-115 | 5.78 | 104,459 | B | 9.37 | 7.57 |
| EOF-483 | EOPI-125 | 5.39 | 127,995 | C | | 7.59 |
| EOF-630 | EOPI-59 | 4.98 | 21,851 | C | | 9.47 |
| EOF-634 | EOPI-60 | 5.05 | 16,573 | C | | 10.53 |
| EOF-683 | EOPI-62 | 5.45 | 13,968 | C | | 10.70 |
| EOF-693 | EOPI-63 | 4.99 | 10,445 | C | | 27.49 |
| EOF-34 | EOPI-67 | 6.29 | 113,362 | A | −3.31 | |
| EOF-35 | EOPI-1 | 6.51 | 110,562 | A | −3.37 | |
| EOF-44 | EOPI-2 | 6.44 | 85,990 | A | −2.13 | |
| EOF-48 | EOPI-3 | 4.93 | 80,273 | A | 2.26 | |
| EOF-51 | EOPI-4 | 6.61 | 74,208 | A | 2.39 | |
| EOF-57 | EOPI-70 | 5.16 | 68,895 | A | −7.22 | |
| EOF-61 | EOPI-71 | 7.73 | 61,037 | A | −2.13 | |
| EOF-63 | EOPI-5 | 5.02 | 58,032 | A | −5.36 | |
| EOF-66 | EOPI-72 | 5.47 | 56,144 | A | 4.69 | |
| EOF-67 | EOPI-6 | 6.92 | 56,452 | A | 2.29 | |
| EOF-70 | EOPI-7 | 4.80 | 54,791 | A | 3.48 | |
| EOF-70 | EOPI-8 | 4.80 | 54,791 | A | 3.48 | |
| EOF-72 | EOPI-9 | 5.58 | 52,905 | A | 5.08 | |
| EOF-73 | EOPI-11 | 4.76 | 53,395 | A | 4.31 | |
| EOF-74 | EOPI-12 | 5.27 | 52,894 | A | −10.45 | |
| EOF-75 | EOPI-13 | 5.16 | 51,876 | A | −3.28 | |
| EOF-76 | EOPI-14 | 4.83 | 52,196 | A | 4.85 | |
| EOF-76 | EOPI-15 | 4.83 | 52,196 | A | 4.85 | |
| EOF-80 | EOPI-16 | 4.67 | 51,143 | A | 2.62 | |
| EOF-81 | EOPI-17 | 5.17 | 49,168 | A | −3.02 | |
| EOF-81 | EOPI-18 | 5.17 | 49,168 | A | −3.02 | |
| EOF-83 | EOPI-73 | 5.06 | 49,351 | A | −2.02 | |
| EOF-84 | EOPI-74 | 4.77 | 49,233 | A | −6.51 | |
| EOF-87 | EOPI-75 | 4.61 | 47,205 | A | −4.23 | |
| EOF-90 | EOPI-20 | 5.39 | 44,908 | A | 3.58 | |
| EOF-94 | EOPI-77 | 6.13 | 44,019 | A | −2.44 | |
| EOF-94 | EOPI-78 | 6.13 | 44,019 | A | −2.44 | |
| EOF-99 | EOPI-80 | 5.40 | 42,809 | A | −15.60 | |
| EOF-102 | EOPI-21 | 5.39 | 42,227 | A | 3.39 | |
| EOF-104 | EOPI-81 | 6.72 | 41,786 | A | 2.05 | |
| EOF-107 | EOPI-23 | 5.74 | 40,619 | A | 2.49 | |
| EOF-110 | EOPI-24 | 6.16 | 39,933 | A | −2.40 | |
| EOF-111 | EOPI-82 | 4.77 | 40,050 | A | 2.87 | |
| EOF-120 | EOPI-84 | 5.77 | 36,187 | A | −8.96 | |
| EOF-129 | EOPI-85 | 4.61 | 31,848 | A | 6.19 | |
| EOF-133 | EOPI-86 | 5.16 | 31,009 | A | −2.20 | |
| EOF-135 | EOPI-87 | 5.60 | 30,301 | A | −4.69 | |
| EOF-136 | EOPI-25 | 5.14 | 29,942 | A | −3.00 | |
| EOF-137 | EOPI-26 | 5.39 | 29,939 | A | −2.94 | |
| EOF-138 | EOPI-88 | 6.10 | 29,850 | A | 3.02 | |
| EOF-139 | EOPI-89 | 5.21 | 29,877 | A | −3.79 | |
| EOF-145 | EOPI-90 | 5.04 | 28,469 | A | 2.51 | |
| EOF-149 | EOPI-27 | 5.54 | 23,266 | A | −19.31 | |
| EOF-153 | EOPI-91 | 5.45 | 21,277 | A | −2.40 | |
| EOF-156 | EOPI-28 | 5.06 | 19,901 | A | −2.12 | |
| EOF-166 | EOPI-29 | 4.96 | 12,052 | A | −5.07 | |
| EOF-167 | EOPI-94 | 5.26 | 12,062 | A | −3.21 | |
| EOF-182 | EOPI-30 | 5.10 | 106,575 | A | −2.00 | |
| EOF-188 | EOPI-96 | 6.29 | 69,345 | A | −4.77 | |
| EOF-196 | EOPI-31 | 5.55 | 50,803 | A | −2.10 | |
| EOF-196 | EOPI-32 | 5.55 | 50,803 | A | −2.10 | |
| EOF-197 | EOPI-33 | 5.59 | 50,859 | A | −9.09 | |
| EOF-206 | EOPI-97 | 4.83 | 33,251 | A | 2.05 | |
| EOF-225 | EOPI-98 | 4.78 | 12,156 | A | −6.55 | |
| EOF-242 | EOPI-35 | 5.28 | 75,945 | A | −5.12 | |
| EOF-251 | EOPI-99 | 5.63 | 39,810 | A | 13.85 | |
| EOF-254 | EOPI-100 | 5.77 | 30,205 | A | −9.07 | |
| EOF-263 | EOPI-102 | 6.45 | 107,728 | A | −3.98 | |
| EOF-267 | EOPI-104 | 5.52 | 55,278 | A | −4.23 | |
| EOF-342 | EOPI-105 | 6.10 | 127,560 | B | −3.93 | −5.15 |
| EOF-349 | EOPI-106 | 6.63 | 112,864 | B | 2.93 | 11.60 |
| EOF-350 | EOPI-107 | 6.35 | 110,931 | B | −3.69 | −3.34 |
| EOF-352 | EOPI-108 | 5.84 | 105,012 | B | 2.04 | 2.38 |
| EOF-361 | EOPI-36 | 5.45 | 53,541 | B | 4.77 | 3.57 |
| EOF-366 | EOPI-110 | 5.89 | 44,569 | B | 6.87 | 14.84 |
| EOF-370 | EOPI-37 | 4.73 | 38,321 | B | −2.30 | 3.05 |
| EOF-371 | EOPI-111 | 5.45 | 35,693 | B | 2.37 | 2.16 |
| EOF-371 | EOPI-112 | 5.45 | 35,693 | B | 2.37 | 2.16 |
| EOF-372 | EOPI-38 | 5.68 | 35,253 | B | −6.08 | 2.68 |
| EOF-376 | EOPI-39 | 5.40 | 28,445 | B | 2.38 | 2.67 |
| EOF-383 | EOPI-113 | 7.56 | 21,761 | B | −2.52 | −2.24 |
| EOF-387 | EOPI-40 | 4.57 | 13,644 | B | −2.12 | 2.10 |
| EOF-389 | EOPI-114 | 4.98 | 12,548 | B | 2.50 | 4.31 |
| EOF-400 | EOPI-116 | 5.10 | 63,380 | B | 8.44 | 5.50 |
| EOF-415 | EOPI-117 | 4.81 | 15,391 | B | −3.78 | −2.29 |
| EOF-419 | EOPI-118 | 5.96 | 10,109 | B | −28.42 | −11.36 |
| EOF-433 | EOPI-119 | 6.03 | 79,662 | B | −4.82 | −6.32 |
| EOF-435 | EOPI-121 | 6.90 | 78,821 | B | −3.44 | −4.51 |
| EOF-435 | EOPI-120 | 6.90 | 78,821 | B | −3.44 | −4.51 |
| EOF-444 | EOPI-122 | 5.89 | 42,782 | B | −13.52 | −17.72 |
| EOF-460 | EOPI-123 | 4.69 | 11,984 | B | −3.56 | −4.67 |
| EOF-482 | EOPI-124 | 6.04 | 128,802 | C | | 2.01 |
| EOF-489 | EOPI-126 | 5.00 | 120,241 | C | | −2.41 |
| EOF-490 | EOPI-41 | 6.55 | 111,664 | C | | −2.98 |
| EOF-493 | EOPI-42 | 5.08 | 107,091 | C | | −13.89 |
| EOF-501 | EOPI-127 | 5.46 | 77,552 | C | | 2.60 |
| EOF-510 | EOPI-43 | 6.16 | 62,248 | C | | 2.09 |
| EOF-513 | EOPI-129 | 6.93 | 59,686 | C | | 2.46 |
| EOF-523 | EOPI-44 | 4.95 | 53,870 | C | | 15.70 |
| EOF-526 | EOPI-45 | 6.79 | 51,978 | C | | −3.56 |
| EOF-527 | EOPI-46 | 5.84 | 50,081 | C | | 9.81 |
| EOF-532 | EOPI-130 | 6.69 | 47,218 | C | | −6.60 |
| EOF-533 | EOPI-47 | 6.99 | 45,426 | C | | 2.52 |
| EOF-534 | EOPI-48 | 5.29 | 44,549 | C | | 2.28 |
| EOF-536 | EOPI-131 | 6.61 | 42,351 | C | | 4.48 |
| EOF-544 | EOPI-49 | 4.66 | 35,091 | C | | −2.89 |
| EOF-546 | EOPI-50 | 4.66 | 34,343 | C | | −2.00 |
| EOF-550 | EOPI-133 | 6.73 | 31,505 | C | | 2.08 |
| EOF-552 | EOPI-51 | 5.59 | 30,717 | C | | −2.16 |
| EOF-553 | EOPI-134 | 5.76 | 30,422 | C | | −15.14 |
| EOF-554 | EOPI-136 | 5.34 | 29,967 | C | | −2.63 |
| EOF-556 | EOPI-137 | 5.61 | 27,964 | C | | 13.33 |
| EOF-560 | EOPI-139 | 7.57 | 25,483 | C | | 2.12 |
| EOF-562 | EOPI-140 | 5.61 | 23,876 | C | | 3.41 |
| EOF-574 | EOPI-52 | 4.65 | 18,637 | C | | 24.63 |
| EOF-578 | EOPI-53 | 4.83 | 16,715 | C | | 2.53 |
| EOF-582 | EOPI-54 | 4.53 | 14,162 | C | | 3.77 |
| EOF-582 | EOPI-55 | 4.53 | 14,162 | C | | 3.77 |
| EOF-584 | EOPI-141 | 5.55 | 12,556 | C | | −6.25 |
| EOF-609 | EOPI-56 | 5.54 | 52,543 | C | | 10.24 |
| EOF-611 | EOPI-57 | 4.84 | 48,949 | C | | −2.11 |
| EOF-612 | EOPI-58 | 4.91 | 48,777 | C | | −2.90 |
| EOF-636 | EOPI-143 | 4.52 | 15,011 | C | | −2.29 |
| EOF-665 | EOPI-61 | 5.60 | 35,566 | C | | 27.56 |
| EOF-711 | EOPI-144 | 5.09 | 29,751 | C | | −7.28 |
| EOF-713 | EOPI-64 | 4.93 | 27,497 | C | | −26.48 |
| EOF-721 | EOPI-65 | 5.28 | 11,456 | C | | −12.13 |
| EOF-724 | EOPI-145 | 5.79 | 42,413 | C | | −12.93 |

Additional EOFs that were identified using the reference protocol and are differentially expressed in ErbB2 related breast cancer are provided in Table VII below. These additional EOFs can be used in the present invention as described above in relation to the EOFs defined in Table I.

The present invention is not to be limited in terms of the particular embodiments described in this application, which are intended as single illustrations of individual aspects of the invention. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The contents of each reference, patent and patent application cited in this application is hereby incorporated by reference in its entirety.

TABLE VII
Additional Breast Cancer Associated Features (BFs)

| | | | | Fold | Fold | | | | | Fold | Fold |
|---|---|---|---|---|---|---|---|---|---|---|---|
| Feature | | | Venn | Change | Change | Feature | | | Venn | Change | Change |
| (EOF) | pl | MW (Da) | pos. | C36 | C52 | (EOF) | pl | MW (Da) | pos. | C36 | C52 |
| EOF-8 | 5.62 | 148,194 | A | −3.75 | | EOF-49 | 5.48 | 79,762 | A | −2.01 | |
| EOF-9 | 5.68 | 148,164 | A | −2.32 | | EOF-50 | 8.43 | 74,810 | A | −7.40 | |
| EOF-10 | 5.85 | 145,403 | A | −5.88 | | EOF-52 | 7.24 | 73,682 | A | −11.11 | |
| EOF-11 | 6.69 | 143,907 | A | −3.08 | | EOF-53 | 6.41 | 72,606 | A | −2.25 | |
| EOF-12 | 6.77 | 143,926 | A | −4.23 | | EOF-54 | 4.73 | 72,332 | A | −2.28 | |
| EOF-13 | 6.89 | 143,299 | A | −6.08 | | EOF-55 | 4.79 | 70,837 | A | −8.09 | |
| EOF-14 | 6.39 | 143,881 | A | −3.10 | | EOF-56 | 5.65 | 69,635 | A | −2.09 | |
| EOF-15 | 7.07 | 143,079 | A | −3.76 | | EOF-58 | 6.65 | 68,143 | A | −5.70 | |
| EOF-16 | 7.56 | 143,801 | A | −4.38 | | EOF-59 | 6.40 | 65,843 | A | −3.25 | |
| EOF-17 | 6.48 | 142,944 | A | −2.65 | | EOF-60 | 7.92 | 61,651 | A | −13.62 | |
| EOF-18 | 5.20 | 141,398 | A | −6.50 | | EOF-69 | 4.96 | 55,868 | A | −4.87 | |
| EOF-19 | 6.12 | 140,834 | A | −3.82 | | EOF-77 | 6.46 | 52,306 | A | −2.65 | |
| EOF-22 | 5.85 | 134,467 | A | −2.20 | | EOF-78 | 5.37 | 52,199 | A | −3.69 | |
| EOF-24 | 7.92 | 129,855 | A | −4.82 | | EOF-79 | 7.09 | 51,292 | A | −6.75 | |
| EOF-26 | 6.24 | 128,605 | A | −5.29 | | EOF-88 | 5.01 | 46,738 | A | −6.42 | |
| EOF-28 | 5.58 | 119,657 | A | −2.40 | | EOF-89 | 6.07 | 46,385 | A | −5.66 | |
| EOF-29 | 4.98 | 116,876 | A | −6.31 | | EOF-91 | 5.73 | 46,016 | A | −9.42 | |
| EOF-30 | 6.31 | 116,716 | A | −3.12 | | EOF-92 | 5.51 | 45,436 | A | −2.94 | |
| EOF-32 | 5.56 | 115,695 | A | −2.04 | | EOF-95 | 7.86 | 43,786 | A | −7.23 | |
| EOF-36 | 5.20 | 107,791 | A | −7.81 | | EOF-100 | 5.51 | 42,773 | A | −2.86 | |
| EOF-40 | 6.44 | 93,871 | A | −3.52 | | EOF-105 | 5.34 | 41,233 | A | −3.65 | |
| EOF-41 | 5.98 | 92,798 | A | −2.25 | | EOF-108 | 5.80 | 40,175 | A | −9.37 | |
| EOF-42 | 6.89 | 92,453 | A | −6.45 | | EOF-109 | 8.18 | 40,177 | A | −10.31 | |
| EOF-43 | 5.13 | 90,156 | A | −3.07 | | EOF-112 | 4.69 | 38,828 | A | −2.95 | |
| EOF-46 | 6.03 | 83,606 | A | −2.91 | | EOF-116 | 6.98 | 37,983 | A | −2.08 | |
| EOF-47 | 6.25 | 83,646 | A | −3.34 | | EOF-117 | 6.60 | 37,082 | A | −3.57 | |
| EOF-118 | 5.51 | 36,727 | A | −9.56 | | EOF-178 | 7.82 | 153,126 | A | −4.45 | |
| EOF-119 | 5.31 | 36,288 | A | −2.96 | | EOF-179 | 5.09 | 130,668 | A | −4.45 | |
| EOF-121 | 6.22 | 34,692 | A | −2.80 | | EOF-180 | 7.24 | 127,662 | A | −5.14 | |
| EOF-122 | 5.42 | 34,538 | A | −4.45 | | EOF-181 | 7.22 | 110,398 | A | −8.34 | |
| EOF-124 | 5.24 | 33,933 | A | −4.43 | | EOF-184 | 8.15 | 93,293 | A | −6.06 | |
| EOF-125 | 10.03 | 33,817 | A | −3.29 | | EOF-185 | 5.81 | 87,262 | A | −3.18 | |
| EOF-126 | 8.44 | 32,844 | A | −7.32 | | EOF-186 | 7.79 | 80,399 | A | −4.58 | |
| EOF-127 | 5.38 | 32,489 | A | −6.67 | | EOF-187 | 6.32 | 76,107 | A | −2.64 | |
| EOF-128 | 6.46 | 31,977 | A | −2.21 | | EOF-189 | 4.97 | 66,840 | A | −2.90 | |
| EOF-130 | 4.92 | 31,680 | A | −3.07 | | EOF-191 | 5.07 | 55,866 | A | −5.31 | |
| EOF-131 | 5.01 | 31,523 | A | −4.07 | | EOF-192 | 8.02 | 55,219 | A | −7.79 | |
| EOF-134 | 6.20 | 30,987 | A | −2.50 | | EOF-195 | 5.50 | 53,868 | A | −4.93 | |
| EOF-140 | 6.66 | 29,790 | A | −5.20 | | EOF-198 | 5.41 | 47,665 | A | −3.89 | |
| EOF-144 | 7.66 | 29,219 | A | −5.21 | | EOF-199 | 4.96 | 47,273 | A | −4.71 | |
| EOF-147 | 5.05 | 27,110 | A | −6.39 | | EOF-200 | 6.03 | 45,868 | A | −4.46 | |
| EOF-150 | 5.67 | 23,057 | A | −4.26 | | EOF-202 | 4.62 | 42,819 | A | −3.56 | |
| EOF-151 | 5.45 | 22,453 | A | −2.44 | | EOF-205 | 6.17 | 33,429 | A | −3.01 | |
| EOF-154 | 5.86 | 20,533 | A | −2.07 | | EOF-207 | 5.85 | 33,007 | A | −6.29 | |
| EOF-157 | 6.03 | 18,674 | A | −2.46 | | EOF-208 | 5.82 | 32,651 | A | −6.44 | |
| EOF-158 | 5.02 | 17,704 | A | −6.94 | | EOF-209 | 5.84 | 31,567 | A | −5.73 | |
| EOF-160 | 5.50 | 15,913 | A | −4.34 | | EOF-210 | 5.72 | 27,005 | A | −2.39 | |
| EOF-165 | 5.63 | 12,602 | A | −2.78 | | EOF-211 | 6.60 | 26,853 | A | −2.17 | |
| EOF-168 | 5.07 | 11,927 | A | −4.49 | | EOF-213 | 4.92 | 23,532 | A | −2.11 | |
| EOF-170 | 5.35 | 11,395 | A | −5.43 | | EOF-214 | 5.58 | 22,438 | A | −6.02 | |
| EOF-171 | 8.12 | 11,393 | A | −10.19 | | EOF-215 | 5.40 | 21,345 | A | −4.64 | |
| EOF-173 | 7.89 | 10,882 | A | −21.02 | | EOF-216 | 5.46 | 21,270 | A | −3.84 | |
| EOF-217 | 5.73 | 21,266 | A | −4.15 | | EOF-258 | 5.57 | 13,634 | A | −4.56 | |
| EOF-218 | 7.36 | 21,235 | A | −4.38 | | EOF-261 | 5.49 | 10,591 | A | −3.26 | |
| EOF-220 | 4.96 | 19,589 | A | −2.74 | | EOF-262 | 6.29 | 137,811 | A | −5.33 | |
| EOF-221 | 5.39 | 18,903 | A | −2.43 | | EOF-264 | 4.91 | 91,185 | A | −3.60 | |
| EOF-223 | 4.96 | 16,805 | A | −2.99 | | EOF-265 | 7.97 | 78,567 | A | −5.65 | |
| EOF-224 | 4.79 | 14,477 | A | −3.45 | | EOF-266 | 6.94 | 68,110 | A | −3.46 | |
| EOF-226 | 4.46 | 11,067 | A | −3.50 | | EOF-268 | 5.39 | 13,355 | A | −4.94 | |
| EOF-227 | 6.38 | 10,674 | A | −8.92 | | EOF-272 | 5.22 | 94,391 | A | −4.05 | |
| EOF-232 | 5.22 | 137,297 | A | −3.86 | | EOF-275 | 7.97 | 19,798 | A | −3.78 | |
| EOF-233 | 6.36 | 133,517 | A | −4.66 | | EOF-1 | 5.33 | 189,811 | A | 4.01 | |
| EOF-235 | 6.36 | 116,423 | A | −9.26 | | EOF-2 | 5.32 | 183,762 | A | 4.22 | |
| EOF-236 | 6.54 | 106,032 | A | −5.19 | | EOF-3 | 5.42 | 183,645 | A | 5.04 | |
| EOF-237 | 5.38 | 104,607 | A | −2.43 | | EOF-4 | 5.48 | 182,932 | A | 4.58 | |
| EOF-238 | 5.01 | 96,528 | A | −3.70 | | EOF-5 | 5.53 | 183,368 | A | 3.39 | |
| EOF-243 | 5.88 | 69,590 | A | −4.61 | | EOF-6 | 5.59 | 182,533 | A | 4.90 | |
| EOF-244 | 8.04 | 68,448 | A | −7.88 | | EOF-7 | 5.47 | 157,783 | A | 3.18 | |
| EOF-245 | 5.32 | 55,038 | A | −3.82 | | EOF-20 | 7.20 | 137,422 | A | 3.77 | |
| EOF-247 | 5.32 | 49,239 | A | −3.92 | | EOF-21 | 5.35 | 134,625 | A | 4.11 | |
| EOF-248 | 5.71 | 47,231 | A | −3.91 | | EOF-23 | 5.02 | 133,443 | A | 2.03 | |
| EOF-249 | 5.74 | 42,991 | A | −4.42 | | EOF-25 | 7.28 | 128,901 | A | 4.81 | |
| EOF-250 | 8.19 | 42,086 | A | −4.51 | | EOF-27 | 5.25 | 121,489 | A | 3.73 | |
| EOF-252 | 5.14 | 35,365 | A | −6.23 | | EOF-31 | 6.52 | 116,996 | A | 3.33 | |
| EOF-253 | 5.23 | 33,751 | A | −3.80 | | EOF-33 | 5.42 | 114,364 | A | 2.71 | |
| EOF-255 | 5.96 | 29,598 | A | −2.72 | | EOF-37 | 5.29 | 104,426 | A | 2.92 | |
| EOF-256 | 6.18 | 20,319 | A | −2.05 | | EOF-38 | 5.17 | 99,468 | A | 2.10 | |
| EOF-257 | 4.98 | 20,123 | A | −4.25 | | EOF-39 | 6.29 | 100,291 | A | 3.42 | |
| EOF-45 | 6.89 | 86,847 | A | 2.31 | | EOF-161 | 8.00 | 16,002 | A | 3.60 | |
| EOF-62 | 6.72 | 59,843 | A | 2.12 | | EOF-162 | 9.45 | 14,942 | A | 2.08 | |
| EOF-64 | 7.71 | 57,348 | A | 2.04 | | EOF-164 | 5.57 | 13,162 | A | 6.63 | |
| EOF-65 | 7.44 | 56,631 | A | 2.51 | | EOF-169 | 5.54 | 11,815 | A | 5.28 | |
| EOF-68 | 7.57 | 56,133 | A | 2.07 | | EOF-172 | 6.31 | 10,977 | A | 11.56 | |
| EOF-71 | 6.14 | 54,588 | A | 2.26 | | EOF-174 | 4.86 | 10,597 | A | 2.49 | |
| EOF-82 | 8.44 | 49,684 | A | 2.72 | | EOF-175 | 6.43 | 10,155 | A | 12.42 | |
| EOF-85 | 4.74 | 48,818 | A | 2.49 | | EOF-176 | 8.13 | 9,988 | A | 2.46 | |
| EOF-93 | 5.75 | 44,182 | A | 2.26 | | EOF-177 | 5.29 | 181,830 | A | 2.73 | |
| EOF-97 | 4.91 | 42,632 | A | 2.62 | | EOF-190 | 5.48 | 60,252 | A | 10.99 | |
| EOF-98 | 4.95 | 42,165 | A | 3.62 | | EOF-193 | 5.35 | 53,847 | A | 4.22 | |
| EOF-101 | 7.01 | 42,453 | A | 9.15 | | EOF-194 | 5.38 | 53,776 | A | 2.85 | |
| EOF-103 | 8.06 | 41,939 | A | 2.38 | | EOF-203 | 5.05 | 42,342 | A | 3.20 | |
| EOF-113 | 5.21 | 39,159 | A | 2.55 | | EOF-204 | 5.59 | 37,684 | A | 4.45 | |
| EOF-114 | 5.73 | 39,092 | A | 6.60 | | EOF-212 | 4.27 | 23,716 | A | 7.04 | |
| EOF-115 | 7.64 | 38,403 | A | 2.67 | | EOF-219 | 4.60 | 19,793 | A | 3.25 | |
| EOF-123 | 6.70 | 33,990 | A | 4.21 | | EOF-222 | 5.68 | 17,538 | A | 4.17 | |
| EOF-132 | 4.94 | 31,206 | A | 3.10 | | EOF-228 | 4.74 | 10,004 | A | 8.67 | |
| EOF-141 | 7.59 | 29,847 | A | 4.63 | | EOF-229 | 6.70 | 157,485 | A | 3.60 | |
| EOF-142 | 7.90 | 29,695 | A | 2.56 | | EOF-230 | 5.50 | 151,632 | A | 3.98 | |
| EOF-143 | 5.85 | 29,213 | A | 2.35 | | EOF-231 | 5.39 | 145,455 | A | 3.40 | |
| EOF-146 | 5.76 | 27,120 | A | 9.23 | | EOF-234 | 5.57 | 121,152 | A | 4.48 | |
| EOF-148 | 6.60 | 23,597 | A | 3.11 | | EOF-239 | 4.77 | 87,620 | A | 2.60 | |
| EOF-152 | 4.61 | 21,917 | A | 5.14 | | EOF-240 | 5.49 | 85,505 | A | 3.12 | |
| EOF-155 | 4.46 | 19,867 | A | 2.39 | | EOF-241 | 4.66 | 78,685 | A | 6.18 | |
| EOF-159 | 6.69 | 16,924 | A | 4.14 | | EOF-246 | 5.54 | 54,568 | A | 4.53 | |
| EOF-259 | 5.28 | 11,379 | A | 2.70 | | EOF-295 | 6.83 | 65,561 | A | 10.03 | |
| EOF-260 | 6.85 | 11,266 | A | 3.23 | | EOF-296 | 5.68 | 58,246 | A | 3.59 | |
| EOF-269 | 7.56 | 165,621 | A | 4.44 | | EOF-297 | 5.00 | 57,653 | A | 8.19 | |
| EOF-270 | 7.76 | 164,892 | A | 8.86 | | EOF-298 | 5.60 | 55,899 | A | 3.67 | |
| EOF-271 | 6.11 | 147,799 | A | 3.34 | | EOF-299 | 4.62 | 55,617 | A | 3.72 | |
| EOF-273 | 6.04 | 37,915 | A | 2.36 | | EOF-300 | 5.80 | 54,186 | A | 5.07 | |
| EOF-274 | 4.80 | 25,076 | A | 6.10 | | EOF-301 | 6.09 | 53,878 | A | 4.50 | |
| EOF-276 | 5.00 | 12,336 | A | 4.91 | | EOF-302 | 6.39 | 51,541 | A | 2.97 | |
| EOF-277 | 5.01 | 234,891 | A | 3.62 | | EOF-303 | 4.63 | 49,995 | A | 2.44 | |
| EOF-278 | 7.02 | 199,155 | A | 5.11 | | EOF-304 | 7.34 | 48,377 | A | 6.86 | |
| EOF-279 | 7.51 | 199,438 | A | 3.63 | | EOF-305 | 6.72 | 47,173 | A | 3.95 | |
| EOF-280 | 7.15 | 198,205 | A | 5.37 | | EOF-306 | 4.70 | 45,166 | A | 3.14 | |
| EOF-281 | 4.83 | 197,761 | A | 4.32 | | EOF-307 | 4.78 | 44,022 | A | 3.75 | |
| EOF-282 | 5.19 | 198,206 | A | 2.75 | | EOF-308 | 7.25 | 42,786 | A | 5.51 | |
| EOF-283 | 5.97 | 177,889 | A | 3.21 | | EOF-309 | 6.47 | 37,577 | A | 4.08 | |
| EOF-284 | 6.10 | 170,295 | A | 3.51 | | EOF-310 | 4.57 | 37,003 | A | 4.80 | |
| EOF-285 | 7.22 | 158,734 | A | 4.17 | | EOF-311 | 9.77 | 35,418 | A | 21.02 | |
| EOF-286 | 6.56 | 149,455 | A | 2.37 | | EOF-312 | 5.09 | 30,785 | A | 5.15 | |
| EOF-287 | 5.79 | 136,996 | A | 3.23 | | EOF-313 | 4.63 | 23,588 | A | 2.94 | |
| EOF-288 | 6.85 | 125,336 | A | 3.99 | | EOF-314 | 6.64 | 22,867 | A | 4.21 | |
| EOF-289 | 6.71 | 120,646 | A | 3.35 | | EOF-315 | 4.44 | 19,003 | A | 3.06 | |
| EOF-290 | 6.14 | 117,279 | A | 2.30 | | EOF-316 | 5.57 | 18,454 | A | 3.71 | |
| EOF-291 | 8.49 | 108,852 | A | 3.33 | | EOF-317 | 7.75 | 16,491 | A | 5.25 | |
| EOF-292 | 7.56 | 97,716 | A | 6.26 | | EOF-318 | 8.34 | 15,524 | A | 7.03 | |
| EOF-293 | 6.03 | 92,937 | A | 2.69 | | EOF-319 | 4.93 | 13,701 | A | 2.09 | |
| EOF-294 | 7.63 | 73,005 | A | 3.38 | | EOF-320 | 8.29 | 12,385 | A | 5.76 | |
| EOF-321 | 7.96 | 173,657 | A | 3.53 | | EOF-385 | 6.33 | 18,800 | B | −9.06 | −5.29 |
| EOF-322 | 7.39 | 171,279 | A | 4.15 | | EOF-386 | 5.10 | 14,959 | B | −3.98 | 2.30 |
| EOF-323 | 4.71 | 115,932 | A | 3.44 | | EOF-390 | 7.36 | 11,451 | B | −3.34 | −3.81 |
| EOF-324 | 4.87 | 111,421 | A | 2.35 | | EOF-392 | 5.23 | 10,375 | B | −4.50 | −5.89 |
| EOF-325 | 6.73 | 110,294 | A | 3.38 | | EOF-394 | 7.72 | 160,911 | B | −4.34 | −2.03 |
| EOF-326 | 7.77 | 75,646 | A | 4.83 | | EOF-409 | 4.99 | 30,617 | B | −6.69 | −3.03 |
| EOF-327 | 4.90 | 40,471 | A | 2.91 | | EOF-410 | 7.67 | 28,425 | B | −4.02 | −5.83 |
| EOF-328 | 9.33 | 34,800 | A | 5.23 | | EOF-417 | 5.55 | 10,555 | B | −2.67 | 2.64 |
| EOF-329 | 4.53 | 31,546 | A | 3.65 | | EOF-424 | 8.87 | 168,240 | B | −5.01 | −6.56 |
| EOF-330 | 5.46 | 20,479 | A | 12.04 | | EOF-425 | 5.13 | 154,290 | B | −6.29 | −8.24 |
| EOF-331 | 4.78 | 18,701 | A | 4.44 | | EOF-426 | 9.51 | 154,224 | B | −2.74 | −3.59 |
| EOF-332 | 5.73 | 17,815 | A | 3.28 | | EOF-427 | 5.79 | 125,222 | B | −4.43 | −5.81 |
| EOF-333 | 6.69 | 14,844 | A | 2.20 | | EOF-428 | 4.63 | 118,080 | B | −4.14 | −5.42 |
| EOF-340 | 4.84 | 136,957 | B | −2.72 | −3.57 | EOF-429 | 4.69 | 118,166 | B | −3.57 | −4.68 |
| EOF-341 | 8.59 | 136,962 | B | −2.98 | −14.56 | EOF-431 | 4.56 | 117,340 | B | −2.55 | −3.34 |
| EOF-345 | 6.57 | 120,150 | B | −6.14 | −8.05 | EOF-432 | 6.24 | 109,670 | B | −6.32 | −8.28 |
| EOF-348 | 7.58 | 112,993 | B | −2.10 | −2.45 | EOF-434 | 6.10 | 77,769 | B | −2.38 | −10.62 |
| EOF-351 | 7.55 | 107,953 | B | −3.53 | −4.63 | EOF-436 | 6.17 | 62,258 | B | −3.88 | −5.08 |
| EOF-353 | 5.34 | 104,067 | B | −2.19 | 2.03 | EOF-437 | 8.00 | 61,727 | B | −6.21 | −8.13 |
| EOF-360 | 8.21 | 54,623 | B | −4.91 | −6.43 | EOF-438 | 6.65 | 60,879 | B | −3.56 | −4.66 |
| EOF-365 | 7.16 | 46,025 | B | −2.70 | −2.06 | EOF-439 | 5.08 | 59,587 | B | −3.40 | −4.46 |
| EOF-368 | 7.34 | 39,618 | B | −3.20 | −2.21 | EOF-440 | 5.84 | 49,881 | B | −2.64 | −3.45 |
| EOF-373 | 5.31 | 33,898 | B | −14.76 | −3.19 | EOF-441 | 5.67 | 47,330 | B | −6.30 | −8.25 |
| EOF-375 | 8.04 | 28,782 | B | −11.50 | −15.06 | EOF-442 | 4.81 | 46,192 | B | −2.98 | −3.90 |
| EOF-378 | 6.38 | 25,179 | B | −3.01 | −2.22 | EOF-443 | 5.90 | 46,152 | B | −3.42 | −4.48 |
| EOF-379 | 5.27 | 23,406 | B | −2.63 | 2.71 | EOF-445 | 7.53 | 42,515 | B | −5.86 | −7.68 |
| EOF-446 | 7.25 | 35,271 | B | −15.61 | −20.46 | EOF-336 | 5.32 | 162,973 | B | 3.14 | 8.11 |
| EOF-447 | 6.27 | 34,467 | B | −2.53 | −3.32 | EOF-337 | 5.56 | 155,152 | B | 4.35 | 7.59 |
| EOF-448 | 5.69 | 28,166 | B | −4.17 | −5.46 | EOF-338 | 5.89 | 140,312 | B | 3.49 | 8.31 |
| EOF-449 | 5.27 | 26,393 | B | −2.81 | −3.68 | EOF-339 | 5.47 | 139,751 | B | 5.78 | 6.32 |
| EOF-450 | 6.10 | 23,569 | B | −4.26 | −5.58 | EOF-343 | 6.27 | 124,982 | B | 4.27 | 8.64 |
| EOF-451 | 5.78 | 23,395 | B | −2.38 | −3.11 | EOF-344 | 5.98 | 120,445 | B | 2.70 | 2.42 |
| EOF-452 | 6.71 | 22,347 | B | −3.54 | −4.64 | EOF-346 | 5.16 | 117,073 | B | 8.88 | 12.31 |
| EOF-453 | 4.74 | 20,554 | B | −2.49 | −3.26 | EOF-347 | 5.05 | 112,474 | B | 6.70 | 6.92 |
| EOF-454 | 8.06 | 16,788 | B | −4.90 | −6.42 | EOF-354 | 7.06 | 72,024 | B | 7.35 | 8.74 |
| EOF-455 | 8.28 | 16,806 | B | −2.59 | −39.38 | EOF-355 | 7.27 | 64,521 | B | 3.16 | 2.68 |
| EOF-456 | 5.65 | 15,309 | B | −2.64 | −3.46 | EOF-356 | 8.59 | 58,137 | B | 5.26 | 2.33 |
| EOF-457 | 6.25 | 13,851 | B | −10.80 | −14.15 | EOF-357 | 6.50 | 58,302 | B | 5.09 | 5.23 |
| EOF-458 | 4.70 | 13,225 | B | −2.01 | −2.63 | EOF-358 | 8.31 | 56,802 | B | 3.05 | 2.02 |
| EOF-459 | 5.78 | 12,770 | B | −3.54 | −4.64 | EOF-359 | 4.64 | 55,132 | B | 5.79 | 6.10 |
| EOF-461 | 4.37 | 10,421 | B | −2.85 | −3.74 | EOF-362 | 5.35 | 53,897 | B | 6.01 | 2.10 |
| EOF-462 | 5.62 | 166,050 | B | −7.49 | −9.81 | EOF-363 | 7.18 | 47,816 | B | 8.35 | 8.82 |
| EOF-463 | 4.68 | 86,524 | B | −2.48 | −3.25 | EOF-364 | 6.88 | 47,437 | B | 3.77 | 2.61 |
| EOF-464 | 5.58 | 39,386 | B | −3.57 | −4.68 | EOF-367 | 4.98 | 44,222 | B | 7.32 | 14.10 |
| EOF-465 | 8.21 | 21,537 | B | −4.32 | −5.66 | EOF-369 | 7.42 | 38,986 | B | 2.40 | 3.02 |
| EOF-466 | 7.74 | 20,946 | B | −3.96 | −5.19 | EOF-374 | 7.91 | 33,754 | B | 4.60 | 5.59 |
| EOF-467 | 5.32 | 20,812 | B | −3.93 | −5.15 | EOF-377 | 5.08 | 25,411 | B | 12.02 | 18.04 |
| EOF-468 | 6.04 | 19,079 | B | −2.93 | −3.84 | EOF-380 | 7.25 | 22,880 | B | 3.49 | 5.97 |
| EOF-469 | 4.48 | 18,821 | B | −5.52 | −7.23 | EOF-381 | 5.40 | 22,590 | B | 4.67 | 4.14 |
| EOF-470 | 5.45 | 11,537 | B | −2.51 | −3.29 | EOF-382 | 7.44 | 22,122 | B | 3.77 | 6.17 |
| EOF-334 | 5.48 | 171,273 | B | 2.67 | 4.48 | EOF-384 | 4.53 | 21,676 | B | 5.86 | 4.21 |
| EOF-335 | 5.59 | 171,079 | B | 7.91 | 5.93 | EOF-388 | 5.41 | 13,274 | B | 2.54 | 5.33 |
| EOF-391 | 6.66 | 10,768 | B | 2.88 | 4.93 | EOF-479 | 7.64 | 148,749 | C | | −5.50 |
| EOF-393 | 6.33 | 163,902 | B | 2.84 | 4.01 | EOF-480 | 5.31 | 147,477 | C | | −3.97 |
| EOF-395 | 8.26 | 123,441 | B | 3.50 | 3.94 | EOF-481 | 6.52 | 143,417 | C | | −4.19 |
| EOF-397 | 6.16 | 97,316 | B | 3.64 | 5.71 | EOF-487 | 5.03 | 124,308 | C | | −5.47 |
| EOF-398 | 5.14 | 78,124 | B | 6.30 | 5.73 | EOF-495 | 7.06 | 87,634 | C | | −7.32 |
| EOF-399 | 4.71 | 74,498 | B | 2.64 | 4.02 | EOF-496 | 7.67 | 86,665 | C | | −2.20 |
| EOF-401 | 5.56 | 62,397 | B | 5.22 | 5.84 | EOF-497 | 5.51 | 85,388 | C | | −2.13 |
| EOF-402 | 4.84 | 54,058 | B | 5.52 | 7.38 | EOF-499 | 5.56 | 80,369 | C | | −13.65 |
| EOF-403 | 4.71 | 53,567 | B | 2.60 | 4.33 | EOF-505 | 6.78 | 68,228 | C | | −5.67 |
| EOF-404 | 4.70 | 42,749 | B | 8.25 | 3.16 | EOF-506 | 8.08 | 66,619 | C | | −2.59 |
| EOF-405 | 4.86 | 35,115 | B | 3.12 | 3.54 | EOF-509 | 5.65 | 62,731 | C | | −2.34 |
| EOF-406 | 5.30 | 32,809 | B | 3.31 | 3.70 | EOF-514 | 4.61 | 58,141 | C | | −3.36 |
| EOF-407 | 4.87 | 30,831 | B | 4.02 | 3.64 | EOF-515 | 5.11 | 57,856 | C | | −2.09 |
| EOF-408 | 4.49 | 30,656 | B | 5.60 | 13.85 | EOF-520 | 7.93 | 56,475 | C | | −16.33 |
| EOF-411 | 5.46 | 24,213 | B | 11.39 | 8.21 | EOF-522 | 7.41 | 55,021 | C | | −8.68 |
| EOF-412 | 8.00 | 22,010 | B | 6.39 | 15.15 | EOF-524 | 7.69 | 53,024 | C | | −3.64 |
| EOF-413 | 4.78 | 20,692 | B | 4.16 | 3.98 | EOF-529 | 7.73 | 49,502 | C | | −18.30 |
| EOF-414 | 5.28 | 19,151 | B | 4.00 | 2.21 | EOF-537 | 4.65 | 37,429 | C | | −2.36 |
| EOF-416 | 4.46 | 13,584 | B | 8.06 | 5.03 | EOF-543 | 4.79 | 35,147 | C | | −2.06 |
| EOF-418 | 7.70 | 10,160 | B | 13.35 | 9.48 | EOF-545 | 6.02 | 34,545 | C | | −5.96 |
| EOF-420 | 6.58 | 144,769 | B | 2.18 | 3.21 | EOF-548 | 8.43 | 32,250 | C | | −6.71 |
| EOF-421 | 6.69 | 67,682 | B | 3.76 | 4.98 | EOF-551 | 7.49 | 31,412 | C | | −2.09 |
| EOF-422 | 6.37 | 60,600 | B | 4.80 | 12.90 | EOF-557 | 5.49 | 26,295 | C | | −10.36 |
| EOF-423 | 5.78 | 12,166 | B | 4.15 | 2.97 | EOF-558 | 4.84 | 26,636 | C | | −9.90 |
| EOF-430 | 4.52 | 117,430 | B | 2.12 | −2.64 | EOF-559 | 5.28 | 25,980 | C | | −2.57 |
| EOF-474 | 5.59 | 165,319 | C | | −5.13 | EOF-563 | 5.40 | 23,412 | C | | −3.11 |
| EOF-564 | 8.18 | 22,387 | C | | −2.46 | EOF-703 | 4.69 | 47,873 | C | | −5.37 |
| EOF-566 | 7.15 | 21,968 | C | | −3.11 | EOF-704 | 5.31 | 41,309 | C | | −5.54 |
| EOF-567 | 8.38 | 21,029 | C | | −10.93 | EOF-705 | 4.78 | 40,514 | C | | −4.32 |
| EOF-570 | 7.69 | 20,462 | C | | −7.43 | EOF-706 | 5.56 | 39,298 | C | | −4.61 |
| EOF-575 | 5.98 | 17,794 | C | | −2.20 | EOF-707 | 6.73 | 38,606 | C | | −11.16 |
| EOF-577 | 6.00 | 16,814 | C | | −2.07 | EOF-708 | 8.18 | 35,758 | C | | −13.43 |
| EOF-580 | 7.67 | 15,914 | C | | −2.15 | EOF-709 | 5.25 | 35,655 | C | | −5.96 |
| EOF-581 | 4.86 | 15,586 | C | | −5.69 | EOF-710 | 8.36 | 32,967 | C | | −8.55 |
| EOF-585 | 7.41 | 12,510 | C | | −2.18 | EOF-712 | 4.59 | 29,662 | C | | −3.81 |
| EOF-587 | 7.03 | 11,274 | C | | −22.76 | EOF-714 | 4.83 | 26,159 | C | | −2.43 |
| EOF-607 | 6.99 | 58,864 | C | | −2.36 | EOF-715 | 5.38 | 26,161 | C | | −5.28 |
| EOF-617 | 4.75 | 44,325 | C | | −2.42 | EOF-716 | 6.97 | 21,448 | C | | −4.51 |
| EOF-623 | 6.17 | 36,793 | C | | −2.74 | EOF-717 | 5.40 | 17,739 | C | | −2.61 |
| EOF-624 | 4.79 | 32,446 | C | | −2.11 | EOF-718 | 6.09 | 17,643 | C | | −2.61 |
| EOF-629 | 7.69 | 23,104 | C | | −2.57 | EOF-719 | 8.17 | 15,664 | C | | −20.15 |
| EOF-639 | 5.82 | 10,824 | C | | −3.05 | EOF-720 | 4.53 | 11,548 | C | | −2.58 |
| EOF-660 | 5.59 | 44,806 | C | | −3.36 | EOF-722 | 5.81 | 11,458 | C | | −10.37 |
| EOF-663 | 5.25 | 36,848 | C | | −2.16 | EOF-723 | 7.39 | 52,920 | C | | −6.80 |
| EOF-695 | 5.91 | 149,833 | C | | −4.32 | EOF-725 | 5.64 | 33,336 | C | | −10.97 |
| EOF-696 | 8.18 | 118,065 | C | | −3.28 | EOF-726 | 7.74 | 18,808 | C | | −9.98 |
| EOF-697 | 6.96 | 83,474 | C | | −6.06 | EOF-727 | 4.76 | 17,772 | C | | −19.22 |
| EOF-698 | 7.13 | 83,448 | C | | −4.56 | EOF-728 | 6.88 | 13,025 | C | | −4.30 |
| EOF-699 | 7.61 | 75,523 | C | | −9.41 | EOF-729 | 8.72 | 163,647 | C | | −3.12 |
| EOF-700 | 5.12 | 72,202 | C | | −6.97 | EOF-730 | 8.27 | 38,363 | C | | −10.03 |
| EOF-701 | 5.92 | 57,949 | C | | −6.98 | EOF-731 | 5.67 | 38,259 | C | | −7.83 |
| EOF-702 | 8.19 | 53,809 | C | | −12.22 | EOF-732 | 5.51 | 33,328 | C | | −17.23 |
| EOF-733 | 4.38 | 16,562 | C | | −14.94 | EOF-517 | 6.65 | 57,226 | C | | 6.57 |
| EOF-734 | 10.18 | 35,623 | C | | −22.44 | EOF-518 | 4.56 | 56,658 | C | | 2.06 |
| EOF-471 | 6.37 | 167,542 | C | | 3.54 | EOF-519 | 5.50 | 56,639 | C | | 2.51 |
| EOF-472 | 5.41 | 167,347 | C | | 2.30 | EOF-521 | 7.07 | 55,488 | C | | 2.65 |
| EOF-473 | 5.47 | 166,802 | C | | 5.18 | EOF-525 | 8.44 | 52,109 | C | | 2.41 |
| EOF-475 | 4.94 | 164,025 | C | | 3.35 | EOF-528 | 8.30 | 49,845 | C | | 3.77 |
| EOF-476 | 5.70 | 162,101 | C | | 4.61 | EOF-530 | 4.80 | 49,067 | C | | 2.91 |
| EOF-477 | 7.73 | 157,501 | C | | 5.49 | EOF-531 | 5.95 | 48,479 | C | | 8.45 |
| EOF-478 | 5.31 | 150,834 | C | | 4.45 | EOF-535 | 6.30 | 44,724 | C | | 4.52 |
| EOF-484 | 6.62 | 128,618 | C | | 2.22 | EOF-538 | 5.13 | 37,042 | C | | 7.41 |
| EOF-485 | 6.60 | 128,420 | C | | 3.70 | EOF-539 | 7.06 | 35,932 | C | | 6.40 |
| EOF-486 | 7.21 | 125,457 | C | | 5.03 | EOF-540 | 4.81 | 35,826 | C | | 9.38 |
| EOF-488 | 7.73 | 123,145 | C | | 2.40 | EOF-541 | 7.23 | 35,422 | C | | 2.09 |
| EOF-491 | 6.71 | 111,153 | C | | 2.74 | EOF-542 | 5.31 | 35,056 | C | | 2.06 |
| EOF-492 | 5.69 | 109,421 | C | | 2.03 | EOF-547 | 9.03 | 33,861 | C | | 2.04 |
| EOF-494 | 6.18 | 104,609 | C | | 7.42 | EOF-549 | 4.44 | 31,612 | C | | 3.70 |
| EOF-498 | 7.16 | 84,396 | C | | 2.47 | EOF-555 | 4.65 | 28,921 | C | | 3.18 |
| EOF-500 | 5.91 | 79,080 | C | | 2.26 | EOF-561 | 4.71 | 25,591 | C | | 16.01 |
| EOF-502 | 4.87 | 73,298 | C | | 2.04 | EOF-565 | 9.25 | 22,423 | C | | 2.22 |
| EOF-503 | 5.65 | 72,371 | C | | 2.05 | EOF-568 | 5.17 | 20,659 | C | | 2.59 |
| EOF-504 | 4.92 | 67,954 | C | | 3.44 | EOF-569 | 5.74 | 20,556 | C | | 7.96 |
| EOF-507 | 5.82 | 66,340 | C | | 2.41 | EOF-571 | 6.02 | 19,785 | C | | 2.24 |
| EOF-508 | 4.88 | 63,709 | C | | 13.41 | EOF-572 | 4.74 | 19,225 | C | | 12.09 |
| EOF-511 | 4.74 | 61,404 | C | | 2.72 | EOF-573 | 7.68 | 19,215 | C | | 13.89 |
| EOF-512 | 8.19 | 60,759 | C | | 13.84 | EOF-576 | 6.14 | 17,919 | C | | 5.84 |
| EOF-516 | 5.63 | 57,668 | C | | 3.58 | EOF-579 | 7.85 | 16,055 | C | | 4.91 |
| EOF-583 | 5.16 | 13,656 | C | | 2.96 | EOF-616 | 7.17 | 45,134 | C | | 9.98 |
| EOF-586 | 5.45 | 11,474 | C | | 2.14 | EOF-618 | 4.62 | 44,006 | C | | 2.01 |
| EOF-588 | 5.73 | 174,041 | C | | 3.95 | EOF-619 | 6.25 | 42,475 | C | | 4.39 |
| EOF-589 | 5.10 | 170,767 | C | | 4.50 | EOF-620 | 5.63 | 39,277 | C | | 3.88 |
| EOF-590 | 5.98 | 165,614 | C | | 2.30 | EOF-621 | 4.52 | 39,118 | C | | 10.40 |
| EOF-591 | 8.51 | 161,440 | C | | 5.86 | EOF-622 | 4.79 | 38,679 | C | | 6.49 |
| EOF-592 | 8.32 | 160,730 | C | | 8.09 | EOF-625 | 4.82 | 31,050 | C | | 3.58 |
| EOF-593 | 6.18 | 154,023 | C | | 4.23 | EOF-626 | 6.25 | 30,078 | C | | 2.68 |
| EOF-594 | 4.74 | 148,603 | C | | 2.28 | EOF-627 | 5.75 | 25,001 | C | | 2.69 |
| EOF-595 | 5.83 | 148,825 | C | | 2.80 | EOF-628 | 8.27 | 24,331 | C | | 5.36 |
| EOF-596 | 5.87 | 142,423 | C | | 4.20 | EOF-631 | 4.74 | 20,707 | C | | 2.16 |
| EOF-597 | 5.04 | 135,935 | C | | 5.62 | EOF-632 | 4.42 | 18,493 | C | | 4.43 |
| EOF-598 | 7.25 | 135,394 | C | | 4.32 | EOF-633 | 10.41 | 16,947 | C | | 16.49 |
| EOF-599 | 6.39 | 132,325 | C | | 5.91 | EOF-635 | 6.83 | 15,377 | C | | 4.29 |
| EOF-600 | 5.77 | 124,965 | C | | 4.12 | EOF-637 | 4.22 | 14,555 | C | | 12.12 |
| EOF-601 | 5.87 | 120,316 | C | | 4.54 | EOF-638 | 7.78 | 11,922 | C | | 10.50 |
| EOF-602 | 5.99 | 90,207 | C | | 2.59 | EOF-640 | 8.44 | 10,440 | C | | 5.83 |
| EOF-603 | 6.62 | 85,934 | C | | 7.10 | EOF-641 | 7.26 | 10,283 | C | | 29.59 |
| EOF-604 | 5.73 | 76,793 | C | | 7.45 | EOF-642 | 5.65 | 151,947 | C | | 3.16 |
| EOF-605 | 8.37 | 76,688 | C | | 12.56 | EOF-643 | 5.90 | 148,808 | C | | 3.45 |
| EOF-606 | 5.78 | 62,288 | C | | 3.56 | EOF-644 | 5.78 | 135,161 | C | | 10.56 |
| EOF-608 | 5.68 | 58,702 | C | | 3.86 | EOF-645 | 7.78 | 109,634 | C | | 4.77 |
| EOF-610 | 8.25 | 50,747 | C | | 2.36 | EOF-646 | 5.78 | 109,422 | C | | 6.35 |
| EOF-613 | 4.51 | 46,980 | C | | 4.40 | EOF-647 | 8.19 | 108,919 | C | | 6.32 |
| EOF-614 | 5.49 | 45,957 | C | | 4.74 | EOF-648 | 7.03 | 108,924 | C | | 4.08 |
| EOF-615 | 4.90 | 45,745 | C | | 5.72 | EOF-649 | 8.30 | 101,449 | C | | 4.87 |
| EOF-650 | 5.39 | 100,463 | C | | 3.12 | EOF-679 | 5.91 | 19,285 | C | | 4.10 |
| EOF-651 | 5.26 | 86,443 | C | | 4.69 | EOF-680 | 4.86 | 17,526 | C | | 2.15 |
| EOF-652 | 6.00 | 65,660 | C | | 8.35 | EOF-681 | 4.43 | 15,962 | C | | 3.17 |
| EOF-653 | 5.51 | 63,035 | C | | 4.91 | EOF-682 | 4.64 | 15,425 | C | | 6.20 |
| EOF-654 | 4.81 | 61,287 | C | | 8.99 | EOF-684 | 7.14 | 13,297 | C | | 2.98 |
| EOF-655 | 4.77 | 54,517 | C | | 4.86 | EOF-685 | 6.95 | 12,810 | C | | 4.76 |
| EOF-656 | 6.03 | 54,127 | C | | 3.73 | EOF-686 | 7.05 | 12,360 | C | | 2.72 |
| EOF-657 | 5.79 | 49,227 | C | | 4.82 | EOF-687 | 5.88 | 10,984 | C | | 15.34 |
| EOF-658 | 6.05 | 48,960 | C | | 3.68 | EOF-688 | 7.39 | 10,849 | C | | 5.30 |
| EOF-659 | 6.35 | 45,173 | C | | 7.07 | EOF-689 | 4.83 | 10,739 | C | | 8.25 |
| EOF-661 | 4.64 | 43,245 | C | | 3.10 | EOF-690 | 5.05 | 10,636 | C | | 8.98 |
| EOF-662 | 5.40 | 39,749 | C | | 3.79 | EOF-691 | 7.00 | 10,549 | C | | 28.75 |
| EOF-664 | 5.24 | 36,102 | C | | 3.07 | EOF-692 | 6.84 | 10,643 | C | | 7.23 |
| EOF-666 | 6.25 | 33,519 | C | | 3.62 | EOF-694 | 7.66 | 10,284 | C | | 6.01 |
| EOF-667 | 4.89 | 33,527 | C | | 3.38 | | | | | | |
| EOF-668 | 5.33 | 32,569 | C | | 4.10 | | | | | | |
| EOF-669 | 4.72 | 30,706 | C | | 4.19 | | | | | | |
| EOF-670 | 5.96 | 30,068 | C | | 5.79 | | | | | | |
| EOF-671 | 6.30 | 29,253 | C | | 20.07 | | | | | | |
| EOF-672 | 6.07 | 25,289 | C | | 5.43 | | | | | | |
| EOF-673 | 4.98 | 23,330 | C | | 7.71 | | | | | | |
| EOF-674 | 5.78 | 21,248 | C | | 3.06 | | | | | | |
| EOF-675 | 5.61 | 20,955 | C | | 12.31 | | | | | | |
| EOF-676 | 6.23 | 20,194 | C | | 4.22 | | | | | | |
| EOF-677 | 4.66 | 19,489 | C | | 6.21 | | | | | | |
| EOF-678 | 5.47 | 19,346 | C | | 13.06 | | | | | | |

We claim:

1. A method for screening or diagnosis of ErbB2 related cancer in a subject, for determining the stage or severity of ErbB2 related cancer in a subject, for identifying a subject at risk of developing ErbB2 related cancer, or for monitoring the effect of therapy administered to a subject having ErbB2 related cancer, said method comprising:
(a) analysing a test sample of body fluid from the subject by two-dimensional electrophoresis to generate a two-dimensional array of features, said array comprising one or more of the ErbB2 Overexpression Features (EOFs) as defined in Table I; and
(b) comparing the abundance of the one or more EOFs in the test sample with the abundance of the one or more EOFs in a tissue or body fluid sample from one or more subjects free from ErbB2 related cancer, or with a previously determined reference range for that feature in subjects free from ErbB2 related cancer, or with the abundance at least one Expression Reference Feature (ERF) in the test sample.

2. The method according to claim 1 wherein the EOF is selected from one or more of the following EOFs: EOF-86, EOF-106, EOF-163, EOF-183, EOF-201, EOF-396, EOF-483, EOF-630, EOF-634, EOF-683 or EOF-693.

3. A method for screening or diagnosis of ErbB2 related cancer in a subject, for determining the stage or severity of ErbB2 related cancer in a subject, for identifying a subject at risk of developing ErbB2 related cancer, or for monitoring the effect of therapy administered to a subject having ErbB2 related cancer, said method comprising quantitatively detecting, in a test sample of body fluid from the subject, one or more of the ErbB2 Overexpression Protein Isoforms (EOPIs) as defined in Table III.

4. The method according to claim 3 wherein the EOPI is selected from one or more of the following EOPIs: EOPI-19, EOPI-22, EOPI-34, EOPI-59, EOPI-60, EOPI-62, EOPI-63, EOPI-92, EOPI-95, EOPI-115 or EOPI-125.

5. The method according to claim 1 where the body fluid is blood, serum, plasma or urine.

6. The method according to claim 3 where the abundance of the one or more EOPIs in the test sample is compared with the abundance of the one ore more EOPIs in a sample from one or more subjects free from ErbB2 related cancer, or with a previously determined reference range for that feature in subjects free from ErbB2 related cancer, or with the abundance at least one Expression Reference Feature (ERF) in the test sample.

7. The method according to claim 3, wherein the step of quantitatively detecting comprises testing at least one aliquot of the first sample, said step of testing comprising:
(a) contacting the aliquot with an antibody that is immunospecific for a EOPI;
(b) quantitatively measuring the binding of the antibody and the EOPI; and
(c) comparing the results of step (b) with a predetermined reference range.

8. The method according to claim 7, wherein the step of quantitatively detecting comprises testing a plurality of aliquots with a plurality of antibodies cognate for a plurality of preselected EOPIs.

9. A pharmaceutical composition comprising an ErbB2 Overexpression Protein Isoform (EOPI) as defined in claim 3, or a nucleic acid encoding an EOPI, and a pharmaceutically acceptable carrier.

10. The pharmaceutical composition according to claim 9, wherein the ErbB2 Overexpression Protein Isoform (EOPI) is in recombinant form.

11. An antibody capable of immunospecific binding to an ErbB2 Overexpression Protein Isoform (EOPI) as defined in claim 3.

12. The method according to claim 7, wherein the antibody is a monoclonal antibody.

13. The method according to claim 7, wherein the antibody is selected from a chimeric antibody, a bispecific antibody and a humanised antibody.

14. The method according to claim 7, wherein the antibody binds to the EOPI with greater affinity than to another isoform of the EOPI.

15. A kit comprising one or more antibodies as claimed in claim 11 and/or one or more EOPIs as defined in claim 3, other reagents and instructions for use.

16. The kit of claim 15 for use in the screening or diagnosis of ErbB2 related cancer in a subject, for determining the stage or severity of ErbB2 related cancer in a subject, for identifying a subject at risk of developing ErbB2 related cancer, or for monitoring the effect of therapy administered to a subject having ErbB2 related cancer.

17. The kit according to claim 15 comprising a plurality of antibodies as claimed in claim 11 and/or a plurality of EOPIs as defined in claim 3.

18. A pharmaceutical composition comprising a therapeutically effective amount of an antibody, or a fragment or derivative of an antibody according to claim 11 and a pharmaceutically acceptable carrier.

19. A method of treating or preventing ErbB2 related cancer comprising administering to a subject in need of such treatment a therapeutically effective amount of an antibody as claimed in claim 11.

20. A method of treating or preventing ErbB2 related cancer comprising administering to a subject in need of such treatment or prevention a therapeutically effective amount of one or more of the ErbB2 Overexpression Protein Isoforms (EOPIs) as defined in claim 3 and/or a nucleic acid encoding said EOPIs.

21. A method of treating or preventing ErbB2 related cancer comprising administering to a subject in need of such treatment or prevention a therapeutically effective amount of a nucleic acid that inhibits the function of one or more of the ErbB2 Overexpression Protein Isoforms (EOPIs) as defined in claim 3.

22. The method according to claim 21, wherein the nucleic acid is a EOPI antisense nucleic acid or ribozyme.

23. A method of screening for agents that interact with one or more ErbB2 Overexpression Protein Isoforms (EOPIs) as defined in claim 3, fragments of EOPIs (EOPI fragment), polypeptides related to EOPIs (EOPI-related polypeptide), or EOPI-fusion proteins said method comprising:
(a) contacting an EOPI, an EOPI fragment, an EOPI-related polypeptide, or an EOPI-fusion protein with a candidate agent; and
(b) determining whether or not the candidate agent interacts with the EOPI, the EOPI fragment, the EOPI-related polypeptide, or the EOPI-fusion protein.

24. The method according to claim 23, wherein the determination of interaction between the candidate agent and the EOPI, EOPI fragment, EOPI-related polypeptide or EOPI-fusion protein comprises quantitatively detecting binding of the candidate agent and the EOPI, EOPI fragment, EOPI-related polypeptide or EOPI-fusion protein.

25. A method of screening for or identifying agents that modulate the expression or activity of one or more ErbB2 Overexpression Protein Isoforms (EOPIs) as defined in claim 3, fragments of EOPI (EOPI fragment), polypeptides related to EOPIs (EOPI-related polypeptide) or EOPI-fusion proteins comprising:
(a) contacting a first population of cells expressing the EOPI, EOPI fragment, EOPI-related polypeptide, or EOPI-fusion protein with a candidate agent;
(b) contacting a second population of cells expressing said EOPI, EOPI fragment, EOPI-related polypeptide, or EOPI-fusion protein with a control agent; and
(c) comparing the level of said EOPI, EOPI fragment, EOPI-related polypeptide, or EOPI-fusion protein or mRNA encoding said EOPI, EOPI fragment, EOPI-related polypeptide, or EOPI-fusion protein in the first and second populations of cells, or comparing the level of induction of a downstream effector in the first and second populations of cells.

26. A method of screening for or identifying agents that modulate the expression or activity of one or more ErbB2 Overexpression Protein Isoforms (EOPIs) as defined in claim 3, fragments of EOPIs (EOPI fragment), polypeptides related to EOPIs (EOPI-related polypeptide) or EOPI-fusion proteins said method comprising:
(a) administering a candidate agent to a first mammal or group of mammals;
(b) administering a control agent to a second mammal or group of mammals; and
(c) comparing the level of expression of the EOPI, EOPI fragment, EOPI-related polypeptide or EOPI-fusion protein, or mRNA encoding said EOPI, EOPI fragment, EOPI-related polypeptide or EOPI-fusion protein in the first and second groups, or comparing the level of induction of a downstream effector in the first and second groups.

27. The method as claimed in claim 26, wherein the mammals are animal models for ErbB2 related cancer.

28. The method according to claim 25, wherein administration of a candidate agent results in an increase in the level of said EOPI, EOPI fragment, EOPI-related polypeptide or EOPI-fusion protein, or mRNA encoding said EOPI, EOPI fragment, EOPI-related polypeptide, or EOPI-fusion protein, or said downstream effector in the first population of cells or mammals compared to the second population of cells or mammals.

29. The method according to claim 25, wherein administration of a candidate agent results in a decrease in the level of said EOPI, EOPI fragment, EOPI-related polypeptide, or EOPI-fusion protein, or mRNA encoding said EOPI, EOPI fragment, EOPI-related polypeptide, or EOPI-fusion protein, or said downstream effector in the first population of cells or mammals compared to the second population of cells or mammals.

30. The method as claimed in claim 25, wherein the levels of said EOPI, EOPI fragment, EOPI-related polypeptide, or EOPI-fusion protein, or mRNA encoding said EOPI, EOPI fragment, EOPI-related polypeptide, or EOPI-fusion protein, or of said downstream effector in the first and second groups are further compared to the level of said EOPI, EOPI fragment, EOPI-related polypeptide or EOPI-fusion protein, or mRNA encoding said EOPI, EOPI fragment, EOPI-related polypeptide or EOPI-fusion protein in normal control mammals.

31. The method according to claim 30, wherein said mammals are human subjects with ErbB2 related cancer.

32. A method of screening for or identifying agents that modulate the activity of one or more of the ErbB2 Overexpression Proteins Isoforms (EOPIs) as defined in claim 3, fragments of EOPIs (EOPI-fragment), polypeptides related to EOPIs (EOPI-related polypeptide) or EOPI-fusion proteins said method comprising:
(a) in a first aliquot, contacting a candidate agent with the EOPI, EOPI fragment, EOPI-related polypeptide or EOPI-fusion protein, and
(b) determining and comparing the activity of the EOPI, EOPI fragment, EOPI-related polypeptide or EOPI-fusion protein in the first aliquot after addition of the candidate agent with the activity of the EOPI, EOPI fragment, EOPI-related polypeptide or EOPI-fusion protein in a control aliquot, or with a previously determined reference range.

33. The method according to claim 23, wherein the EOPI, EOPI fragment, EOPI-related polypeptide, or EOPI-fusion protein is a recombinant protein.

34. The method according to claim 23, wherein the EOPI, EOPI fragment, EOPI-related polypeptide or EOPI-fusion protein is immobilized on a solid phase.

35. A method for screening or diagnosis of ErbB2 related cancer in a subject or for monitoring the effect of an anti-ErbB2 related cancer drug or therapy administered to a subject, comprising:
(a) contacting at least one oligonucleotide probe comprising 10 or more consecutive nucleotides complementary to a nucleotide sequence encoding an EOPI as defined in claim 3 with RNA obtained from a biological sample from the subject or with cDNA copied from the RNA wherein said contacting occurs under conditions that permit hybridization of the probe to the nucleotide sequence if present;
(b) detecting hybridization, if any, between the probe and the nucleotide sequence; and
(c) comparing the hybridization, if any, detected in step (b) with the hybridization detected in a control sample, or with a previously determined reference range.

36. The method as claimed in claim 35, wherein step (a) includes the step of hybridizing the nucleotide sequence to a DNA array, wherein one or more members of the array are the probes complementary to a plurality of nucleotide sequences encoding distinct EOPIs.

37. A method of modulating the activity of one or more of the ErbB2 Overexpression Protein Isoforms as defined in claim 3, comprising administering to a subject an agent identified by the method of claim 23.

38. A method of treating or preventing ErbB2 related cancer comprising administering to a subject in need of such treatment or prevention a therapeutically effetive dose of an agent that modulates the activity of one or more of the ErbB2 Overexpression Protein Isoforms as defined in claim 3, whereby the symptoms of the ErbB2 related cancer are ameliorated.

39. A method for identifying targets for therapeutic modulation of ErbB2 related cancer wherein the activity of one or more of the ErbB2 Overexpression Protein Isoforms as defined in claim 3, is utilized as a measure to determine whether a candidate target is effective for modulation of ErbB2 related cancer.

40. A method according to claim 3 wherein the ErbB2 related cancer is selected from breast, ovary, stomach or lung cancer.

41. A method according to claim 40 wherein the ErbB2 related cancer is breast cancer.


SEQUENCE LISTING (269 sequences)

SEQ ID NO: 1 - protein, 12 aa, Homo sapiens
AVENSSTAIGIR

SEQ ID NO: 2 - protein, 13 aa, Homo sapiens
DIPNENEAQFQIR

SEQ ID NO: 3 - protein, 16 aa, Homo sapiens
DSYLILETLPTEYDSR

SEQ ID NO: 4 - protein, 12 aa, Homo sapiens
EGVKFDESEKTK

SEQ ID NO: 5 - protein, 13 aa, Homo sapiens
ENVVQSVTSVAEK

SEQ ID NO: 6 - protein, 7 aa, Homo sapiens
ILIEQNR

SEQ ID NO: 7 - protein, 9 aa, Homo sapiens
ISSPTETER

SEQ ID NO: 8 - protein, 14 aa, Homo sapiens
IVGPEENLSQAEAR

SEQ ID NO: 9 - protein, 15 aa, Homo sapiens
LLAVAATAPPDAPNR

SEQ ID NO: 10 - protein, 9 aa, Homo sapiens
QEGDTFYIK

SEQ ID NO: 11 - protein, 16 aa, Homo sapiens
TEFLSFMNTELAAFTK

SEQ ID NO: 12 - protein, 16 aa, Homo sapiens
TVEEAENIAVTSGVVR

SEQ ID NO: 13 - protein, 9 aa, Homo sapiens
VLGVNVMLR

SEQ ID NO: 14 - protein, 11 aa, Homo sapiens
ADQFEYVMYGK

SEQ ID NO: 15 - protein, 11 aa, Homo sapiens
ELTPQVVSAAR

SEQ ID NO: 16 - protein, 13 aa, Homo sapiens
IEGDETSTEAATR

SEQ ID NO: 17 - protein, 11 aa, Homo sapiens
LTHLHEGLPVK

SEQ ID NO: 18 - protein, 12 aa, Homo sapiens
SEDYVDIVQGNR

SEQ ID NO: 19 - protein, 10 aa, Homo sapiens
SNFGYNIPLK

SEQ ID NO: 20 - protein, 9 aa, Homo sapiens
VFQVEYAMK

SEQ ID NO: 21 - protein, 11 aa, Homo sapiens
ADLEMQIENLK

SEQ ID NO: 22 - protein, 11 aa, Homo sapiens
ADQIETQQLMR

SEQ ID NO: 23 - protein, 12 aa, Homo sapiens
AEAGDNLGALVR

SEQ ID NO: 24 - protein, 15 aa, Homo sapiens
AEEYEFLTPVEEAPK

SEQ ID NO: 25 - protein, 9 aa, Homo sapiens
AELSEGQVR

SEQ ID NO: 26 - protein, 11 aa, Homo sapiens
AENYDIPSADR

SEQ ID NO: 27 - protein, 18 aa, Homo sapiens
AFLASPEYVNLPINGNGK

SEQ ID NO: 28 - protein, 10 aa, Homo sapiens
AGFAGDDAPR

SEQ ID NO: 29 - protein, 13 aa, Homo sapiens
AGLENTVAETECR

SEQ ID NO: 30 - protein, 10 aa, Homo sapiens
AGVDPLVPLR

SEQ ID NO: 31 - protein, 8 aa, Homo sapiens
AIHSWLTR

SEQ ID NO: 32 - protein, 15 aa, Homo sapiens
AILVDLEPGTMDSVR

SEQ ID NO: 33 - protein, 12 aa, Homo sapiens
AKQEELEAALQR

SEQ ID NO: 34 - protein, 13 aa, Homo sapiens
ALGQNPTNAEVLK

SEQ ID NO: 35 - protein, 13 aa, Homo sapiens
ALIAAQYSGAQVR

SEQ ID NO: 36 - protein, 10 aa, Homo sapiens
APDFVFYAPR

SEQ ID NO: 37 - protein, 16 aa, Homo sapiens
APVPGTPDSLSSGSSR

SEQ ID NO: 38 - protein, 14 aa, Homo sapiens
AQIHDLVLVGGSTR

SEQ ID NO: 39 - protein, 11 aa, Homo sapiens
AQNLNPMVDVK

SEQ ID NO: 40 - protein, 10 aa, Homo sapiens
AQQELEEQTR

SEQ ID NO: 41 - protein, 8 aa, Homo sapiens
AQYDELAR

SEQ ID NO: 42 - protein, 12 aa, Homo sapiens
ASSVVVSGTPIR

SEQ ID NO: 43 - protein, 13 aa, Homo sapiens
ATDAEADVASLNR

SEQ ID NO: 44 - protein, 11 aa, Homo sapiens
AVAQALEVIPR

SEQ ID NO: 45 - protein, 12 aa, Homo sapiens
AVENSSTAIGIR

SEQ ID NO: 46 - protein, 9 aa, Homo sapiens
AYELALYLR

SEQ ID NO: 47 - protein, 10 aa, Homo sapiens
AYGELPEHAK

SEQ ID NO: 48 - protein, 12 aa, Homo sapiens
DAGVIAGLNVLR

SEQ ID NO: 49 - protein, 13 aa, Homo sapiens
DAINQGMDEELER

SEQ ID NO: 50 - protein, 14 aa, Homo sapiens
DELNADHPFIYIIR

SEQ ID NO: 51 - protein, 10 aa, Homo sapiens
DFLLQQTMLR

SEQ ID NO: 52 - protein, 10 aa, Homo sapiens
DLQNVNITLR

SEQ ID NO: 53 - protein, 8 aa, Homo sapiens
DLTDYLMK

SEQ ID NO: 54 - protein, 7 aa, Homo sapiens
DPLELFR

SEQ ID NO: 55 - protein, 13 aa, Homo sapiens
DQGTYEDYVEGLR

SEQ ID NO: 56 - protein, 10 aa, Homo sapiens
DVLDVYIEHR

SEQ ID NO: 57 - protein, 15 aa, Homo sapiens
DVPFGFQTVTSDVNK

SEQ ID NO: 58 - protein, 15 aa, Homo sapiens
DVQGTDASLDEELDR

SEQ ID NO: 59 - protein, 14 aa, Homo sapiens
DYLLLVMEGTDDGR

SEQ ID NO: 60 - protein, 10 aa, Homo sapiens
EAFNMIDQNR

SEQ ID NO: 61 - protein, 8 aa, Homo sapiens
EAFQLFDR

SEQ ID NO: 62 - protein, 11 aa, Homo sapiens
EALEVDWSSEK

SEQ ID NO: 63 - protein, 11 aa, Homo sapiens
EDGQEYAQVIK

SEQ ID NO: 64 - protein, 10 aa, Homo sapiens
EDQTEYLEER

SEQ ID NO: 65 - protein, 17 aa, Homo sapiens
EEAGGGISEEEAAQYDR

SEQ ID NO: 66 - protein, 16 aa, Homo sapiens
EEFGAEPELAVSAPGR

SEQ ID NO: 67 - protein, 15 aa, Homo sapiens
EEIIGNGEQQYVYLK

SEQ ID NO: 68 - protein, 15 aa, Homo sapiens
EEVVTVETWQEGSLK

SEQ ID NO: 69 - protein, 13 aa, Homo sapiens
EFHLNESGDPSSK

SEQ ID NO: 70 - protein, 13 aa, Homo sapiens
EGFFTNGLTLGAK

SEQ ID NO: 71 - protein, 17 aa, Homo sapiens
EGGGNNLYGEEMVQALK

SEQ ID NO: 72 - protein, 10 aa, Homo sapiens
EHALTSGTIK

SEQ ID NO: 73 - protein, 9 aa, Homo sapiens
EIPSHVLSK

SEQ ID NO: 74 - protein, 13 aa, Homo sapiens
ELAEQLGLSTGEK

SEQ ID NO: 75 - protein, 8 aa, Homo sapiens
ELETVDFK

SEQ ID NO: 76 - protein, 12 aa, Homo sapiens
ELSAVTFPDIIR

SEQ ID NO: 77 - protein, 11 aa, Homo sapiens
EPVDFEQWIEK

SEQ ID NO: 78 - protein, 7 aa, Homo sapiens
EQFVEFR

SEQ ID NO: 79 - protein, 9 aa, Homo sapiens
EQQILTLFR

SEQ ID NO: 80 - protein, 10 aa, Homo sapiens
FADLSEAANR

SEQ ID NO: 81 - protein, 10 aa, Homo sapiens
FDAGELITQR

SEQ ID NO: 82 - protein, 10 aa, Homo sapiens
FEELNADLFR

SEQ ID NO: 83 - protein, 18 aa, Homo sapiens
FGGNPGGFGNQGGFGNSR

SEQ ID NO: 84 - protein, 11 aa, Homo sapiens
FNVWDTAGQEK

SEQ ID NO: 85 - protein, 16 aa, Homo sapiens
FQDGDLTLYQSNTILR

SEQ ID NO: 86 - protein, 9 aa, Homo sapiens
FQTATVTEK

SEQ ID NO: 87 - protein, 16 aa, Homo sapiens
FSLVGIGGQDLNEGNR

SEQ ID NO: 88 - protein, 8 aa, Homo sapiens
FSSLPLGR

SEQ ID NO: 89 - protein, 8 aa, Homo sapiens
FSWFAGEK

SEQ ID NO: 90 - protein, 11 aa, Homo sapiens
FTDEEVDELYR

SEQ ID NO: 91 - protein, 9 aa, Homo sapiens
FTEYETQVK

SEQ ID NO: 92 - protein, 15 aa, Homo sapiens
FVSSGSGGGYGGGMR

SEQ ID NO: 93 - protein, 11 aa, Homo sapiens
GAGTDDHTLIR

SEQ ID NO: 94 - protein, 18 aa, Homo sapiens
GAISAEVYTEEDAASYVR

SEQ ID NO: 95 - protein, 14 aa, Homo sapiens
GASQAGMTGYGMPR

SEQ ID NO: 96 - protein, 17 aa, Homo sapiens
GDEEGVPAVVIDMSGLR

SEQ ID NO: 97 - protein, 17 aa, Homo sapiens
GDTANEIGQVLHFENVK

SEQ ID NO: 98 - protein, 9 aa, Homo sapiens
GIPAPEEER

SEQ ID NO: 99 - protein, 9 aa, Homo sapiens
GLFEVNPWK

SEQ ID NO: 100 - protein, 9 aa, Homo sapiens
GNVGFVFTK

SEQ ID NO: 101 - protein, 12 aa, Homo sapiens
GQLEALQVDGGR

SEQ ID NO: 102 - protein, 10 aa, Homo sapiens
GVALSNVVHK

SEQ ID NO: 103 - protein, 12 aa, Homo sapiens
GVPFTLTTVDTR

SEQ ID NO: 104 - protein, 14 aa, Homo sapiens
GVVDSEDLPLNISR

SEQ ID NO: 105 - protein, 9 aa, Homo sapiens
GYDFPAVLR

SEQ ID NO: 106 - protein, 10 aa, Homo sapiens
GYSFTTTAER

SEQ ID NO: 107 - protein, 11 aa, Homo sapiens
HFSVEGQLEFR

SEQ ID NO: 108 - protein, 17 aa, Homo sapiens
HIFSEDTSDFSGMSETK

SEQ ID NO: 109 - protein, 10 aa, Homo sapiens
HIYYITGETK

SEQ ID NO: 110 - protein, 11 aa, Homo sapiens
HLIEQDFPGMR

SEQ ID NO: 111 - protein, 8 aa, Homo sapiens
HLTGEFEK

SEQ ID NO: 112 - protein, 9 aa, Homo sapiens
HSAESQILK

SEQ ID NO: 113 - protein, 10 aa, Homo sapiens
IDIIPNPQER

SEQ ID NO: 114 - protein, 10 aa, Homo sapiens
IDYIAGLDSR

SEQ ID NO: 115 - protein, 10 aa, Homo sapiens
IEEELGDEAR

SEQ ID NO: 116 - protein, 12 aa, Homo sapiens
IFTSIGEDYDER

SEQ ID NO: 117 - protein, 9 aa, Homo sapiens
IIELPFQNK

SEQ ID NO: 118 - protein, 16 aa, Homo sapiens
IIGATDSSGELMFLMK

SEQ ID NO: 119 - protein, 9 aa, Homo sapiens
IINTPEVVR

SEQ ID NO: 120 - protein, 10 aa, Homo sapiens
IIQLLDDYPK

SEQ ID NO: 121 - protein, 9 aa, Homo sapiens
IIVLGLLPR

SEQ ID NO: 122 - protein, 11 aa, Homo sapiens
ILDEIEEHNIK

SEQ ID NO: 123 - protein, 13 aa, Homo sapiens
ILDSVGIEADDDR

SEQ ID NO: 124 - protein, 7 aa, Homo sapiens
ILIEELR

SEQ ID NO: 125 - protein, 7 aa, Homo sapiens
ILIEQNR

SEQ ID NO: 126 - protein, 10 aa, Homo sapiens
ILVATNLFGR

SEQ ID NO: 127 - protein, 10 aa, Homo sapiens
ILYLTPEQEK

SEQ ID NO: 128 - protein, 16 aa, Homo sapiens
IMEGPAFNFLDAPAVR

SEQ ID NO: 129 - protein, 16 aa, Homo sapiens
INPDGSQSVVEVPYAR

SEQ ID NO: 130 - protein, 12 aa, Homo sapiens
IPQSTLSEFYPR

SEQ ID NO: 131 - protein, 10 aa, Homo sapiens
IQLVEEELDR

SEQ ID NO: 132 - protein, 10 aa, Homo sapiens
ISEQFTAMFR

SEQ ID NO: 133 - protein, 9 aa, Homo sapiens
ITMQNLNDR

SEQ ID NO: 134 - protein, 12 aa, Homo sapiens
ITSGPFEPDLYK

SEQ ID NO: 135 - protein, 14 aa, Homo sapiens
IVIGMDVAASEFYR

SEQ ID NO: 136 - protein, 9 aa, Homo sapiens
IVVVTAGVR

SEQ ID NO: 137 - protein, 12 aa, Homo sapiens
KPLLESGTLGTK

SEQ ID NO: 138 - protein, 10 aa, Homo sapiens
KVIDDTNITR

SEQ ID NO: 139 - protein, 15 aa, Homo sapiens
LAGTQPLEVLEAVQR

SEQ ID NO: 140 - protein, 10 aa, Homo sapiens
LAVNMVPFPR

SEQ ID NO: 141 - protein, 10 aa, Homo sapiens
LDGEASINNR

SEQ ID NO: 142 - protein, 16 aa, Homo sapiens
LEAELGNMQGLVEDFK

SEQ ID NO: 143 - protein, 9 aa, Homo sapiens
LEAPLEELR

SEQ ID NO: 144 - protein, 13 aa, Homo sapiens
LEEGPPVTTVLTR

SEQ ID NO: 145 - protein, 12 aa, Homo sapiens
LEGLTDEINFLR

SEQ ID NO: 146 - protein, 11 aa, Homo sapiens
LENGEIETIAR

SEQ ID NO: 147 - protein, 16 aa, Homo sapiens
LEQPDPGAVAAAAILR

SEQ ID NO: 148 - protein, 12 aa, Homo sapiens
LEQYTSAIEGTK

SEQ ID NO: 149 - protein, 13 aa, Homo sapiens
LEVEANNAFDQYR

SEQ ID NO: 150 - protein, 10 aa, Homo sapiens
LFADAEEEQR

SEQ ID NO: 151 - protein, 11 aa, Homo sapiens
LFELEEQDLFR

SEQ ID NO: 152 - protein, 10 aa, Homo sapiens
LGFEDGSVLK

SEQ ID NO: 153 - protein, 10 aa, Homo sapiens
LGIHEDSQNR

SEQ ID NO: 154 - protein, 16 aa, Homo sapiens
LIAPVAEEEATVPNNK

SEQ ID NO: 155 - protein, 10 aa, Homo sapiens
LLDYVPIGPR

SEQ ID NO: 156 - protein, 11 aa, Homo sapiens
LLHQLADLVER

SEQ ID NO: 157 - protein, 7 aa, Homo sapiens
LLLCNPR

SEQ ID NO: 158 - protein, 13 aa, Homo sapiens
LLLEFTDTSYEEK

SEQ ID NO: 159 - protein, 10 aa, Homo sapiens
LLLNNDNLLR

SEQ ID NO: 160 - protein, 9 aa, Homo sapiens
LLPDDPYEK

SEQ ID NO: 161 - protein, 9 aa, Homo sapiens
LLPLEEHYR

SEQ ID NO: 162 - protein, 7 aa, Homo sapiens
LMEDLDR

SEQ ID NO: 163 - protein, 16 aa, Homo sapiens
LNLEAINYMAADGDFK

SEQ ID NO: 164 - protein, 9 aa, Homo sapiens
LNYKPPPQK

SEQ ID NO: 165 - protein, 8 aa, Homo sapiens
LPDGYEFK

SEQ ID NO: 166 - protein, 14 aa, Homo sapiens
LPGELEPVQATQNK

SEQ ID NO: 167 - protein, 8 aa, Homo sapiens
LQDAEIAR

SEQ ID NO: 168 - protein, 8 aa, Homo sapiens
LQEEMLQR

SEQ ID NO: 169 - protein, 8 aa, Homo sapiens
LRDDTELR

SEQ ID NO: 170 - protein, 10 aa, Homo sapiens
LSELEAALQR

SEQ ID NO: 171 - protein, 11 aa, Homo sapiens
LSNLALVKPEK

SEQ ID NO: 172 - protein, 9 aa, Homo sapiens
LSPEELLLR

SEQ ID NO: 173 - protein, 13 aa, Homo sapiens
LSVLGAITSVQQR

SEQ ID NO: 174 - protein, 10 aa, Homo sapiens
LVIIESDLER

SEQ ID NO: 175 - protein, 10 aa, Homo sapiens
MFGGPGTASR

SEQ ID NO: 176 - protein, 14 aa, Homo sapiens
MIGFPSSAGSVSPR

SEQ ID NO: 177 - protein, 8 aa, Homo sapiens
MLEQLDMR

SEQ ID NO: 178 - protein, 11 aa, Homo sapiens
MLLADQGQSWK

SEQ ID NO: 179 - protein, 14 aa, Homo sapiens
MTDQEAIQDLWQWR

SEQ ID NO: 180 - protein, 11 aa, Homo sapiens
MVVESAYEVIK

SEQ ID NO: 181 - protein, 18 aa, Homo sapiens
NEEDAAELVALAQAVNAR

SEQ ID NO: 182 - protein, 11 aa, Homo sapiens
NFIAQGPYENR

SEQ ID NO: 183 - protein, 12 aa, Homo sapiens
NFSGAELEGLVR

SEQ ID NO: 184 - protein, 13 aa, Homo sapiens
NGESSELDLQGIR

SEQ ID NO: 185 - protein, 10 aa, Homo sapiens
NHEEEMNALR

SEQ ID NO: 186 - protein, 8 aa, Homo sapiens
NLLSVAYK

SEQ ID NO: 187 - protein, 16 aa, Homo sapiens
NPVPAQDEALYQQLLR

SEQ ID NO: 188 - protein, 13 aa, Homo sapiens
NSILAQVLDQSAR

SEQ ID NO: 189 - protein, 11 aa, Homo sapiens
NVIGLQMGTNR

SEQ ID NO: 190 - protein, 7 aa, Homo sapiens
NVPNWHR

SEQ ID NO: 191 - protein, 11 aa, Homo sapiens
PPYTVVYFPVR

SEQ ID NO: 192 - protein, 9 aa, Homo sapiens
QAPIPAELR

SEQ ID NO: 193 - protein, 12 aa, Homo sapiens
QAQEYEALLNIK

SEQ ID NO: 194 - protein, 13 aa, Homo sapiens
QEYDESGPSIVHR

SEQ ID NO: 195 - protein, 8 aa, Homo sapiens
QFLSETEK

SEQ ID NO: 196 - protein, 10 aa, Homo sapiens
QFVTATDVVR

SEQ ID NO: 197 - protein, 14 aa, Homo sapiens
QMEQISQFLQAAER

SEQ ID NO: 198 - protein, 12 aa, Homo sapiens
QPQVAELLAEAR

SEQ ID NO: 199 - protein, 10 aa, Homo sapiens
QPVLSQTEAR

SEQ ID NO: 200 - protein, 8 aa, Homo sapiens
QQLEELAR

SEQ ID NO: 201 - protein, 11 aa, Homo sapiens
QSVENDIHGLR

SEQ ID NO: 202 - protein, 8 aa, Homo sapiens
QTFGYGAR

SEQ ID NO: 203 - protein, 8 aa, Homo sapiens
QVLEPSFR

SEQ ID NO: 204 - protein, 8 aa, Homo sapiens
RFEELGVK

SEQ ID NO: 205 - protein, 9 aa, Homo sapiens
RLPPQQIEK

SEQ ID NO: 206 - protein, 10 aa, Homo sapiens
RNEFLGELQK

SEQ ID NO: 207 - protein, 10 aa, Homo sapiens
SADTLWDIQK

SEQ ID NO: 208 - protein, 12 aa, Homo sapiens
SAYGGPVGAGIR

SEQ ID NO: 209 - protein, 14 aa, Homo sapiens
SDIDEIVLVGGSTR

SEQ ID NO: 210 - protein, 11 aa, Homo sapiens
SENEEFVEVGR

SEQ ID NO: 211 - protein, 13 aa, Homo sapiens
SFPDFPTPGVVFR

SEQ ID NO: 212 - protein, 7 aa, Homo sapiens
SLDFYTR

SEQ ID NO: 213 - protein, 9 aa, Homo sapiens
SLFNYHDTR

SEQ ID NO: 214 - protein, 16 aa, Homo sapiens
SLGLSLSGGDQEDAGR

SEQ ID NO: 215 - protein, 13 aa, Homo sapiens
SLGSVQAPSYGAR

SEQ ID NO: 216 - protein, 14 aa, Homo sapiens
SLYASSPGGVYATR

SEQ ID NO: 217 - protein, 10 aa, Homo sapiens
SMQDVVEDFK

SEQ ID NO: 218 - protein, 10 aa, Homo sapiens
SNFGYNIPLK

SEQ ID NO: 219 - protein, 15 aa, Homo sapiens
SNMDNMFESYINNLR

SEQ ID NO: 220 - protein, 12 aa, Homo sapiens
SPQNQYPAELMR

SEQ ID NO: 221 - protein, 14 aa, Homo sapiens
SQGGEPTYNVAVGR

SEQ ID NO: 222 - protein, 9 aa, Homo sapiens
SQLLSYIDR

SEQ ID NO: 223 - protein, 10 aa, Homo sapiens
SSYYMIGEQK

SEQ ID NO: 224 - protein, 8 aa, Homo sapiens
STFSTNYR

SEQ ID NO: 225 - protein, 16 aa, Homo sapiens
SYELPDGQVITIGNER

SEQ ID NO: 226 - protein, 10 aa, Homo sapiens
SYELQESNVR

SEQ ID NO: 227 - protein, 11 aa, Homo sapiens
TDYMVGSYGPR

SEQ ID NO: 228 - protein, 11 aa, Homo sapiens
TEEGPTLSYGR

SEQ ID NO: 229 - protein, 8 aa, Homo sapiens
TEISEMNR

SEQ ID NO: 230 - protein, 13 aa, Homo sapiens
TELISVSEVHPSR

SEQ ID NO: 231 - protein, 11 aa, Homo sapiens
TFDQLTPEESK

SEQ ID NO: 232 - protein, 11 aa, Homo sapiens
TIIQNPTDQQK

SEQ ID NO: 233 - protein, 12 aa, Homo sapiens
TIISYIDEQFER

SEQ ID NO: 234 - protein, 13 aa, Homo sapiens
TLLGDGPVVTDPK

SEQ ID NO: 235 - protein, 15 aa, Homo sapiens
TQNPMVTGTSVLGVK

SEQ ID NO: 236 - protein, 12 aa, Homo sapiens
TSFFQALGITTK

SEQ ID NO: 237 - protein, 13 aa, Homo sapiens
TTPSYVAFTDTER

SEQ ID NO: 238 - protein, 16 aa, Homo sapiens
TVLSGGTTMYPGIADR

SEQ ID NO: 239 - protein, 14 aa, Homo sapiens
TVQIEASTVEIEER

SEQ ID NO: 240 - protein, 13 aa, Homo sapiens
TVQSLEIDLDSMR

SEQ ID NO: 241 - protein, 12 aa, Homo sapiens
TYDSYLGDDYVR

SEQ ID NO: 242 - protein, 9 aa, Homo sapiens
TYSLGSALR

SEQ ID NO: 243 - protein, 17 aa, Homo sapiens
VAGALAEAGVGLEEIAK

SEQ ID NO: 244 - protein, 12 aa, Homo sapiens
VDALNDEINFLR

SEQ ID NO: 245 - protein, 12 aa, Homo sapiens
VDIIENQVMDFR

SEQ ID NO: 246 - protein, 13 aa, Homo sapiens
VDNDENEHQLSLR

SEQ ID NO: 247 - protein, 13 aa, Homo sapiens
VEEAEPEEFVVEK

SEQ ID NO: 248 - protein, 11 aa, Homo sapiens
VEIIANDQGNR

SEQ ID NO: 249 - protein, 9 aa, Homo sapiens
VELQELNDR

SEQ ID NO: 250 - protein, 10 aa, Homo sapiens
VEVTEFEDIK

SEQ ID NO: 251 - protein, 13 aa, Homo sapiens
VFVEEQVYSEFVR

SEQ ID NO: 252 - protein, 17 aa, Homo sapiens
VLALSVETDYTFPLAEK

SEQ ID NO: 253 - protein, 9 aa, Homo sapiens
VLGMTLIQK

SEQ ID NO: 254 - protein, 9 aa, Homo sapiens
VLTEIIASR

SEQ ID NO: 255 - protein, 10 aa, Homo sapiens
VLTLSDDLER

SEQ ID NO: 256 - protein, 9 aa, Homo sapiens
VNIKPQVDR

SEQ ID NO: 257 - protein, 7 aa, Homo sapiens
VNMEDLR

SEQ ID NO: 258 - protein, 17 aa, Homo sapiens
VTHAVVTVPAYFNDAQR

SEQ ID NO: 259 - protein, 14 aa, Homo sapiens
YDPPLEDGAMPSAR

SEQ ID NO: 260 - protein, 11 aa, Homo sapiens
YEEIDNAPEER

SEQ ID NO: 261 - protein, 7 aa, Homo sapiens
YIQHTYR

SEQ ID NO: 262 - protein, 11 aa, Homo sapiens
YLAEFATGNDR

SEQ ID NO: 263 - protein, 9 aa, Homo sapiens
YLTVATVFR

SEQ ID NO: 264 - protein, 15 aa, Homo sapiens
YMTISGFQIEETIDR

SEQ ID NO: 265 - protein, 9 aa, Homo sapiens
YPQLLPGIR

SEQ ID NO: 266 - protein, 14 aa, Homo sapiens
YSLVLELSDSGAFR

SEQ ID NO: 267 - protein, 10 aa, Homo sapiens
YTYEHDPITK

SEQ ID NO: 268 - protein, 7 aa, Homo sapiens
YVQHTYR

SEQ ID NO: 269 - protein, 15 aa, Homo sapiens
ILDQGEDFPASEMTR